(19) United States
(12) Patent Application Publication
(10) Pub. No.: US 2018/0372720 A1
(43) Pub. Date: Dec. 27, 2018

(54) STABLE ISOTOPE LABELING KINETICS - SECONDARY ION MASS SPECTROMETRY (SILK SIMS) AND METHODS OF USE THEREOF

(71) Applicant: Washington University, St. Louis, MO (US)

(72) Inventors: Norelle C. Wildburger, St. Louis, MO (US); Randall J. Bateman, St. Louis, MO (US); Donald Elbert, Auston, TX (US); Frank Gyngard, St. Louis, MO (US)

(21) Appl. No.: 16/017,623

(22) Filed: Jun. 25, 2018

Related U.S. Application Data
(60) Provisional application No. 62/523,811, filed on Jun. 23, 2017.

(51) Int. Cl. G01N 33/50

(52) U.S. Cl. CPC G01N 33/502; G01N 2800/52; G01N 33/5088

(57) ABSTRACT

The present disclosure provides for methods or systems for measuring a biomolecule or a therapeutic agent metabolism and determining the biomolecule or therapeutic agent location in a biological sample. Stable Isotope Labeling Kinetics-Secondary Ion Mass Spectrometry (SILK-SIMS) can be utilized for the simultaneous detection, quantification, and imaging of biomolecules or therapeutic agents.

### CROSS-REFERENCE TO RELATED APPLICATION

This application claims the benefit of U.S. Provisional Application No. 62/523,811, filed Jun. 23, 2017, the disclosures of which is hereby incorporated by reference in its entirety.

### FIELD OF THE INVENTION

The present disclosure generally relates to methods and systems for detecting biomolecules or therapeutic agents, including in situ spatial imaging, mapping, and detection of dynamic metabolic processes. Additionally, the methods and systems can be used to make diagnostic and theragonstic determinations in subjects.

### BACKGROUND

Cancer development involves dynamic and reciprocal interactions between neoplastic cells, activated stromal cells, extracellular matrix (ECM) and soluble molecules in their vicinity. Together these environmental factors foster the malignant phenotype. Intertwined with these hallmarks of cancer development is the fact that tumor cells metabolize glucose largely via aerobic glycolysis as opposed to oxidative phosphorylation, and produce lactate in a less energy-efficient manner, i.e., the Warburg effect. This distinct metabolic state is common to most solid tumors, including breast cancers, and is thought to contribute to their chemo-resistance. Thus altered metabolism may limit efficacy of standard anti-cancer therapy, but this feature may also be used to identify and characterize subtypes of neoplastic tissue.

Pathological techniques have always been critical in the diagnosis and treatment of cancer. Classic morphologic criteria, based on vital dyes and light microscopy, have been complemented by immunohistochemistry and gene expression profiling, leading to histological markers of growth factor receptor status or transcriptomic signatures that, for example, predict an individual's likely treatment response. However, all current histological analyses are ‘blind’ to the spatially ordered metabolic dynamics of the tumor. Metabolic fluxes are closer to function than static markers and may therefore correlate better with phenotypic behavior.

Alzheimer's Disease (AD) is the most common cause of dementia and is an increasing public health problem. AD, like other central nervous system (CNS) degenerative diseases, is characterized by disturbances in protein production, accumulation, and clearance. In AD, dysregulation in the metabolism of the protein, amyloid-beta (Aβ), is indicated by a massive buildup of this protein in the brains of those with the disease. Because of the severity and increasing prevalence of this disease in the population, it is urgent that better treatments be developed.

A need exists, therefore, for methods and systems for analyzing the in vivo kinetics of biomolecules or therapeutic agents in a variety of pathologies. In particular, methods and systems are needed for modeling metabolic flux, kinetic measurement, and the localization of biomolecules associated with disease state, or progression and therapeutic agents relating to efficacy and resistance. Such a model or system may serve as a useful tool in the characterization and treatment of the underlying processes of disease.

### SUMMARY

Among the various aspects of the present disclosure is the provision of using SILK-SIMS for measuring biomolecule or therapeutic agent metabolism and determining the biomolecule or therapeutic agent location in a biological sample.

One aspect of the present disclosure is directed to a method for measuring biomolecule or therapeutic agent metabolism and determining the biomolecule or therapeutic agent in a biological sample.

Another aspect of the present disclosure is directed to a method of a system for measuring biomolecule or therapeutic agent metabolism and determining the biomolecule or therapeutic agent location in a biological sample.

In another aspect, the present disclosure provides imaging a biological sample using Stable Isotope Labeling Kinetics (SILK) and nanoscale secondary ion mass spectrometry (NanoSIMS); spatially detecting a deposition of a protein into amyloid plaques in AD brain; quantifying a deposition of a protein into amyloid plaques in AD brain; or localizing and quantifying the stable, non-radioactive isotope ¹³C in a biological sample.

In still another aspect, the present disclosure provides a method or system comprising (i) electrostatically rastering a focused Cs+ primary ion beam across a defined region-of-interest (ROI) in a biological sample (e.g., tissue) producing secondary ions used to measure the atomic composition of the biological sample surface; (ii) producing high-resolution quantitative image representing an in situ isotopic-histological map at 100 nm lateral resolution; (iii) acquiring one or more isotopes, optionally, in parallel; (iv) detecting and localizing a stable, non-radioactive isotope tracer; or (v) quantitatively imaging the ratio of two stable isotopes of the same element.

Other aspects and iterations of the disclosure are described in more detail below.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A and FIG. 1B depict graphically the linearity of response in NanoSIMS measurements of cells treated with increasing percent of ¹³C₆-leucine (R²=0.99546) and a raw and normalized y-intercept of 0.0106±1.53×10⁻⁴ and 0.0111±2.25×10⁻⁴, respectively.

FIG. 2A, FIG. 2B, FIG. 2C, FIG. 2D, FIG. 2E, FIG. 2F, FIG. 2G, FIG. 2H and FIG. 2I show the feasibility of detecting ¹³C₆-leucine isotopic enrichment in native amyloid-β plaques. This figure shows the administered tracer to two APP/PS1 mice at 3.5 months of age (pre-plaque pathology) for 10 and 5 weeks (FIG. 2A). The APP/PS1 mouse labeled for 10 consecutive weeks (FIG. 2B-FIG. 2I) reached raw and normalized ¹³C¹⁴N—/¹²C¹⁴N— ratios of 0.023±1.41×10⁻⁵ and 0.024±3.43×10⁻⁴ respectively, compared to background (i.e., brain parenchyma) in Area 1 (0.021±1.28×10⁻⁵ and 0.022±3.15×10⁻⁴, raw and normalized) and Area 2 (0.021±1.13×10⁻⁵ and 0.022±3.19×10⁻⁴, raw and normalized) (FIG. 8).

FIG. 3A and FIG. 3B shows an illustration infusion of ¹³C₆-leucine for 9 hours in hospice care patients. After time of death (range of days to months), the brain is donated through autopsy and processed for NanoSIMS imaging. Data will be used to computationally model Aβ kinetics in vivo based on isotopic label.

FIG. 4A, FIG. 4B, FIG. 4C, FIG. 4D, FIG. 4E, FIG. 4F, FIG. 4G and FIG. 4H show the image quality of carbon imaged as monoisotopes (¹²C— and ¹³C—) and polyatomic isotopes (¹²C¹⁴N— and ¹³C¹⁴N—). FIG. 4A-FIG. 4D 50×50 μm image of 0% ¹³C₆-leucine labeled cells imaged by NanoSIMS with four electron multipliers set to detect ¹²C—, ¹³C—, ¹²C¹⁴N—, and ¹³C¹⁴N— ions, respectively. FIG. 4AE-FIG. 4H 45×45 μm image of an unlabeled human AD plaque imaged by NanoSIMS as ¹²C—, ¹³C—, ¹²C¹⁴N—, and ¹³C¹⁴N— ions, respectively. Apparent from panels FIG. 4B, FIG. 4F and FIG. 4D, FIG. 4H is the improved image quality and morphology, and Cts/s when carbon is imaged as a polyatomic isotope (i.e., cyanide ion) compared to carbon monoisotopes, panels FIG. 4A, FIG. 4E and FIG. 4C, FIG. 4G. Scale bar, 5 μm.

FIG. 5A, FIG. 5B, FIG. 5C, FIG. 5D, FIG. 5E and FIG. 5F show nitrogen contribution to improved image quality when imaging using CN⁻. FIG. 5A, FIG. 5D ¹²C— ion map. FIG. 5B, FIG. 5E ¹²C¹⁴N— ion map. FIG. 5C, FIG. 5F The ratio of ¹²C¹⁴N/¹²C to deduce the nitrogen contribution of the polyatomic isotope image in 0% ¹³C₆-leucine labeled cells (50×50 μm) and unlabeled human AD plaque (45×45 μm), respectively. Scale bar, 5 μm.

FIG. 6 shows quantitative improvement in image analysis using polyatomic carbon isotopes versus monoisotopes. Raw and normalized values of ¹³C/¹²C and ¹³C¹⁴N/¹²C¹⁴N ratios for 0% ¹³C₆-leucine labeled cells (50×50 μm), unlabeled human AD plaque (PtA40; 45×45 μm), and SILK participant (Pt5; 100×100 μm) with a delta of 4.5 yrs between labeling and expiration. Raw values represent the mean±S.D. (Poisson errors) of all ¹³C/¹²C and ¹³C¹⁴N/¹²C¹⁴N ratios across the entire image over all cycles of that image. Normalized values are the raw ratios normalized to the theoretical natural abundance of ¹³C (0.011123471) and their respective standard deviations were calculated as the sum in quadrature of the standard deviation of the average ratios measured for non-labeled material and the Poisson errors of the feature itself. Dashed horizontal line represents natural abundance of ¹³C.

FIG. 7A, FIG. 7B, FIG. 7C, FIG. 7D, FIG. 7E, FIG. 7F, FIG. 7G, and FIG. 7H show quantitative imaging of increasing ¹³C enrichment in cell-based standard curve. ¹²C¹⁴N— and ¹³C¹⁴N— ion maps of 50×50 μm images of B-cell hybridoma given increasing percentages of 13C6-leucine to generate the standard curve seen in FIG. 1. δ¹³C¹⁴N/¹²C¹⁴N images demonstrate on a visual scale the deviation of the raw measurements away from natural abundance (0.011123471) in permil (i.e., parts per thousand, %) pixel-by-pixel. Scale bar, 5 μm.

FIG. 8A and FIG. 8B show ROIs used for isotope enrichment quantitation in APP/PS1 mice. Outline (white) of the ROIs used define senile plaques (S.P.) and areas (#1-2 and #1-4) in APP/PS1 mice labeled for 10 weeks and 5 weeks, respectively for quantitation in FIG. 2.

FIG. 9A, FIG. 9B, FIG. 9C, FIG. 9D, FIG. 9E, FIG. 9F, FIG. 9G and FIG. 9H show ¹³C enrichment in neuron from APP/PS1 mouse labeled for 10 weeks. FIG. 9A Optical image of neuron stained with Toluidine Blue (T. Blue) 60× objective. Scale bar, 10 μm. FIG. 9B Scanning electron microscope image of the same neuron (2,177×). Scale bar, 20 μm. FIG. 9C ¹²C¹⁴N— ion map. FIG. 9D ¹³C¹⁴N— ion map. FIG. 9E ³¹P— ion map. FIG. 9F ³²S— ion map. FIG. 9G δ¹³C¹⁴N/¹²C¹⁴N image demonstrating permil (i.e., parts per thousand, ‰) deviation away of the raw measurements from natural abundance (0.011123471). ROIs used for quantitation of areas 1 and 2 are outline in white and the neuron ROI is outlined in white in FIG. 9C-FIG. 9F. All NanoSIMS images (FIG. 9C-FIG. 9G) are 60×60 μm. Scale bar, 5 μm. FIG. 9H Raw and normalized values of ¹³C¹⁴N/¹²C¹⁴N ratios for ROIs. Normalized values are the raw ratios normalized to the theoretical natural abundance of ¹³C (0.011123471) and 0% labeled cells. Standard deviations were calculated as the sum in quadrature of the standard deviation of the average ratios measured for non-labeled material and the Poisson errors of the feature itself. Dashed horizontal line represents natural abundance of ¹³C. The percent difference between each ROI is shown above the mean±S.D. of raw and normalized ratios.

FIG. 10A, FIG. 10B, FIG. 10C, FIG. 10D, FIG. 10E, and FIG. 10F show the test-retest reliability of ¹³C¹⁴N—/¹²C¹⁴N— ratios in SILK-SIMS analysis of APP/PS1 neuronal and plaque features. FIG. 10A δ¹³C¹⁴N/¹²C¹⁴N of the sum of all the even cycles of the neuronal image (60×60 μm). FIG. 10B δ¹³C¹⁴N/¹²C¹⁴N of the sum of all the odd cycles of the neuronal image. FIG. 10C Test-retest reliability of raw and normalized ¹³C¹⁴N/¹²C¹⁴N ratios in odd versus even only summed cycles for the neuron. FIG. 10D δ¹³C¹⁴N/¹²C¹⁴N image of the sum of all the even cycles of a plaque from a 5-week labeled APP/PS1 mouse (17×17 μm). FIG. 10E δ¹³C¹⁴N/¹²C¹⁴N image of the sum of all the odd cycles of a plaque from a 5-week labeled APP/PS1 mouse. FIG. 10F Test-retest reliability of raw and normalized ¹³C¹⁴N/¹²C¹⁴N ratios in odd versus even only summed cycles for the plaque. Scale bar, 5 μm.

FIG. 11A, FIG. 11B, FIG. 11C, FIG. 11D, FIG. 11E, FIG. 11F, FIG. 11G and FIG. 11H show the test-retest reliability of SILK-SIMS to study APP/PS1 plaque and neuronal features. FIG. 11A-FIG. 11B The δ13C14N/12C14N of the sum of all the even and odd cycles of the neuronal image (60×60 μm), respectively divided into 10×10 pixels ROIs. Scale bar, 5 μm. FIG. 11C Scatter plot illustrates the test-retest reliability coefficient of SILK-SIMS measurements of the neuronal image (Spearman's r=0.9715, R2=0.98269, p<0.0001) plotting each even versus odd cycle 10×10 pixel ROI values against each other. FIG. 11D Likewise, Bland-Altman plot of the average and difference of odd and even cycle sums demonstrate little bias and most values falling within the 95% confidence interval. FIG. 11E-FIG. 11F The δ¹³C¹⁴N/¹²C¹⁴N of the sum of all the even and odd cycles of a plaque from the 5-week labeled APP/PS1 mouse (17×17 μm), respectively divided into 10×10 pixels ROIs. Scale bar, 5 μm. FIG. 11C Scatter plot illustrates the test-retest reliability coefficient of SILK-SIMS measurements of the plaque image (Spearman's r=0.7573, R2=0.75272, p<0.0001) plotting each even versus odd cycle 10×10 pixel ROI values against each other. FIG. 11D Likewise, Bland-Altman plot of the average and difference of odd and even cycle sums demonstrate little bias and most values falling within the 95% confidence interval.

FIG. 12A, FIG. 12B, FIG. 12C, FIG. 12D, FIG. 12E, FIG. 12F, FIG. 12G, FIG. 12H, FIG. 12I and FIG. 12J show the test-retest reliability of NanoSIMS imaging of human AD plaque. FIG. 12A-FIG. 12B The δ¹³C¹⁴N/¹²C¹⁴N of the sum of all the even and odd cycles of the plaque image (80×80 μm), respectively divided into 10×10 pixels ROIs over the plaque feature. Scale bar, 5 μm. FIG. 12C Scatter plot illustrates the test-retest reliability coefficient of SILK-SIMS measurements of the neuronal image (Spearman's r=1, R2=1, p<0.0001) plotting each even versus odd cycle 10×10 pixel ROI values against each other. FIG. 12D Likewise, Bland-Altman plot of the average and difference of odd and even cycle sums demonstrate little bias with all values falling within the 95% confidence interval. FIG. 12E Test-retest reliability of raw and normalized ¹³C¹⁴N/¹²C¹⁴N ratios in odd versus even summed cycles for the plaque. Dashed horizontal line represents natural abundance of ¹³C. FIG. 12F-FIG. 12G The (¹³C¹⁴N/¹²C¹⁴N of the sum of the first 20 and last 20 cycles of the plaque image, respectively divided into 10×10 pixels ROIs over the plaque feature. Scale bar, 5 μm. FIG. 12H Scatter plot illustrates the test-retest reliability coefficient of SILK-SIMS measurements of the plaque image over time—total acquisition time was 11.8 hrs—(Spearman's r=0.9989, R2=0.99784, p<0.0001) plotting the first and last 20 cycles 10×10 pixel ROI values against each other. FIG. 12I Bland-Altman plot of the average and difference of the first and second half cycle sums demonstrate little bias with all values falling within the 95% confidence interval. FIG. 12J Test-retest reliability of raw and normalized ¹³C¹⁴N/¹²C¹⁴N ratios in odd versus even summed cycles for the plaque.

FIG. 13A, FIG. 13B, FIG. 13C, FIG. 13D, FIG. 13E and FIG. 13F show quantitative NanoSIMS imaging of SILK participant with a 4.5 yr delta between labeling and expiration. FIG. 13A Optical image of a plaque (S.P., red) from Pt5 stained with Toluidine Blue (T. Blue) at 40× objective. Scale bar, 40 FIG. 13B Scanning electron microscope image of the same plaque (1,525×). Scale bar, 30 μm. FIG. 13C ¹²C¹⁴N— ion map. FIG. 13D ¹³C¹⁴N— ion map. FIG. 13E (¹³C¹⁴N/¹²C¹⁴N image demonstrating permil (i.e., parts per thousand, ‰) deviation of the raw measurements away from natural abundance (0.011123471). ROIs used for quantitation of areas 1 and 2 are outline in white and the plaque ROI is outlined in white in FIG. 13C-FIG. 13D. All NanoSIMS images (FIG. 13C-FIG. 13E) are 55×55 μm. Scale bar, 5 μm. FIG. 13F Raw and normalized values of ¹³C¹⁴N/¹²C¹⁴N ratios for ROIs. Dashed horizontal line represents natural abundance of ¹³C.

FIG. 14A, FIG. 14B, FIG. 14C, FIG. 14D, FIG. 14E, and FIG. 14F shows quantitative NanoSIMS imaging of an unlabeled AD participant. FIG. 14A Optical image of a plaque (S.P., red) from Pt40 stained with Toluidine Blue (T. Blue) at 40× objective. FIG. 14B Scanning electron microscope image of the same plaque (2,500×). Scale bar, 30 μm. FIG. 14C ¹²C¹⁴N— ion map. FIG. 14D ¹³C¹⁴N— ion map. FIG. 14E δ¹³C¹⁴N/¹²C¹⁴N image demonstrating permil (i.e., parts per thousand, ‰) deviation of the raw measurements away from natural abundance (0.011123471). ROIs used for quantitation of areas 1 and 2 are outline in white and the plaque ROI is outlined in white in FIG. 14C-FIG. 14D. All NanoSIMS images (FIG. 14C-FIG. 14E) are 45×45 μm. Scale bar, 5 μm. FIG. 14F Raw and normalized values of ¹³C¹⁴N/¹²C¹⁴N ratios for ROIs. Dashed horizontal line represents natural abundance of ¹³C.

FIG. 15A, FIG. 15B, FIG. 15C, FIG. 15D, FIG. 15E, FIG. 15F, FIG. 15G, FIG. 15H, FIG. 15I, FIG. 15J, FIG. 15K, and FIG. 15L show quantitative NanoSIMS imaging of a SILK Pt2 plaques in the precuneus. FIG. 15A Optical image of a plaque (S.P., red) from Pt2 stained with Toluidine Blue (T. Blue) at 40× objective. FIG. 15B Scanning electron microscope image of the same plaque (1,248×). Scale bar, 40 μm. FIG. 15C The NanoSIMS image was subdivided into 625 ROIs each representing 10×10 pixels from which carbon ratios were calculated. The histogram plots all ROIs and their respect ¹³C¹⁴N/¹²C¹⁴N ratios. Blue line is the theoretical natural abundance of ¹³C, red line the measured ¹³C¹⁴N/¹²C¹⁴N ratio of an unlabeled AD brain with ±standard deviation in orange, and the mean of the unlabeled sample+2σ in green. FIG. 15D ¹²C¹⁴N— ion map. FIG. 15E ¹³C¹⁴N— ion map. FIG. 15F ¹³C¹⁴N/¹²C¹⁴N image showing the distribution of ¹³C in the sample per pixel; each pixel is the width of the Cs+ beam—100 nm. ROIs that were significantly enriched in ¹³C as described in the Methods and Materials are outline in white and red. Note: NanoSIMS image in FIG. 15D and FIG. 15F are distorted due to a shortage in a lens in the primary column of the instrument. FIG. 15G Optical image of a plaque (S.P., red) from Pt2 stained with Toluidine Blue (T. Blue) at 40× objective. FIG. 15H Scanning electron microscope image of the same plaque (1,250×). Scale bar, 40 μm. FIG. 15I The NanoSIMS image was subdivided into 625 ROIs each representing 10×10 pixels from which carbon ratios were calculated. The histogram plots all ROIs and their respect ¹³C¹⁴N/¹²C¹⁴N ratios. Blue line is the theoretical natural abundance of ¹³C, red line the measured ¹³C¹⁴N/¹²C¹⁴N ratio of an unlabeled AD brain with ±standard deviation in orange, and the mean of the unlabeled sample+2σ in green. FIG. 15J ¹²C¹⁴N— ion map. FIG. 15K ¹³C¹⁴N— ion map. FIG. 15L ¹³C¹⁴N/¹²C¹⁴N image showing the distribution of ¹³C in the sample per pixel; each pixel is the width of the Cs+ beam—100 nm. ROIs that were significantly enriched in ¹³C as described in the Methods and Materials are outline in white and red. All NanoSIMS images (FIG. 15D-FIG. 15F and FIG. 15J-FIG. 15L) are 25×45 μm. Scale bar, 2 μm. All ROIs represent 10×10 pixels (0.98 μm2) or 1.1 μm in diameter.

FIG. 16A, FIG. 16B, FIG. 16C, FIG. 16D, FIG. 16E, and FIG. 16F show the ultra-structure characterization and anti-Aβ immuno-gold labeling of selected plaques in the precuneus. FIG. 16A Scanning electron microscope image of the Pt2 plaque (1,248×) in the precuneus (from FIG. 15B). Scale bar, 40 μm. FIG. 16B Transmission electron microscope image of the same plaque (1,500×). Scale bar, 2 μm. Upper right Inset is a 12,000× magnification of the region outlined in red. FIG. 16C High magnification (25,00×) of the region outlined in red in FIG. 16B, red arrows heads highlight 10 nm gold particles after immuno-labeling with anti-Aβ antibody 82E1. Scale bar, 100 nm. FIG. 16D Scanning electron microscope image of the Pt2 plaque (1,250×) in the precuneus (from FIG. 15H). Scale bar, 40 μm. FIG. 16E Transmission electron microscope image of the same plaque (1,500×). Scale bar, 2 μm. FIG. 16F High magnification (30,00×) of the region outlined in red in FIG. 16E, red arrows heads highlight fibrils. Scale bar, 100 nm.

FIG. 17A, FIG. 17B, FIG. 17C and FIG. 17D show quantitative NanoSIMS imaging of region 50 μm away from plaque of Pt2. FIG. 17A Scanning electron microscope image (1,248×) of the plaque in FIG. 15A-B. Scale bar, 40 μm; red rectangle outlines region near the plaque imaged by NanoSIMS. FIG. 17B NanoSIMS ion map of ¹²C¹⁴N— in a chained analysis taken near the plaque at 25×45 μm. Scale bar, 2 μm. FIG. 17C All eight images were subdivided into 625 ROIs each representing 10×10 pixels from which carbon ratios were calculated. The histogram plots all ROIs and their respect ¹³C¹⁴N/¹²C¹⁴N ratios. Blue line is the theoretical natural abundance of ¹³C, red line the measured ¹³C¹⁴N/¹²C¹⁴N ratio of an unlabeled AD brain with ±standard deviation in orange, and the mean of the unlabeled sample+2σ in green. FIG. 17D ¹³C¹⁴N/¹²C¹⁴N image showing the distribution of ¹³C in the sample per pixel; each pixel is the width of the Cs+ beam—100 nm. ROIs that were significantly enriched in ¹³C as described in the Methods and Materials are outline in red.

FIG. 18A, FIG. 18B, FIG. 18C, and FIG. 18D show targeted nLC-MS/MS of Aβ proteoforms from an insoluble fraction of SILK participant 2. Extracted ion chromatograms (XIC) are shown for FIG. 18A Aβ mid-domain, FIG. 18B Aβx-40, and FIG. 18C Aβx-42 transitions in participant 2 (8 day delta between labeling and expiration) shown on the top row as unlabeled, ¹⁵N internal standard (Heavy), and ¹³C₆-leucine labeled (SILK). Below each Aβ proteoform from Pt2 are the XICs from an unlabeled AD participant used to calculate isotopic background. FIG. 18D The average of each labeled/unlabeled ratio from triplicate injections using targeted nLC-MS/MS. Error bars represent ±SD.

FIG. 19A, FIG. 19B, FIG. 19C, and FIG. 19D show the analysis of carbon imaging. FIG. 19A-FIG. 19D a subsection of a plaque (20 μm) was image by NanoSIMS with four electron multipliers set to detect 12C14N—, 12C, 13C14N—, and 13C, respectively. Apparent from FIG. 19A and FIG. 19C is the improved image morphology and topology when carbon is imaged as a cyanide ion (i.e., a molecule) compared to carbon isotopes alone; FIG. 19B and FIG. 19D. We hypothesize that this may be due to the higher ionization potential, of cyanide ions compared to carbon as evidenced by the signal intensity (Cts).

FIG. 20A, FIG. 20B, FIG. 20C, and FIG. 20D show labelled (100%) B-cell Hybridoma. FIG. 20A-FIG. 20B ¹²C¹⁴N— and ¹³C¹⁴N— isotope maps (50 μm). FIG. 20C Ratio of ¹³C¹⁴N—/¹²C¹⁴N— at same section as FIG. 20A-FIG. 20B with ¹³C enrichment the ratio is above natural abundance. FIG. 20D Image of the standard deviation of ¹³C¹⁴N—/¹²C¹⁴N— ratios relative to natural abundance. The significance plot, generated by Poisson statistics, is a visual way to see statistical significance or deviation from the natural abundance of ¹³C¹⁴N—/¹²C+N— ratio.

FIG. 21 shows a standard curve showing the ¹³C¹⁴N—/¹²C¹⁴N— ratio over the percent of ¹³C₆-Leucine label.

FIG. 22A, FIG. 22B, FIG. 22C, FIG. 22D, FIG. 22E, FIG. 22F and FIG. 22G show data from the a frontal lobe. FIG. 22A Plaque optical image (32×). FIG. 22B-FIG. 22C Images (40 μm) of plaque with ¹²C¹⁴N— and ¹³C¹⁴N— isotopes, respectively. FIG. 22D Ratio of ¹³C¹⁴N—/¹²C¹⁴N—. Mean ratio of ¹³C¹⁴N—/¹²C¹⁴N— for ROIs 1-10 and their errors were normalized to the non-labeled standard and calculated in quadrature. The data suggests that the ratio is 1.31%±0.05%. This indicates 18% increase above the natural abundance of ¹³C/¹²C, though more data with appropriate negative controls needs to be collected. FIG. 22E Isotopic map of ³¹P. FIG. 22F Co-localization of carbon (red, ¹²C¹⁴N—) and phosphorus (green, ³¹P) indicates phospholipids of cellular membranes. FIG. 22G Higher resolution re-measure of sub-area (green box) was performed at 15 μm to crosscheck for a stochastic false positive.

FIG. 23A, FIG. 23B, FIG. 23C, FIG. 23D, FIG. 23E and FIG. 23F is an image of NanoSIMS imaging of unlabeled human brain. FIG. 23A Secondary electron images in the mode where negative secondary ions are analyzed. FIG. 23B SIMS analysis of the same section at mass ¹²C—. FIG. 23C The same section at mass ¹³C—. FIG. 23D Ratio of ¹³C—/¹²C— at same section; as expected, without ¹³C enrichment (e.g., SILK) the ratio is at natural abundance. FIG. 23E ¹²C-image of a nearby region; the image demonstrates myelination (circles) by oligodendrocytes. Myelin, a lipid, is abundant in carbon compared to proteins. Yellow points of high ¹²C enrichment indicate nuclei. FIG. 23F ¹⁶O— image of the same in FIG. 23E. Quantitative atomic mass images of analyzed surface reveals the distribution of isotopes, which allows visualization of the morphology of the sample. C/s; counts/second. Scale bar: 5 μm.

FIG. 24 shows a model of plaque growth and SILK. Core (blue) is abundant in ¹²C, while the ¹³C-labeled proteins (purple) are deposited around the core. Finally, after labeling, ¹²C proteins are deposited once more (blue). Arrows indicate lateral circumferential growth s.p.; senile plaque.

FIG. 25 shows plaques in entorhinal cortex from 3 participants. Each circle represents an individual plaque. Horizontal bars are median % ¹⁵N/¹⁴N. Green line, positive control—mouse with 24 hr data±s.d. Black line, negative control—unlabed human brain±s.d. CDR, clinical dementia rating.

FIG. 26 shows neuronal metabolism. Left: Neurons in the entorhinal cortex from 3 participants. Each circle represents an individual neuron. Horizontal bars are median % ¹⁵N/¹⁴N. Green line, positive control & black line, negative control as in FIG. 25. Right: A neuron from Pt1 (top row) and Pt6 (bottom row). ¹⁵N/¹⁴N images show ¹⁵N label in the neuron. ¹²C¹⁴N carbon image for detail. CDR, clinical dementia rating.

FIG. 27A, FIG. 27B, and FIG. 27C show ¹³C— VS ¹⁵N-Spirulina by SILK-SIMS. To optimize the acquisition time, we tested two labeling protocols in 6-month-old male APP/PS1 mice treated with commercially available ¹⁵N— or ¹³C-labeled Spirulina from Cambridge Isotope Laboratories. FIG. 27A) Mice received a single oral dose (0.5 grams) of either ¹⁵N-Spirulina or ¹³C-Spirulina, in their water (ad libitum) and were returned to normal drinking water 12 hours later. Subsequently, the treated mice were sacrificed at specific time intervals. FIG. 27B) SILK-SIMS imaging revealed that ¹⁵N was enriched in brain tissue at 4 weeks after the single oral dose. FIG. 27C) By contrast, ¹³C label rapidly diminished at 24 hours after labeling. Therefore, ¹⁵N has substantially longer half-life in brain tissue than ¹³C. The use of ¹⁵N-Spirulina has the following advantages: (i) it is inexpensive (ii) the heavy isotope signal can be increased to reduce SILK-SIMS data acquisition times and background error; (iii) the ¹⁵N signal is not affected by the use of carbon-rich embedding resin in fixed specimens, which can affect the ¹³C signal by reducing the measured ¹³C levels below natural abundance (FIG. 2C); and (iv) ¹⁵N has a natural abundance of 0.37% (for every ¹⁵N atom there are 272 ¹⁴N atoms), which generates less background noise then for ¹³C measurements.

FIG. 28 shows an illustration infusion of ¹³C₆-leucine in a breast cancer subject. After removal or biopsy the tumor is processed for NanoSIMS imaging. Data will be used to determine how aggressive the cancer is and is used to make determinations on how the subject should be treated or determine how the subject is responding to treatment.

FIG. 29 shows hospice study workflow. MD, medical doctor, NP, nurse practitioner; SW, social worker; AD8, eight question assessment (ref#52,53), MoCA, Montreal Cognitive Assessment (ref#51); CDR (ref#54,55); ToD, time of death; Vent. CSF, ventricular cerebral spinal fluid.

Those of skill in the art will understand that the drawings, described above, are for illustrative purposes only. The drawings are not intended to limit the scope of the present teachings in any way.

### DETAILED DESCRIPTION

Provided herein are methods and systems for detecting and modeling the in vivo kinetics, localization and metabolism of a biomolecule or therapeutic agent, related to the health/disease state of a subject or treatment thereof. The present disclosure is based, at least in part, on the discovery that coupling NanoSIMS with SILK (the new method coined stable isotope labeled kinetics-secondary ion mass spectrometry (SILK-SIMS)) can spatially and quantitatively analyze biomolecule dynamics, including for example kinetics and metabolism in vivo. As shown herein, it has been demonstrated that the disclosed systems and methods are effective in being able to localize and quantify the stable, non-radioactive isotope ¹³C in cells, mouse brains, and the human brain.

NanoSIMS is an important and routinely employed analytical method for investigating isotopic compositions in the fields of Material Science, Cosmochemistry, and Geochemistry; however, this technique remains under-utilized in the Biological and Biomedical Sciences. In the disclosed technique, a focused primary ion beam is electrostatically scanned across the tissue producing secondary ions. These secondary ions are transmitted through ion optics (similarly to visible light in microscopes, but using electrostatic lenses) for mass separation and detection of elemental isotopes, which can be measured to generate a quantitative spatial profile of biological tissue. By analogy this technology is akin to PET imaging, but at the isotopic level and with ultra-high sensitivity and spatial resolution (100 nm).

As described herein, this analytical method has been coupled to in vivo SILK to spatially and quantitatively quantify deposition of newly synthesized protein into amyloid plaques in AD brain in a method coined stable isotope labeled kinetics-secondary ion mass spectrometry (SILK-SIMS). Furthermore, the first metabolic images of plaque growth in both human and mouse brain were generated. This finding can open an entire field in aggregated protein research and ultrastructural metabolic and kinetic rates in the nervous system. This discovery has led and accelerated the launch of a clinical study to label patients in hospice before death and brain donation with the first measures of Alzheimer's disease metabolic growth of plaques measured at the nanometer scale. As such, the disclosed methods and systems can generate profound insights into mechanisms of physiology, metabolism and kinetics of a variety of health/disease states that will be useful for development of tests and treatments through target validation studies.

This invention can be useful as a tool for measuring the metabolism and determining the site localization of biomolecules (e.g., lipids, proteins, peptides, carbohydrates) and therapeutic agent (e.g., pharmaceutical agents or scheduled drugs) in biological samples at nanometer resolution. This invention provides precise sub-cellular localization and metabolism of compounds in vivo. This comes with the added benefit that sample perturbations engendered by staining or genetic label incorporation are avoided.

This invention is currently applied to Alzheimer's disease research, but can be translated to other aspects of neurodegenerative disease, such as Parkinson's disease, and other diseases such as cancer, heart disease, and diabetes (not inclusive) to expedite development of treatments. The disclosed methods and systems can contribute to the advancement in the understanding physiology and pathophysiology.

The disclosed methods and systems can be used as a diagnostic tool or be used to detect prognostic indicators of disease. Suitable compositions and methods of the invention are discussed in more detail below.

### (I) Methods of Detecting the In Vivo Localization and Molecular Kinetics of a Biomolecule or Therapeutic Agent

The present disclosure provides methods, integrating NanoSIMS with clinically-accepted in vivo metabolic labeling of tissue with an isotope label (SILK-SIMS) that can generate kinetic data, including images, of biological processes. For example, when applied to a tumor, the present methods can reveal heterogeneous spatial distributions of newly synthesized versus pre-existing lipids, with altered molecular flux patterns distinguishing region-specific intra-tumor subpopulations or calculate the rate of glucose consumption as a measure of the aggressiveness of the tumor. In the context of AD, SILK-SIMS can reveal the quantity and kinetics of Aβ plaque deposition in human AD brain. This approach can characterize the diversity of molecular flux across heterogeneous tissue, enable identification of specific molecules involved in metabolism of region-specific cell subpopulations and enable identification of resistance mechanisms to drugs.

Functional dynamic processes may be imaged along spatial coordinates in tissue histopathology specimens in the present methods. The concept of SILK-SIMS and the resulting metabolic flux histopathology images, is analogous to traditional static microscopy, such as vital dyes, in situ hybridization histochemistry, immunohistochemistry or electron microscopy. In these traditional static microscopies, dye-binding molecules, mRNA transcripts, protein antigens or electron-scattering structures, respectively, are visualized and mapped in a tissue. In the present disclosure, the dynamic metabolic fluxes of biomolecules or therapeutic agents and metabolic pathways, rather than their structure or concentration are detected, localized, imaged, and quantified.

“Metabolic fluxes” or “molecular kinetics” are defined as the rates of chemical transformation or spatial movement of molecules and their flow through reactions and pathways in the metabolic network of a living system. “Flux(es)” are by definition rates (motion, in space or time), as contrasted with statics (snapshots of molecules at rest, lacking the motion and the dimension of time). Metabolic fluxes can refer to kinetics of small molecules, polymers, or macromolecules. Fluxes or rates of metabolic processes that can be imaged in a tissue and include, in non-limiting examples, protein/protein aggregation, synthesis, degradation, oxidation, reduction, methylation, polymerization, conjugation, addition, condensation, cleavage, re-arrangement, and other chemical reactions, as well as physical movement in space including transport, accessibility, storage, secretion, uptake, or other dynamic processes occurring in a living organism.

Provided herein are methods for detecting the in vivo localization, molecular kinetics of a biomolecule. In one aspect, the biomolecule is one or more amyloid-β (Aβ) isoforms. In another aspect the biomolecule is a cancer related biomolecule. As used herein, the term “biomolecule” refers to a substance in a biological sample that may be measured as an indication of the health/disease state of a subject. For instance, a biomolecule may be a protein (e.g. a chemokine, an antibody, or other protein), a carbohydrate or carbohydrate moiety (e.g. a sugar, a starch, or a proteoglycan), a lipid or lipid moiety, a nucleotide or nucleotide sequence, or other biomolecule.

Provided herein are methods for detecting the in vivo localization, molecular kinetics and metabolism of an isotope labeled therapeutic agent (e.g., pharmaceutical agents or scheduled drugs). As used herein, the phrase “therapeutic agent” is intended to have its broadest possible interpretation and refers to any therapeutically active substance that is delivered to a bodily conduit of a living being to produce a desired, usually beneficial, effect. More particularly, a therapeutic agent relates to any agent that can confer a therapeutic benefit on a patient and includes, without limitation, conventional drugs, gene therapy constructs, chemotherapeutic agents, antibiotics, macromolecules, protein bound drugs, cell-based therapies such as using bone marrow-derived mesenchymal stem cells, oncolytic virus (e.g. Delta-24), fractions of tissues or cells, nanoparticles, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals. Exemplary therapeutic agents include analgesics, anesthetics, anxiolytics, antidepressants such as selective serotonin reuptake inhibitors like citalopram, escitalopram oxalate, fluoxetine, fluvoxamine maleate, paroxetine, sertraline, and dapoxetine, antipsychotics including clozabine, risperidone, olanzapine, quetiapine, ziprasidone, aripiprazole, paiperidone, sertindole, zotepine, amisulpride, and melperone, and olanzapine, anticonvulsants, nervous system stimulants, antiemetics, hallucinogens, mood stabilizers, bronchodilators, decongestants, anti-proliferatives, angiotensin converting enzyme inhibitors, antiarrhythmics, antianginals, antihypertensives, antihyperlipidemics including, for example, any of a number of statin drugs such as atorvastatin, cerivastatin, fluvastatin, lovastatin, mevastatin, pitavastatin, pravastatin, rosuvastatin, simvastatin, and ezetimibe with simvastatin, anticoagulants such as warfarin, acenocoumarol, phenprocoumon and phenindione, antiplatelets, beta blockers, diuretics, thrombolytics, vasodilators, antacids, antidiarrheals, H2-receptor antagonists, proton pump inhibitors, laxatives, anti-inflammatories, antirheumatics, corticosteroids, muscle relaxants, anti-histamines, antibiotics, anti-virals such as ribavirin, ganciclovir, abacavir, tenofovir, vidarabine, emtricitabine, efavirenz, darunavir, delavirdine, nevirapine, protease inhibitors, lopinavir, zalcitabine, didanosine, seliciclib, chloroquine, resveratrol, and zidovudine, vaccines, anti-protozoals, anti-fungals, antihelmintics, anti-diabetics including sulfonylureas such as tolbutamide, acetohexamide, tolazamide, chlorpropamide, glipizide, glyburide glimepiride, and gliclazide, meglitinides, biguanides such as metformin, glitazones such as rosiglitazone, pioglitazone, and troglitazone, alpha glucosidase inhibitors such as miglitol and acarbose, and DPP-4 inhibitors such as vildagliptin and sitagliptin, and chemotherapeutics which include agents such as paclitaxel, doxorubicin, and other drugs which have been known to affect tumors. Chemotherapeutics, as used herein, further includes agents which modulate other states which are related to tissues which can be permeabilized using the methods and compositions of the invention. The chemotherapeutic agent can be, for example, a steroid, an antibiotic, or another pharmaceutical composition. Examples of chemotherapeutic agents include agents such as paclitaxel, doxorubicin, vincristine, vinblastine, vindesine, vinorelbin, taxotere (DOCETAXEL), topotecan, camptothecin, irinotecan hydrochloride (CAMPTOSAR), doxorubicin, etoposide, mitoxantrone, daunorubicin, idarubicin, teniposide, amsacrine, epirubicin, merbarone, piroxantrone hydrochloride, 5-fluorouracil, methotrexate, 6-mercaptopurine, 6-thioguanine, fludarabine phosphate, cytarabine (ARA-C), trimetrexate, gemcitabine, acivicin, alanosine, pyrazofurin, N-Phosphoracetyl-L-Asparate (PALA), pentostatin, 5-azacitidine, 5-Aza-2′-deoxycytidine, adenosine arabinoside (ARA-A), cladribine, ftorafur, UFT (combination of uracil and ftorafur), 5-fluoro-2′-deoxyuridine, 5-fluorouridine, 5′-deoxy-5-fluorouridine, hydroxyurea, dihydrolenchlorambucil, tiazofurin, cisplatin, carboplatin, oxaliplatin, mitomycin C, BCNU (Carmustine), melphalan, thiotepa, busulfan, chlorambucil, plicamycin, dacarbazine, ifosfamide phosphate, cyclophosphamide, nitrogen mustard, uracil mustard, pipobroman, 4-ipomeanol, dihydrolenperone, spiromustine, geldanamycin, cytochalasins, depsipeptide, Lupron, ketoconazole, tamoxifen, goserelin (Zoledax), flutamide, 4′-cyano-3-(4-fluorophenylsulphonyl)-2-hydroxy-2-methyl-3′-(trifluorometh-yl)propionanilide, Herceptin, anti-CD20 (Rituxan), interferon alpha, interferon beta, interferon gamma, interleukin 2, interleukin 4, interleukin 12, tumor necrosis factors, and radiation. Representative compounds used in cancer therapy further include cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, dunorubicine, epirubicine, bleomycin, nitomycin, cisplatin, carboplatin, procarbazine, amacrine, mitoxantron, tamoxifen, nilutamid, and aminoglutemide. Further included within the meaning of “therapeutic agents” are immuno-suppressants, hormonal contraceptions, selective estrogen receptor modulators, fertility agents, and anti-pruritics. The therapeutic agent may be formulated as microparticles or nanoparticles. Other examples of therapeutic agents include macromolecules, such as, liposomes, nanoparticles, plasmid, viral vectors, non-viral vectors, and oligonucleotides.

Therapeutic agents encompass numerous chemical classes, for example, organic molecules, such as small organic compounds having a molecular weight of more than 50 and less than about 2,500 Daltons. Therapeutic agents can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A therapeutic agent can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Therapeutic agents for use according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

The present methods may include a kinetic model developed and/or calibrated utilizing measured data from subjects. This disclosure further provides developing a model by determining and predicting steady state molecular kinetic parameters. Also provided are methods for using the model to identify a subject's health/disease state. The method of developing the model may include, but is not limited to, measuring a concentration of a labeled moiety introduced into a subject over a period of time. The labeled moiety may be incorporated into a biomolecule, a biomolecule precursor, or therapeutic agent within the subject or may be incorporated into a biomolecule, a biomolecule precursor, or therapeutic agent and administered to the subject. The method may further include measuring concentrations in a biological sample of the biomolecule or therapeutic agent incorporating the isotope label in the subject, and incorporating the measured data into known or hypothesized relationships and/or metabolic processes. In an aspect, the method may comprise developing a model which may predict the measured values. The model may be developed by calibrating the predicted values against measured values and adjusting a set of model parameters to provide a best fit of the predicted molecular kinetics of the one or more labeled biomolecules or therapeutic agent to the measured kinetics from the subject. In an aspect, the model may output model parameters specific for each subject.

The concentrations of the one or more labeled biomolecules or therapeutic agent may be collected in a specific region of interest within the biological sample. For example, the concentrations of the one or more labeled biomolecules or therapeutic agent may be measured in a particular cell or within a specific sub-cellular region (e.g. a cellular organelle or Aβ plaque). The concentrations of the one or more labeled biomolecules or therapeutic agent and associated metabolic processes in the biological sample may be represented within the model. In one aspect, this representation within the model may include a compartment, a rate constant, flow equation, and/or any other mathematical representation known in the art without limitation. In an aspect, the concentration in a compartment may be calculated by multiplying the concentration in the previous compartment by a transfer rate constant between the two compartments minus any irreversible loss. Different aspects of the model may be differentiated by different numbers of compartments or types of compartments, the order of the compartments, the equations governing the trafficking and flow of biomolecules/therapeutic agent or any other aspect for modeling the molecular kinetics of a biomolecule or therapeutic agent.

In another aspect, the methods may detect the movement of a biomolecule or therapeutic agent within the subject. In an aspect, the concentration of a labeled moiety and measured concentrations of labeled biomolecule or therapeutic agent in the biological sample may be used to develop a model of the molecular kinetics of the labeled biomolecule or therapeutic agent and to determine the rate constants associated with each compartment or flow equation. In addition, the model may be used to calculate predicted concentrations of the biomolecule or therapeutic agent, in the brain, in the tumor, or at any other location in a subject. Non-limiting examples of how the model of the biomolecule or therapeutic agent molecular kinetics may be used include identifying the health/disease state of a subject, fitting a curve of measured data acquired from a subject, predicting the metabolism, processing, and/or concentration of the biomolecule or therapeutic agent in a subject, identifying sensitive pathway components to help design treatments or understand a disease, and investigating changes in the kinetics of the biomolecule or therapeutic agent that may be induced by physiological, pathophysiological, or treatment conditions.

### (a) Isotope Labeling

The isotope label may be any known stable isotope or radioisotope. For example, without limitation, the stable isotope may include ²H, ¹³C, ¹⁵N, ¹⁸O, ¹⁷O, ³H, ¹⁴C, ³⁵S, ³²P, ¹²⁵I, ¹³¹I, ¹⁹F and ⁸¹Br or other isotopes of elements present in organic systems. In one embodiment, the stable isotope is ¹³C.

In some embodiments, the labeled moiety may be any molecule or combination of molecules having an isotope label that is incorporated into a biomolecule or therapeutic agent. Isotope labels may be used to modify all precursor molecules disclosed herein to form isotope-labeled precursors. The entire precursor molecule may be incorporated into one or more biomolecules or therapeutic agents. Alternatively, a portion of the precursor molecule may be incorporated into one or more biomolecules or therapeutic agents. Precursor molecules may include without limitation, for example, CO₂, NH₃, glucose, lactate, H₂O, acetate, and fatty acids. A precursor molecule used may be any precursor molecule known in the art for a specific incorporation into a biomolecule or therapeutic agent. In some embodiments, the precursor molecule is incorporated into a therapeutic agent during the synthetic chemical processing of the therapeutic agent.

A protein precursor molecule may be any protein precursor molecule known in the art. These precursor molecules may be CO₂, NH₃, glucose, lactate, H₂O, acetate, and fatty acids. Precursor molecules of proteins may also include one or more amino acids. The precursor may be any amino acid. The precursor molecule may be a singly or multiply deuterated amino acid. For example, the precursor molecule may be selected from ¹³C-lysine, ¹⁵N-histidine, ¹³C-serine, ¹³C-glycine, ²H-leucine, ¹⁵N-glycine, ¹³C-leucine, ²H₅-histidine, ¹³C₆-phenylalanine and any deuterated amino acids. In some embodiments, the amino acid is labeled with multiple isotopes. Labeled amino acids may be administered, for example, undiluted or diluted with non-labeled amino acids. All isotope labeled precursors may be purchased commercially, for example, from Cambridge Isotope Labs (Andover, Mass.). Generally, the choice of amino acid is based on a variety of factors such as: (1) the amino acid generally is present in at least one residue of the protein or peptide of interest; (2) the amino acid is generally able to quickly reach the site of protein synthesis and rapidly equilibrate to a region of interest; (3) the amino acid ideally may be an essential amino acid (not produced by the body), so that a higher percent of labeling may be achieved; (4) the amino acid label generally does not influence the metabolism of the protein of interest (e.g., very large doses of leucine may affect muscle metabolism); and (5) the relatively wide availability of the desired amino acid (i.e., some amino acids are much more expensive or harder to manufacture than others).

In an aspect, the amino acid leucine may be used to label proteins that are synthesized in the CNS. Non-essential amino acids may also be used; however, measurements may be less accurate. In one aspect, ¹³C₆-phenylalanine, which contains six ¹³C atoms, may be used to label a neurally derived protein. In an aspect, ¹³C₆-leucine may be used to label a neurally derived protein. In an exemplary aspect, ¹³C₆-leucine may be used to label amyloid-β.

There are numerous commercial sources of labeled amino acids, containing both non-radioactive isotopes and radioactive isotopes. Generally, the labeled amino acids may be produced either biologically or synthetically. Biologically produced amino acids may be obtained from an organism (e.g., kelp/seaweed) grown in an enriched mixture of ¹³C, ¹⁵N, or another isotope that is incorporated into amino acids as the organism produces proteins. The amino acids are then separated and purified. Alternatively, amino acids may be made using any known synthetic chemical processes.

Protein precursor molecules may also include any precursor for post-translational or pre-translationally modified amino acids. These precursors include, for example, precursors of methylation such as glycine, serine or H₂O; precursors of hydroxylation, such as H₂O or O₂; precursors of phosphorylation, such as phosphate, H₂O or O₂; precursors of prenylation, such as fatty acids, acetate, H₂O, ethanol, ketone bodies, glucose, or fructose; precursors of carboxylation, such as CO₂, H₂O, O₂, or glucose; precursors of acetylation, such as acetate, ethanol, glucose, fructose, lactate, alanine, H₂O, O₂, or CO₂; and other post-translational modifications known in the art.

The degree of labeling present in free amino acids may be determined experimentally, or may be assumed based on the number of labeling sites in an amino acid. For example, when using hydrogen isotopes as a label, the labeling present in C—H bonds of free amino acid or, more specifically, in tRNA-amino acids, during exposure to ²H₂0 in body water may be identified. The total number of C—H bonds in each non-essential amino acid is known—e.g. 4 in alanine, 2 in glycine, etc.

### (b) Administration

The methods of administering the one or more isotope-labeled precursors, labeled biomolecules or labeled therapeutic agent may vary depending upon the absorptive properties of the isotope-labeled moiety and the specific biosynthetic pool into which each compound is targeted. Isotope-labeled moieties may be administered to organisms, plants and animals including humans directly for in vivo analysis. In addition, isotope-labeled moieties may be administered in vitro to living cells. Specific types of living cells include hepatocytes, adipocytes, myocytes, fibroblasts, neurons, pancreatic β-cells, intestinal epithelial cells, leukocytes, lymphocytes, erythrocytes, microbial cells and any other cell-type that can be maintained alive and functional in vitro.

Generally, an appropriate mode of administration is one that produces a steady state level of the isotope-labeled moiety within the biosynthetic pool and/or in a reservoir supplying such a pool for at least a transient period of time. The isotope-labeled moiety may be administered to a subject using any one of at least several methods known in the art. Non-limiting examples of suitable methods of administration include intravenous, intra-arterial, subcutaneous, intraperitoneal, intramuscular, and oral administration. In one aspect, the labeled moiety is administered to the subject using intravenous infusion. In some embodiments, the labeled moiety is administered as an IV bolus. In some embodiments, the labeled moiety is administered by oral administration.

The labeled moiety may be administered slowly over a period of time or as a large single dose depending upon the type of analysis chosen (e.g., steady state or bolus). To achieve steady-state levels of the labeled biomolecule, the labeling time generally should be of sufficient duration so that the labeled biomolecule may be reliably quantified. This duration may be selected to be sufficient to result in saturation of the biochemical pathways associated with the synthesis of the biomolecule. In one aspect, the duration may be sufficient to result in the saturation of the biochemical pathways associated with the synthesis and kinetics of at least one Aβ isoform in the brain of a subject, including, but not limited to: APP synthesis, cleavage of C99 and the at least one Aβ isoform, the transport of the at least one Aβ isoform to the CSF, and the transport of the at least one Aβ isoform to the blood. In another aspect, the saturation of the biochemical pathways may be indicated by the detection of stabilized levels of the at least one Aβ isoform in the CSF and/or blood as measured in a patient. In another aspect, the duration may be sufficient to result in the saturation of the biochemical pathways associated cancer development, tumor progression or apoptosis.

In an aspect, the labeled moiety is administered intravenously for an amount of time that is less than the half-life of the biomolecule or therapeutic agent in a biological sample. In other aspect, the labeled moiety is administered intravenously for an amount of time that is greater than the half-life of the biomolecule or therapeutic agent in a biological sample. For example, the labeled moiety may be administered intravenously over a duration of minutes to hours, including, but not limited to, for at least 10 minutes, at least 20 minutes, at least 30 minutes, at least 1.0 hour, at least 1.5 hours, at least 2.0 hours, at least 2.5 hours, at least 3.0 hours, at least 3.5 hours, at least 4.0 hours, at least 4.5 hours, at least 5.0 hours, at least 5.5 hours, at least 6.0 hours, at least 6.5 hours, at least 7.0 hours, at least 7.5 hours, at least 8.0 hours, at least 8.5 hours, at least 9.0 hours, at least 9.5 hours, at least 10.0 hours, at least 10.5 hours, 1 at least 1.0 hours, at least 11.5 hours, or at least 12 hours. In another aspect, the labeled moiety may be administered intravenously over a period ranging from about 6 hours to about 18 hours. In another aspect, the labeled moiety may be administered intravenously over a period of about 9 hours. In another aspect, the labeled moiety may be administered intravenously over a period of about 3 hours. In yet another aspect, a labeled moiety is administered orally as multiple doses. The multiple doses may be administered sequentially or an amount of time may elapse between each dose. The amount of time between each dose may be a few seconds, a few minutes, or a few hours. In each of the above embodiments, the labeled moiety can be labeled leucine, labeled phenylalanine, or any other labeled amino acid that is capable of crossing the blood brain barrier.

Those of skill in the art will appreciate that the amount (or dose) of the labeled moiety can and will vary. Generally, the amount is dependent on (and estimated by) the following factors. (1) The type of analysis desired. For example, to achieve a steady state of about 15% labeled leucine in plasma requires about 2 mg/kg/hr over 9 hr after an initial bolus of about 3 mg/kg over 10 min. In contrast, if no steady state is required, a bolus of labeled leucine (e.g., about 400 mg to about 800 mg of labeled leucine) may be given. (2) The biomolecule or therapeutic agent under analysis. For example, if the Aβ variant is being produced rapidly, then less labeling time may be needed and less label may be needed—perhaps as little as 100 mg or less as a bolus. And (3) the sensitivity of the technology to detect label. For example, as the sensitivity of label detection increases, the amount of label that is needed may decrease.

In another aspect, a labeled moiety is administered orally as a single bolus. In another aspect, a labeled moiety is administered intravenously as a single bolus. In another aspect, a labeled moiety is administered on multiple days. In still another aspect, a labeled moiety is administered intravenously as an infusion for about 1 hour. All three methods of administration (oral bolus, IV bolus, and IV infusion) work equally well in terms of providing a reliable measure of amyloid beta metabolism. An intravenous bolus of a labeled moiety and an oral bolus of labeled moiety are easier to administer than an intravenous infusion, and also results in maximal levels of free label at an earlier time point (e.g. about 5 to about 10 minutes, and about 30 to about 60 minutes, respectively, for labeled leucine). In each of the above embodiments, the labeled moiety can be labeled leucine, labeled phenylalanine, or any other labeled amino acid that is capable of crossing the blood brain barrier.

### (c) Biological Sample

The biological sample used in the methods described herein may be obtained from a living or deceased subject and then prepared as described herein. In some embodiments, at least 1, at least 2, at least 3, at least 4, at least 5, at least 6, at least 7, at least 8, at least 9, at least 10 or more biological samples may be obtained from the same subject and analyzed by SILK-SIMS.

A biological sample may include a tissue histology specimen from tissues such as, for example, skin, organs, breast, prostate, brain, bone, muscle, liver, and gut. The sample may also be obtained from bodily fluids including, for example, urine, blood, interstitial fluid, edema fluid, saliva, lacrimal fluid, inflammatory exudates, synovial fluid, abscess, empyema or other infected fluid, cerebrospinal fluid, sweat, pulmonary secretions (sputum), seminal fluid, feces, bile, and intestinal secretions. The biological sample may further include biofilms, microbiomes and other microbial organisms. The biological sample may be a clinical sample, upon which a clinical decision, diagnosis or prognosis can be made using the output generated according to the methods described herein.

The biological sample may be obtained, for example, by blood draw, urine collection, biopsy, or other methods known in the art. In some embodiments, the sample is obtained by taking a surgical biopsy; surgical removal of a tissue or portion of a tissue; performing a percutaneous, endoscopic, transvascular, radiographic-guided or other non-surgical biopsy; euthanizing an experimental animal and removing tissue; collecting ex vivo experimental preparations; removing tissue at post-mortem examination; or other methods known in the art for collecting tissue samples. The methods of obtaining a sample may also vary and be specific to the molecules of interest. In some embodiments the biological sample is a cancer or tumorous tissue.

Standard techniques for preparing a biological sample for nanoSIMS include, for example, freezing and slicing, lyophilization, cryopreservation, ethanol dehydration, OCL preservation, and other suitable methods known in the art. In some embodiments, the samples are prepared on a slide with a coated surface that permits or increases energy-dependent volatilization of molecules from the surface of the slide. In some embodiments, the techniques for preparing a biological sample include those as described in the below examples.

### (d) Subject

A suitable subject includes a human, a livestock animal, a companion animal, a lab animal, or a zoological animal. In one embodiment, the subject may be a rodent, e.g., a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

In some embodiments, the subject may be diagnosed with a disease or disorder or may be suspected of having a disease or disorder (e.g. heart disease or diabetes). In an aspect, a subject may suffer from or be suspected of suffering from Aβ amyloidosis. The term “Aβ amyloidosis’ refers to Aβ deposition in a subject that may result from differential metabolism (e.g. increased production, reduced clearance, or both). Aβ amyloidosis is clinically defined as evidence of Aβ deposition in the brain either by amyloid imaging (e.g. PiB PET) or by decreased cerebrospinal fluid (CSF) Aβ42 or Aβ42/40 ratio. See, for example, Klunk W E et al. Ann Neurol 55(3) 2004, and Fagan A M et al. Ann Neurol 59(3) 2006, each hereby incorporated by reference in its entirety. Subjects with Aβ amyloidosis are also at an increased risk of developing a disease associated with Aβ amyloidosis. Diseases associated with Aβ amyloidosis include, but are not limited to, Alzheimer's Disease (AD), cerebral amyloid angiopathy, Lewy body dementia, and inclusion body myositis. Non-limiting examples of symptoms associated with Aβ amyloidosis may include impaired cognitive function, altered behavior, abnormal language function, emotional dysregulation, seizures, dementia, and impaired nervous system structure or function.

In another aspect, a subject may suffer from or be suspected of suffering from a degenerative disease. Any degenerative disease characterized by the dysregulation in the turnover and production rate of any biomolecule including, but not limited to at least one Aβ isoform may be predicted using the present methods without limitation. By way of non-limiting example, Alzheimer's Disease (AD) is a debilitating disease characterized by accumulation of amyloid plaques in the central nervous system resulting from increased production, decreased clearance, or a combination of increased production and decreased clearance of Aβ protein. While AD is an exemplary disease that may be diagnosed or monitored by various aspects of this disclosure, this disclosure is not limited to AD. It is envisioned that the method may be used in modeling the kinetics, diagnosis, and assessment of treatment efficacy of several neurological and neurodegenerative diseases, disorders, or processes including, but not limited to, AD, Parkinson's Disease, stroke, frontal temporal dementias (FTDs), Huntington's Disease, progressive supranuclear palsy (PSP), corticobasal degeneration (CBD), aging-related disorders and dementias, Multiple Sclerosis, Prion Diseases (e.g. Creutzfeldt-Jakob Disease, bovine spongiform encephalopathy or Mad Cow Disease, and scrapie), Lewy Body Disease, and Amyotrophic Lateral Sclerosis (ALS or Lou Gehrig's Disease). It is also envisioned that the method of modeling in vivo kinetics of a CNS disease may be used to study the normal physiology, metabolism, and function of the CNS.

In some embodiments, the present methods may be used to detect one or more biomolecules or therapeutic agents in a biological sample from a subject with a tumor or cancer. A tumor or cancer refers to a condition usually characterized by unregulated cell growth or cell death. A tumor may be malignant when nearby tissues or other parts of the body are invaded by the tumor. A tumor may be traditionally treated by surgical resection, radiation therapy, or chemotherapy. Any cancers or tumors, including both malignant and benign tumors as well as primary tumors and metastasis may comprise the biological sample as disclosed herein. In a specific embodiment, the disclosure provides method to detect a biomolecule or therapeutic agent in a cancer wherein the cancer is any solid tumor. In a some embodiments of the invention, the cancer is selected from a group consisting of glioblastoma, nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer, and tonsil cancer.

### (e) Analysis

The biological sample is then analyzed using SILK-SIMS to analyze the masses of charged molecules (ions) in the sample or generated or released by the biological sample. In some embodiments, the biological sample is fixed and processed for SILK-SIMS analysis as previously described in Wildburger, N.C., et al. Amyloid-beta Plaques in Clinical Alzheimer's Disease Brain Incorporate Stable Isotope Tracer In Vivo and Exhibit Nanoscale Heterogeneity. Front Neurol 9, 169 (2018), herein incorporated by reference in its entirety. For example, a focused Cs+ primary ion beam is electrostatically rastered across a defined region-of-interest (ROI) in the biological sample producing secondary ions that are used to measure the atomic composition of the sample surface. SILK-SIMS produces high-resolution quantitative image representing an in situ isotopic-histological map at 100 nm lateral resolution. The acquisition of up to five isotopes in parallel allows detection and localization of a stable isotope tracer within a given ROI by enabling the quantitative image ratio of two stable isotopes of the same element. The incorporation of the tracer ¹³C₆-leucine for instance is detectable by an increase in the ¹³C/¹²C ratio above natural abundance (1.11%) with high sensitivity (0.1-0.2%) and precision.

Various methods and techniques may be employed to calculate molecular flux rates from the nanoSIMS data generated. For example, molecular flux rates may be calculated based on the content, rate of incorporation and/or pattern or rate of change in content and/or pattern of isotope labeling of the molecules of interest. In one embodiment, metabolic flux can be calculated by combinatorial probability and other mass isotopomer analytic methods known in the art. Typical kinetic parameters include, for example, synthesis rates, degradation rates, turnover rates, transport dynamics, metabolic sources, anatomic origins, subcellular interactions, oxidation, reduction, polymerization, conjugation, cleavage, addition, re-arrangement, transport, storage, secretion, or uptake; or the metabolic source or precursor pool used for biosynthesis; or other metabolic processes for each molecule or set of molecules.

Identification of the biosynthetic rate of a molecule is ultimately dictated by an enrichment or depletion in one or more mass isotopologues associated with that molecule. This general principle is extended to algorithms that model the isotopic pattern to best represent the detected signal. This process is applied throughout the data to identify spatially-defined biosynthetic rates. Methods and algorithms are known and described by Hellerstein M K, Christiansen M, Kaempfer S, Kletke C, Wu K, Reid J S, Mulligan K, Hellerstein N S, Shackleton C H, “Measurement of de novo hepatic lipogenesis in humans using stable isotopes,” J Clin Invest. 1991 May; 87(5):1841-52; Hellerstein M K, Neese R A, “Mass isotopomer distribution analysis: a technique for measuring biosynthesis and turnover of polymers,” Am J Physiol. 1992 November; 263(5 Pt 1):E988-1001; Sperling E, Bunner A E, Sykes M T, Williamson J R, “Quantitative analysis of isotope distributions in proteomic mass spectrometry using least-squares Fourier transform convolution,” Anal Chem. 2008 Jul. 1; 80(13):4906-17. Epub 2008 Jun. 4; Rockwood A L, Kushnir M M, Nelson G J., “Dissociation of individual isotopic peaks: predicting isotopic distributions of product ions in MSn,” J Am Soc Mass Spectrom. 2003 April; 14(4):311-22, and in all of which are hereby incorporated by reference in their entireties. Conversion of the resulting mass spectrometry data into metabolic flux data corresponding to spatially-defined locations of the sample can be accomplished by a computer processor with software that processes the relative abundance of mass isotopomers across the spatially-defined locations of the sample.

In some embodiments, the resulting SILK-SIMS data may be converted into metabolic flux images. Each pixel of an image is an elemental unit that represents metabolic flux data. Each pixel is also addressable to a spatial location of the sample, with a known spatial relationship to the other pixels in the image. The spatial location of a pixel in the image corresponds to the metabolic flux rate data of volatile molecules in a corresponding spatial location of the sample. In one embodiment, the metabolic flux image displayed may be the same size as the actual sample. In other embodiments, the metabolic flux image displayed may be smaller or larger than the actual sample. The image may be two-dimensional or three-dimensional. Analysis of serial sections from a sample allows assembly of three-dimensional metabolic flux images of a tissue.

In one embodiment of flux imaging, the relative abundances of mass isotopomers or the pattern of mass isotopomer abundances detected can be characterized down to a pixel-by-pixel basis across the spatial locations of the sample. In one embodiment, mass isotopomers are quantified for individual but more often a plurality of ion envelopes representing biomolecules of interest and analyzed by mathematical algorithms and software programs that are described herein, for instance described in the examples. The pixel-by-pixel changes in mass isotopomer abundance patterns induced by the preceding in vivo metabolic labeling protocol reveals information about the spatially-localized kinetics or metabolic flux of each biomolecule detected as an ion envelope. For example, one to dozens, hundreds or thousands of volatilized molecules can be monitored as a metabolic flux fingerprint or signature of a tissue sample, a specific area of the tissue, or to localize a flux signature to a specific area of the tissue.

The image of each molecule's kinetics can be displayed as a heat map or a topologic map of the sample or other visualization techniques common to the art. In some embodiments, groups of molecules having similar kinetics across spatial coordinates are collapsed into a single representative image. In some embodiments, the mass analyzer can monitor one to thousands of molecules for each pixel, and each molecule monitored can be mapped and displayed as a separate image. The patterns or ratios of a plurality of molecules can also be mapped and displayed.

In one embodiment, overlaying images of the same section of a tissue preparation or from adjacent serial sections of the same tissue preparation, using other histopathologic methods known in the art, such as vital dyes, in situ hybridization, or immunohistochemistry, to correlate metabolic fluxes and functional processes based on their shared spatial coordinates with specific cell types, subcellular organelles, molecular aggregates or other known morphologic features of a tissue.

In other embodiments, the output of metabolic flux data corresponding to spatially-defined locations of the sample with known spatial relationships may be in the form of a table or a database.

The output generated according to the methods described herein represents kinetic data corresponding to known spatial coordinates of the sample analyzed. The methods and software described herein permit the visual representation of data as functional metabolic processes, in the form of heat maps, contour maps or other images by spatial coordinates in a biologic tissue or cell preparation. By way of non-limiting examples, said images may include, for example, plaque dynamics of neuropathies in the brain; glucose uptake by cancer cells; the spatial topology of mitochondrial lipid synthesis in muscle cells; of the spatial distribution of prostanoid and eicosanoid turnover in inflammatory infiltrate tissues; of the pattern of lipogenesis in biopsies of cancer or precancer, and the presence of functional hot spots within a tumor; of the topology of hormonal synthesis in an endocrine tissue; for the presence of autonomous functional areas; for localization of regenerating cells and cell membranes, in a tissue, as in peripheral neuropathies; for the identification of spatially-localized timed biosynthetic events in a tissue based on calculated precursor pool enrichments; and many other means of representing the dense information generated about metabolic fluxes in space and time.

In some embodiments, the methods and software can make use of univariate and multivariate statistical algorithms such as the analysis of variance, k-means clustering, principle component analysis, non-negative matrix factorization, and other approaches known to the field to grouping patterns of similar molecular distribution patterns and flux distributions patterns.

In some embodiments, the methods can use at least two different biomolecules to resolve and identify adducts, degradation products, and multiple charge states for molecules. Molecules which can be monitored include molecules such as sugars, polysaccharides, lipids, metabolites, proteins, enzymes, nucleotides, etc. In some embodiments the two different biomolecules result in the concurrence of the at least two labels on a single biomolecule or chemical structure.

Given that control over biological processes is generally exerted as rate control, by the regulation of catalytic reactions and the partitioning of molecules through competing pathways, metabolic fluxes directly reveal the activity of functional processes and pathways in a tissue and often have functional significance in their own right. Because of the information density and spatial definition of the metabolic flux data produced by the method disclosed herein, biologically or medically heterogeneities useful information can be gleaned from metabolic flux patterns that are observed within a tissue, such as regions of increased or reduced metabolic fluxes, regions that differ or are similar for metabolic fluxes, complex signatures of metabolic fluxes for multiple molecules, complex patterns or gradients of metabolic fluxes for specific cells, organelles or structures, or other quantitative parameters related to the metabolic fluxes detected. Specifically, unique functional information about a tissue can be inferred from spatially-identified and patterns of dynamic processes (e.g., the degree of heterogeneity; the ratios of different molecular flux rates in selected areas of the tissue; regions of the tissue that are metabolically linked; shared or differing metabolic precursor pools; etc.), providing potential signatures of each individual's disease phenotype that have prognostic or therapeutic significance. Spatially-identified heterogeneities and patterns of dynamic processes can also focus more in-depth further analysis to specific regions of the tissue or to molecules or metabolic pathways that are identified as being altered and of interest.

The combination of SILK-SIMS can reveal cell-specific or subcellular structure-specific functional information throughout a tissue, without the need of traditional static hispathological markers. Interrogation of tissue specimens collected from subjects with conditions such as cancer, inflammation, neurologic disorders, immune diseases, infections, fibrotic diseases, diabetes, obesity, arteriosclerosis, endocrine disorders, etc. for functional metabolic flux mapping and metabolic flux signatures thereby provides a novel and powerful tool for characterizing the phenotype (behavior, prognosis, pathogenic sub-class, optimal treatment strategy, response to ongoing treatment, etc.) for a tissue or disease.

SILK-SIMS provides numerous applications in medical or veterinary diagnostics, companion diagnostics, drug discovery, drug efficacy and development and biologic research are evident, and are described herein. These include functional histopathologic display and mapping in disease tissues such as cancer, fibrosis, inflammation, metabolic disorders, atherosclerosis or neuropathology, for diagnosis, therapeutic targeting, patient stratification and personalized medicine. In particular, kinetic signatures or fingerprints in a tissue can be correlated with disease behavior or treatment response, for use in medical or veterinary disease management or in medical diagnosis and companion diagnostics.

Biomedical applications of SILK-SIMS, include for example functional imaging of histopathology in disease tissues, such as cancer, fibrosis, inflammation, metabolic disorders, neuropathology, for spatial in homogeneities that reveal areas of increased or reduced rates of a functional process (hot spots or cold spots, respectively), for diagnosis, therapeutic targeting, patient stratification or personalized medicine. Specific applications, for example, mapping cholesterol turnover in the core of an atherosclerotic plaque in a blood vessel, and the capacity of a high-density lipoprotein treatment to mobilize cholesterol from the core of a plaque; imaging autophagic pathways fluxes based on the turnover of proteins or peptides derived from proteins that are autophagic substrates, in a different regions of a cancerous tissue, neurologic tissue, or muscle tissue; displaying lipid synthetic fluxes or structural protein synthetic fluxes in different cellular compartments of muscle tissue from a sarcopenic or cachectic subject, including cardiolipin turnover in mitochodria, fatty acid synthesis and turnover in myocytes and in the extracellular space, as a biomarker of muscle quality or response to treatment; measuring the turnover of aggregated proteins, such as amyloid beta in Alzheimer's plaque, huntingtin or alpha-synuclein in neurodegenerative diseases, or of cellular storage granules, such as insulin in pancreatic beta cells; monitoring loss of labeled palmitate, glucose or other energy substrates from oxidative tissues like skeletal muscle or failing heart, as a marker of fuel utilization by specific cells in a tissue; visualizing myelin synthesis in the central or peripheral nervous system, in settings of demyelination, neurodegeneration or neuropathy; displaying the turnover of cell membrane receptors in disease states such as the epithelial sodium transporter in hypertension or the CFTR in bronchi in cystic fibrosis, LDL cholesterol receptor turnover in tissues from hyperlipidemic subjects and in response to lipid-lowering agents; metabolic conversion of steroid hormones to their active forms and target sites in a target organ, such as testosterone reduction to dihydrotestosterone in prostate tissue or muscle specimens and the effect of dihydrotestosterone inhibitors.

SILK-SIMS may also be used for functional imaging of disease tissues, such as cancer, for kinetic signatures correlated with disease behavior or treatment response, for use in medical or veterinary disease management or in medical diagnosis and companion diagnostics. Specific applications include, for example, mapping lipid metabolic fluxes and protein turnover across cancer tissue slides, to identify hot spots and heterogeneity, as a marker of cancer aggressiveness or response to treatment; measuring lipid flux patterns in tissues potentially exhibiting lipotoxicity, such as muscle, pancreas and liver, to identify metabolic flux fingerprints associated with insulin resistance or diabetes risk; imaging patterns of lipid turnover in areas of skin in subjects with eczema or psoriasis as signatures of disease behavior or likely response to treatments, including response to cosmetic treatments; monitoring the patterns of transport of cargo proteins along neurons in different areas on the brain in neurodegenerative diseases; and many others apparent in the art.

SILK-SIMS may also be used for determination of the timing of spatially-localized kinetic processes, such as embryologic or other developmental events, by imposition of timed precursor label administration or a temporal gradient of precursor label administration, and displaying precursor pool enrichments for molecules in different locations within a tissue; determination of biosynthetic origins or metabolic sources of molecules in spatially-localized regions of cells (e.g., identifying the tissue or subcellular origin of transported molecules); or characterization of subcellular functional organization—for example, kinetic processes in subcellular organelles, lipid droplets, storage granules, secretory vesicles, endoplasmic reticulum, etc. as a tool for understanding the in vivo regulation and control of metabolic flux in a tissue.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Silk-SIMS of Human Alzheimer's Disease Plaques

The following example demonstrates in vivo stable isotope labeling & quantitative mass spectrometry imaging of Aβ plaque deposition in human AD brain and the localization and quantification of the stable, non-radioactive isotope ¹³C in cells, mouse brains, and human brain.

In vivo stable isotope labeling & quantitative mass spectrometry imaging of Aβ plaque deposition in human AD brain Alzheimer's disease (AD) is characterized by alterations in the clearance of amyloid-β (Aβ) in the brain^{1,2,3,4}. Studies utilizing in vivo incorporation of the stable isotope ¹³C₆-leucine into Aβ in AD patients^{1,2} demonstrated that while the production of Aβ40 and 42 are similar between AD and control (non-AD, age-matched) subjects, the clearance of Aβ is decreased ˜30-50%^{3,4}. At the onset of amyloidosis, the Aβ42 proteoform demonstrates faster turnover kinetics, possibly due to rapid deposition into plaques (˜50% of all Aβ42 produced) as suggested by a positive correlation between increased Aβ42 turnover kinetics and rate of fibrillar amyloidosis measured by PET Pittsburgh compound B (PIB)⁴. However, these studies⁴ relied on CSF, which are indirect kinetic measurements of brain compartment Aβ. Recent evidence also demonstrates that PIB binds to only a subset of Aβ from plaques enriched from AD brain⁵. The implication of these recent findings suggests that studies utilizing PIB may be potentially underestimating the extent and rate of Aβ deposition and/or only measuring localized fibrillar pathology. The examples provided herein report the first measurements of protein deposition into amyloid-β plaques in human AD brain and an APP/PS1 mouse model of AD. Post-mortem brain from participants labeled with ¹³C₆-leucine in vivo for stable isotope labeling kinetics (SILK) combined with nanoscale Secondary Ion Mass Spectroscopy (NanoSIMS) imaging was used in a combined approach termed SILK-SIMS to quantity Aβ plaque deposition in human AD brain and APP/PS1 mice. The inventors found in controlled imaging experiments an isotopic enrichment of the ¹³C isotope above the natural abundance ratio of ¹³C/¹²C (1.1%) restricted to the periphery of Aβ plaques. In APP/PS1 mice orally labeled for 10 weeks starting before plaque pathology, the ¹³C/¹²C isotope ratio was 2.7% with distinctly higher enrichment at the periphery compared to the core of the plaque. Metabolically active cells such as a nearby neuron demonstrated ¹³C/¹²C ratio of 3.7%. These results demonstrate that amyloid plaque deposition can be quantified to address the unanswered question of “What is the rate of amyloid Alzheimer's pathology in humans?” It is anticipated that these results will lead to a more accurate estimate of plaque growth, which can be utilized to determine the rate of Aβ pathology prior to the onset of AD clinical symptoms. New insights into amyloid kinetics in vivo will inform efforts for improved diagnostic test(s) to detect the early, prodromal stage of AD, accelerate AD drug trial development, and facilitate a direct comparison between depositions determined by SILK-SIMS to those derived from PET-PIB. Furthermore, the protocols established herein can be translated to other disorders including in non-limiting examples neurodegenerative disease, metabolic diseases, and cancer.

The first quantitative measurement of deposition into amyloid plaques and the unique, first-in-human, opportunity to measure Aβ kinetics directly in the brain and AD pathology is described here.

The accumulation of amyloid-beta (Aβ) leads to one of the pathological hallmarks of Alzheimer's disease (AD)—amyloid plaques.⁶ It was first recognized through early biopsy and autopsy studies that the extent of histologically apparent Aβ deposition does not correlate with disease severity⁷⁻¹⁰ or duration.¹¹ In fact, many elderly individuals have extensive Aβ deposition without clinical signs of dementia.^{8,12-15} The development of amyloid imaging agents furthered the notion that plaque pathology, measured by PET-PiB, stabilizes while dementia severity progresses¹⁶⁻¹⁹ in agreement with previous studies, that there is no relationship between Aβ deposition and disease severity or progression.

In recent studies utilizing in vivo incorporation of the stable isotope ¹³C₆-leucine in AD patients,¹⁻⁴ Aβ42 demonstrated faster turnover kinetics. This was attributed to rapid deposition into plaques for ˜50% of all Aβ42 produced⁴ due to a positive correlation between increased Aβ42 turnover kinetics and rate of amyloidosis measured by PET-PiB.⁴ However, accurate measures of plaque growth and turnover—a reflection of the rate and extent of disease pathology—are marred by analytical challenges. In vivo Aβ kinetics⁴ has relied on CSF, which is an indirect measure of the brain compartment. Further, evidence suggests that PiB only binds a subset of Aβ⁵ and repeated studies of amyloid PET may measure binding site changes over time,¹⁶ not necessarily the growth or turnover kinetics of Aβ in human AD brain. Methodological challenges with anti-Aβ antibody or dye specificity and decreased sensitivity due to tissue autofluorescence limit fluorescence-based assessments.

The present studies used stable isotope kinetic labeling (SILK) coupled to nanoscale secondary ion mass spectrometry (NanoSIMS) in a method termed SILK-SIMS to determine whether plaques are dynamic structures with growth and turnover rates that can be directly measured in human AD brain. In SILK-SIMS, a focused Cs⁺ primary ion beam is electrostatically rastered across a defined region-of-interest (ROI) in the tissue producing secondary ions that are used to measure the atomic composition of the sample surface. SILK-SIMS produces high-resolution quantitative image representing an in situ isotopic-histological map at 100 nm lateral resolution. The acquisition of up to five isotopes in parallel allows detection and localization of a stable, non-radioactive isotope tracer within a given ROI by enabling the quantitative image ratio of two stable isotopes of the same element.²⁰ The incorporation of the tracer ¹³C₆-leucine for instance is detectable by an increase in the ¹³C/¹²C ratio above natural abundance (1.11%) with high sensitivity (0.1-0.2%) and precision.

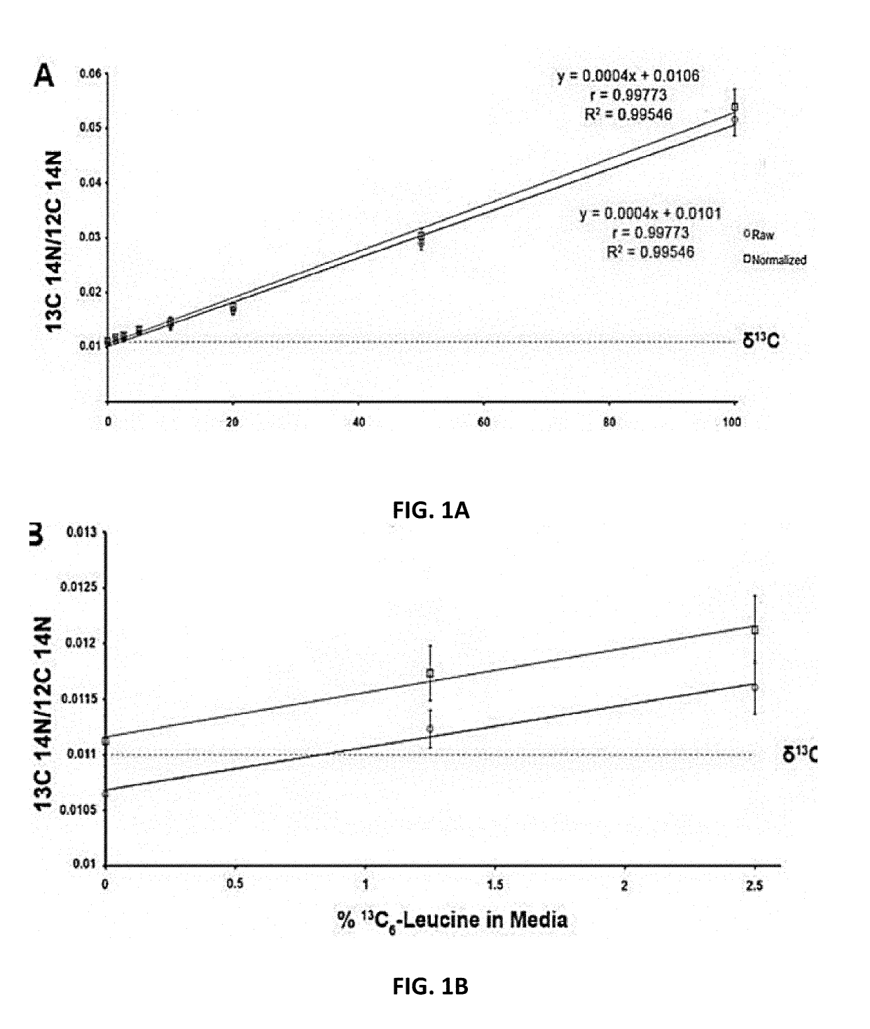

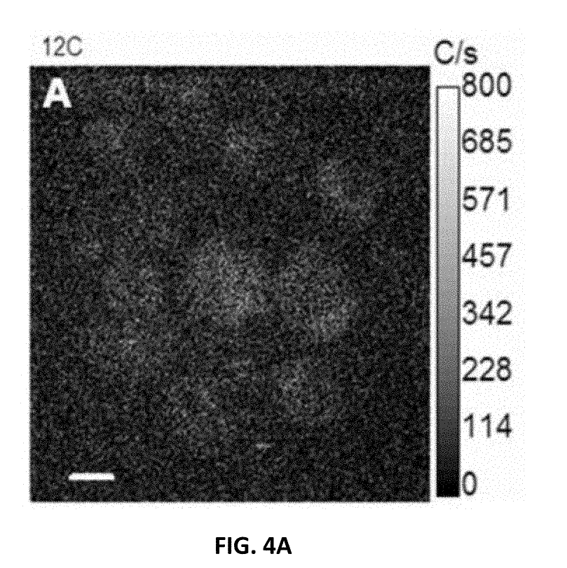

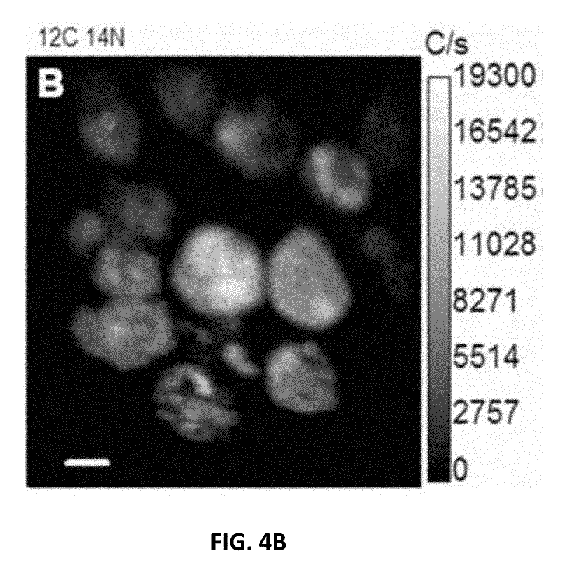

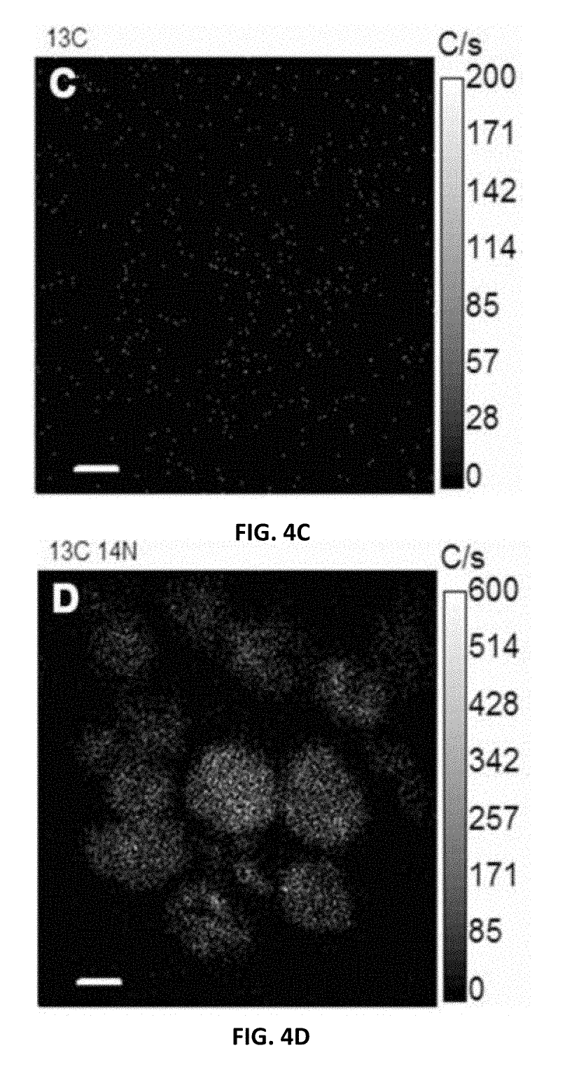

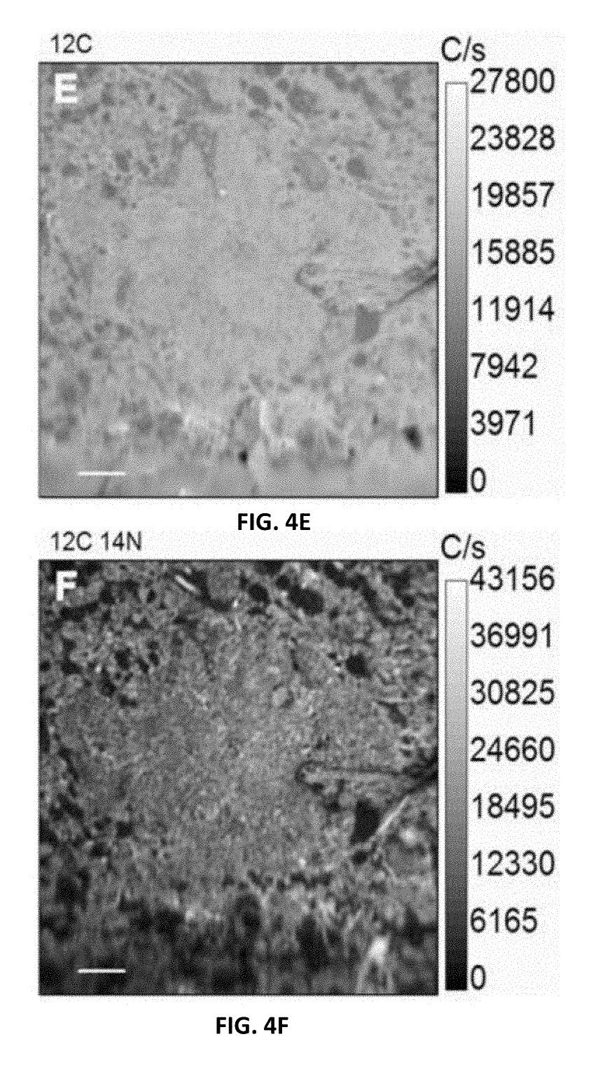

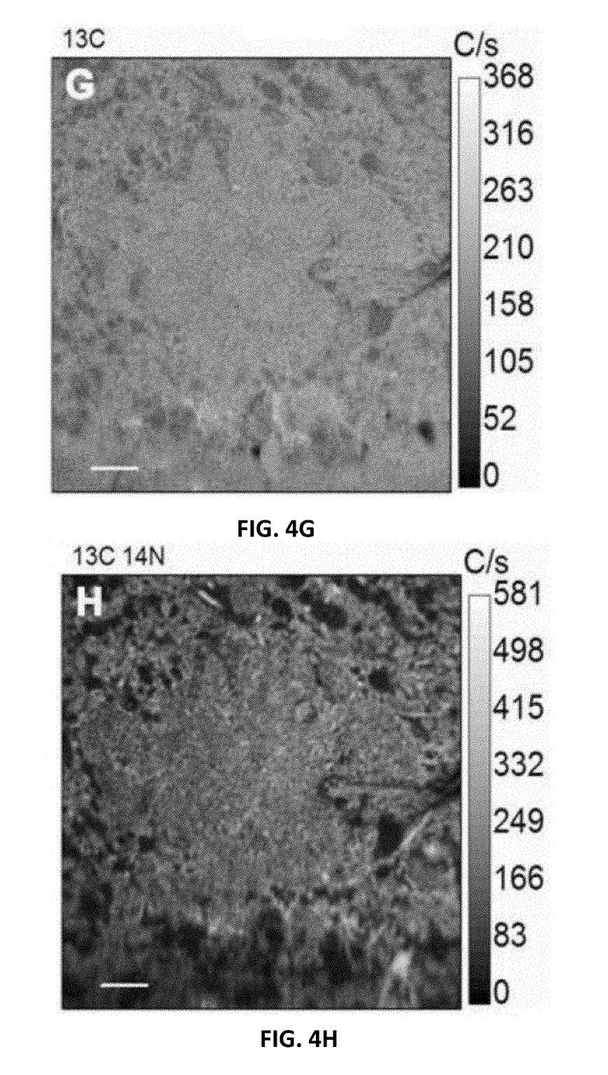

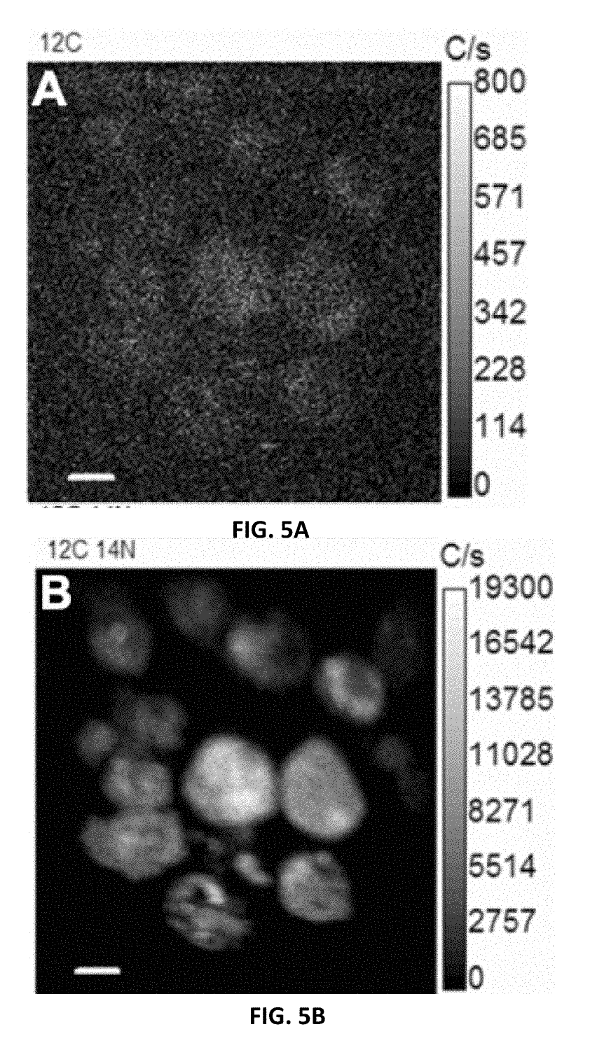

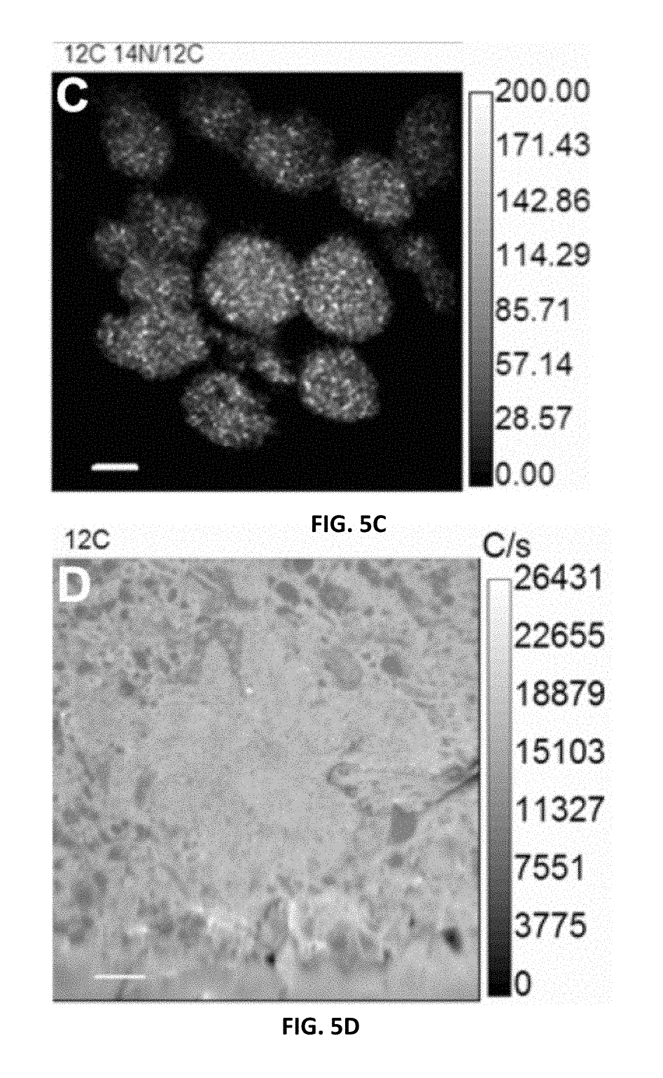

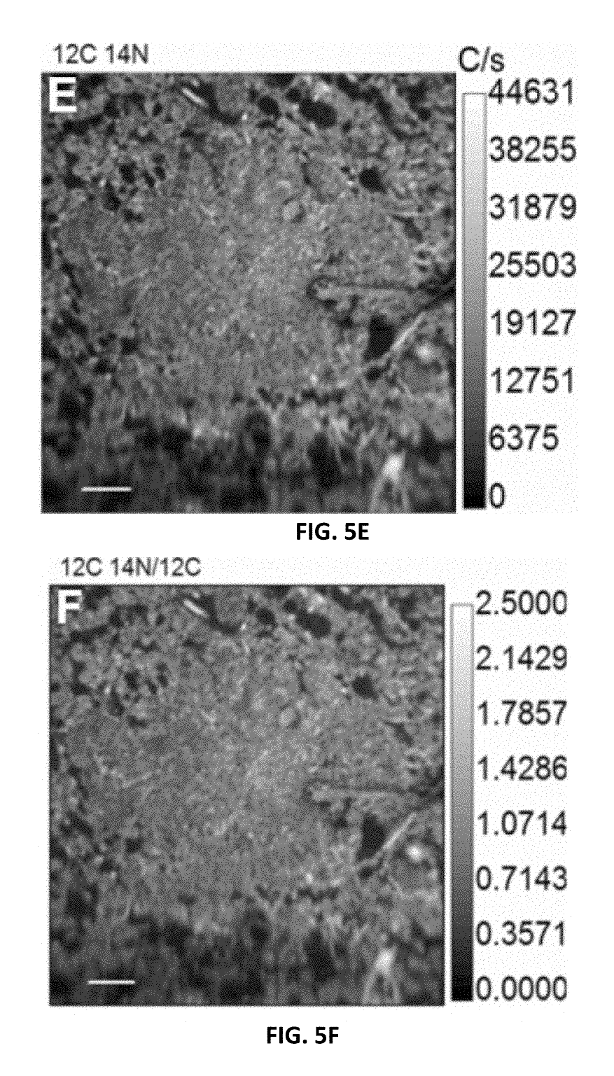

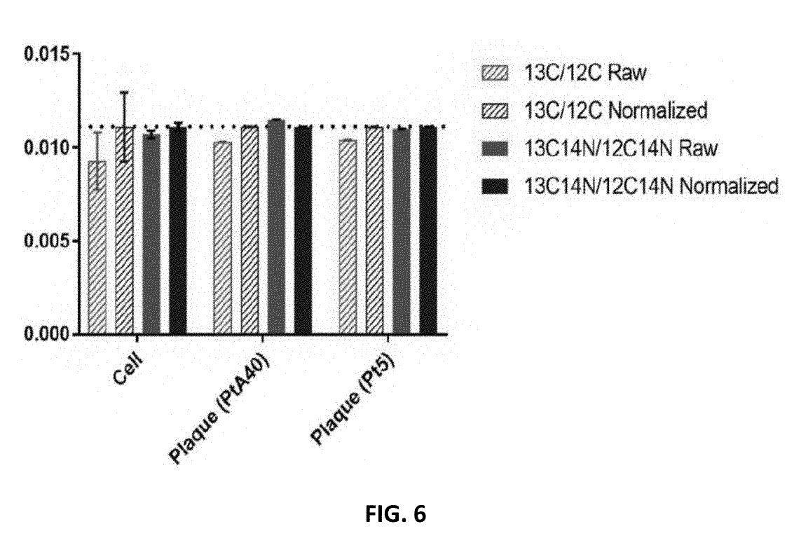

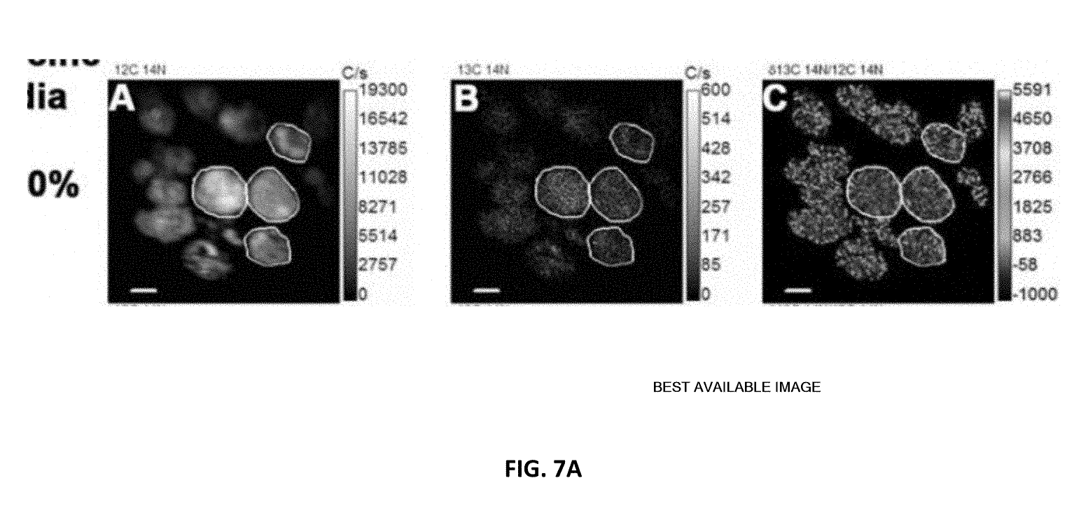

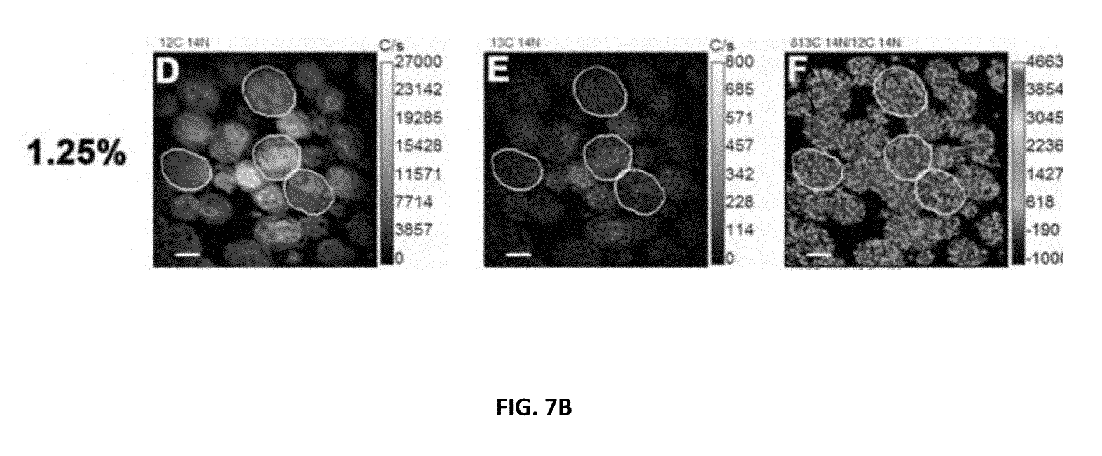

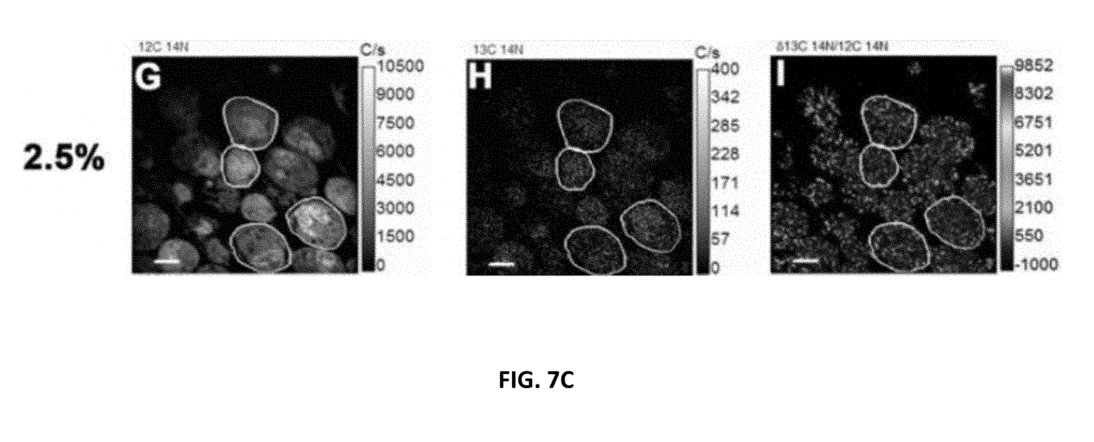

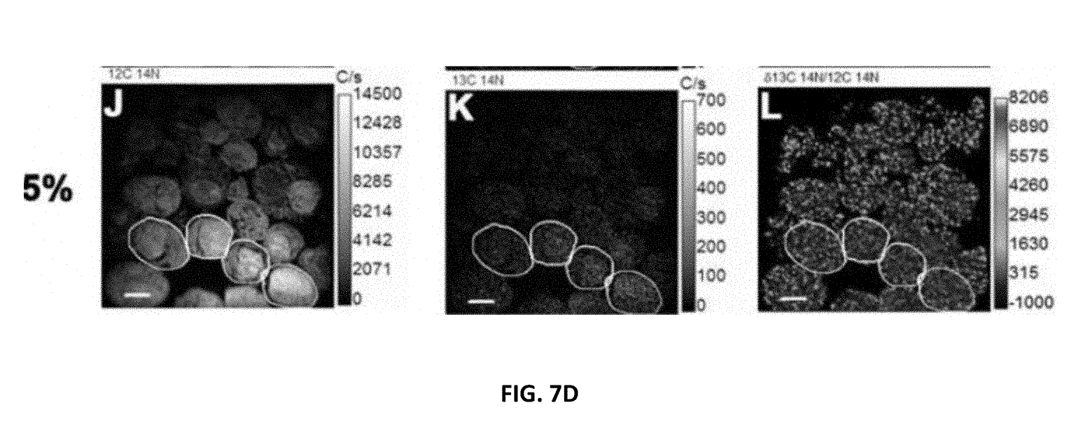

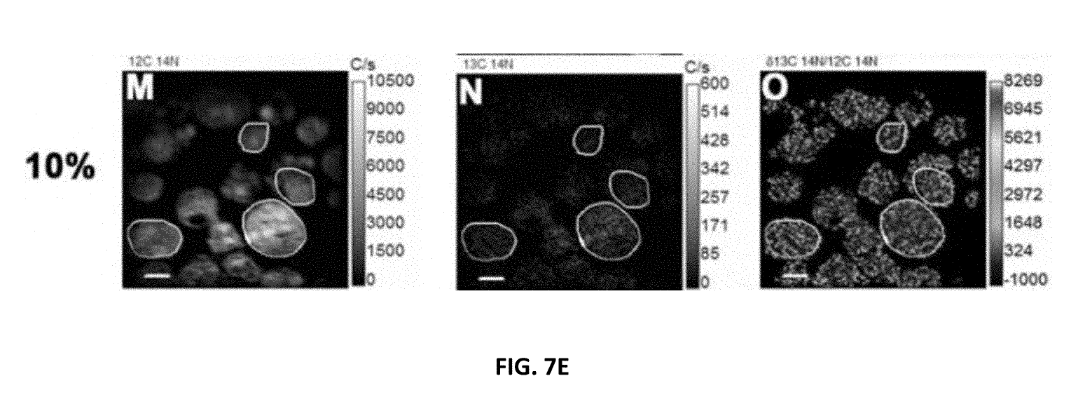

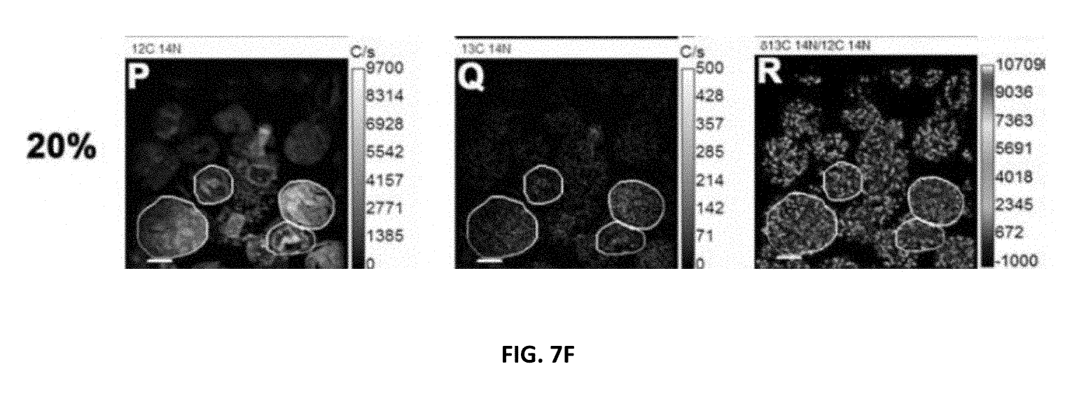

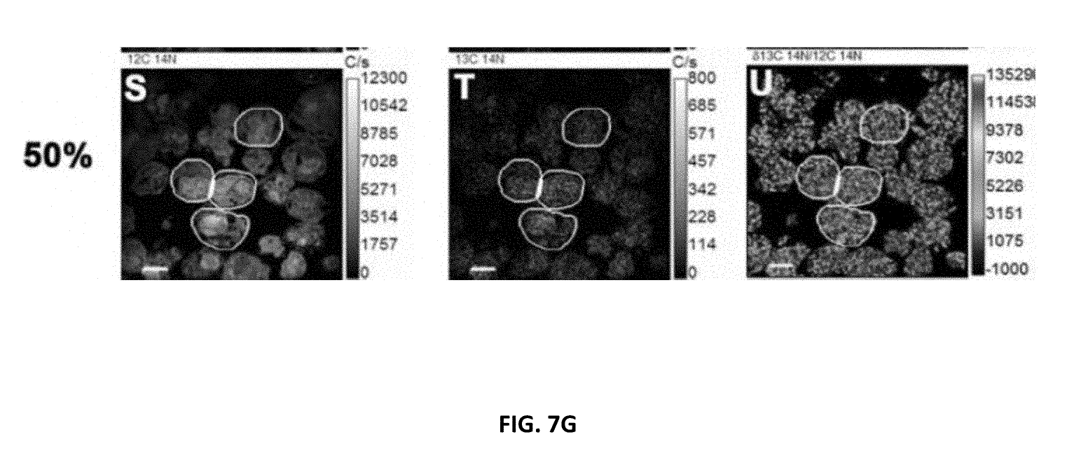

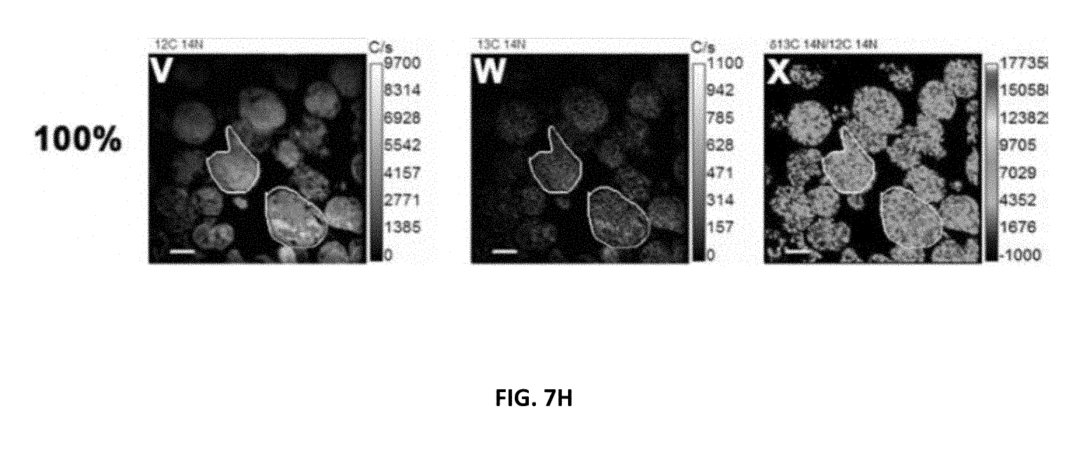

To quantify carbon isotopes in biological material, carbon was measured as ¹²C⁻, ¹³C⁻, ¹²C¹⁴N⁻, and ¹³C¹⁴N⁻ simultaneously. Evident from these measurements is the improved image quality when carbon isotopes were detected as cyanide ions (¹²C¹⁴N⁻ and ¹³C¹⁴N⁻) rather than monoisotopes (FIG. 4). The improved isotope image quality with detailed histology may be due to i) the higher ionization potential of cyanide ions compared to carbon as evidenced by the signal intensity (Cts/s), ii) the nitrogen content of biological materials with CN⁻ molecules being most abundant in proteins (18% by total weight) compared to RNA and DNA (1.1% and 0.25%, respectively)^{20,21} and iii) the reduction of carbon contribution of the embedding media, which contributes a large fraction of ¹²C⁻ ions²⁰ (FIG. 5). Quantitatively, data analysis using the CN⁻ molecules achieves improved accuracy and precision compared to C⁻ alone (FIG. 6). Using CN⁻ we characterized the NanoSIMS signal response for ¹³C₆-leucine enrichment, by labeling a B-cell hybridoma grown in leucine-free media supplemented with an increasing percentage of ¹³C₆-leucine. FIG. 1 demonstrates the linearity of response in NanoSIMS measurements of cells treated with increasing percent of ¹³C₆-leucine (R²=0.99546) and a raw and normalized y-intercept of 0.0106±1.53×10⁻⁴ and 0.0111±2.25×10⁻⁴, respectively. NanoSIMS measurement accuracies are within 3-6% and ±2% of the natural abundance of ¹³C and a precision of ±0% and ±0.05% for raw and normalized data, respectively (FIG. 7).

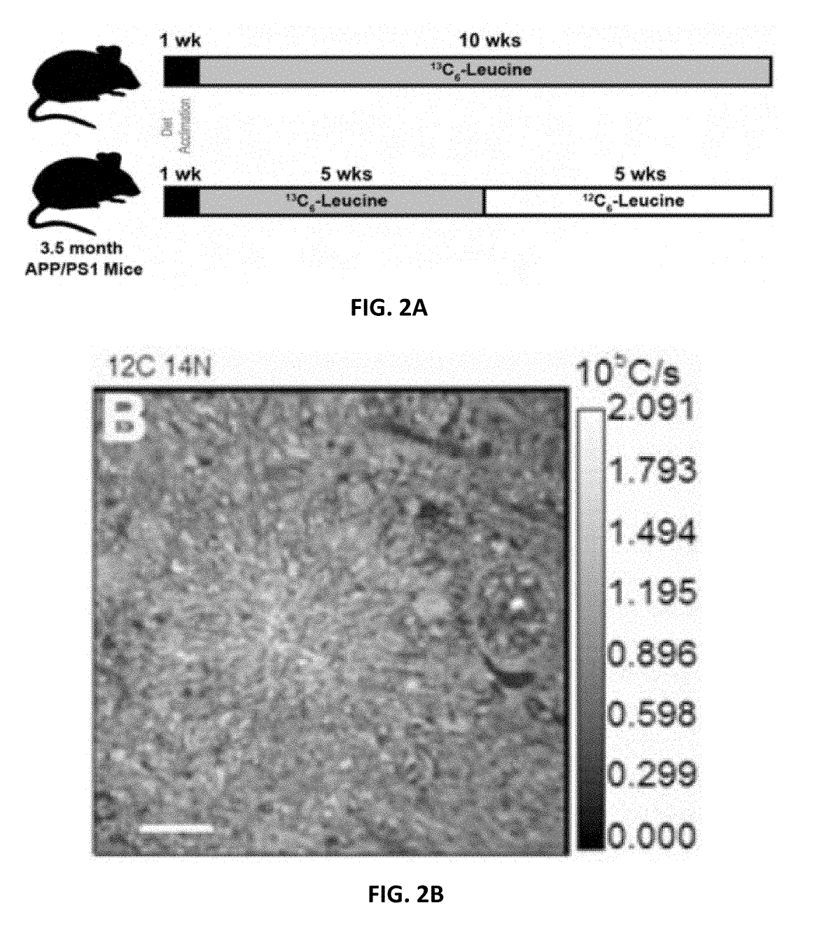

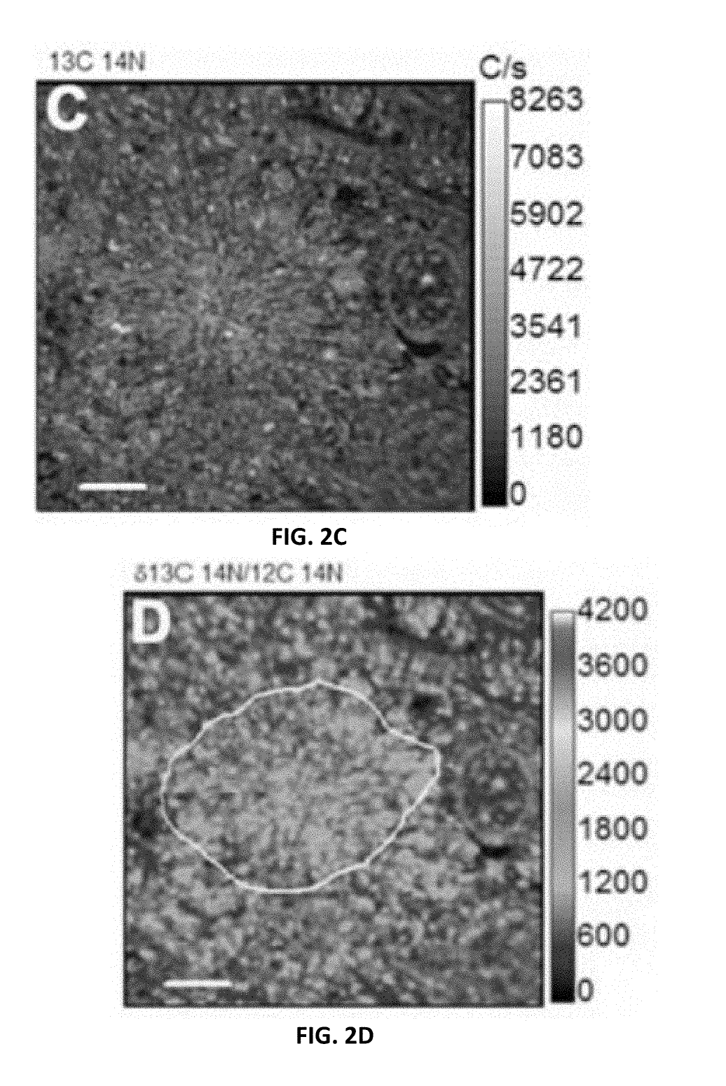

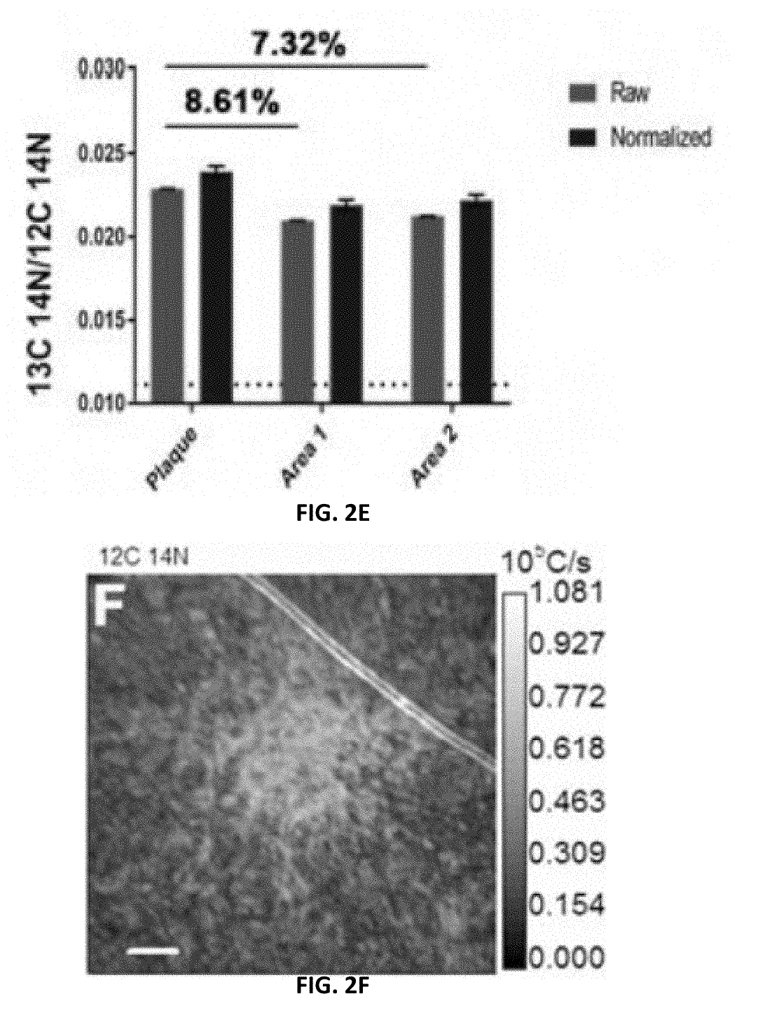

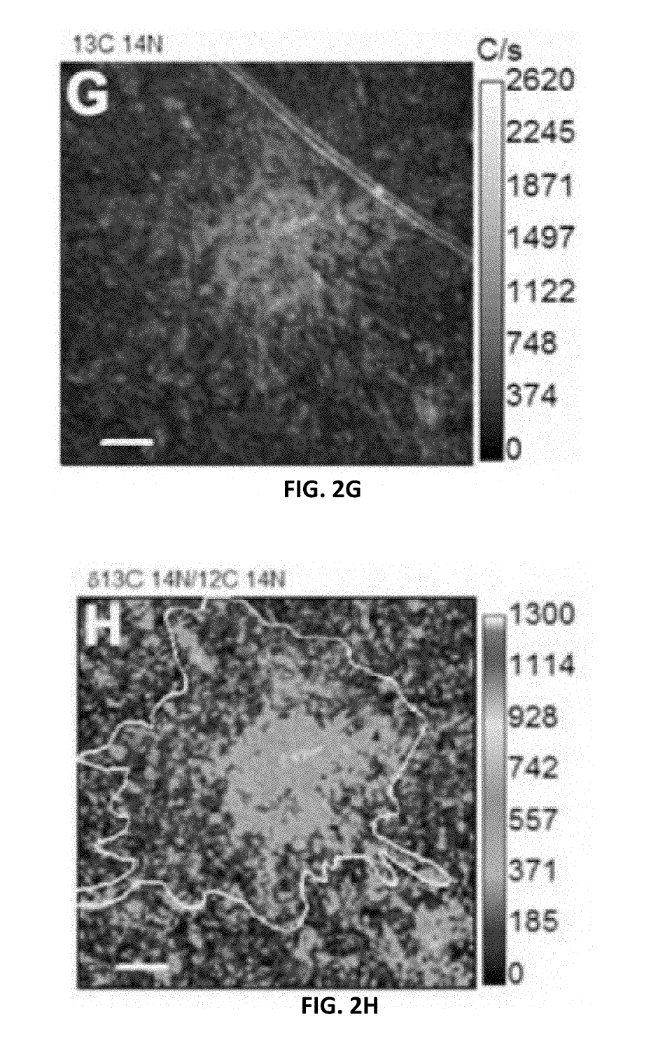

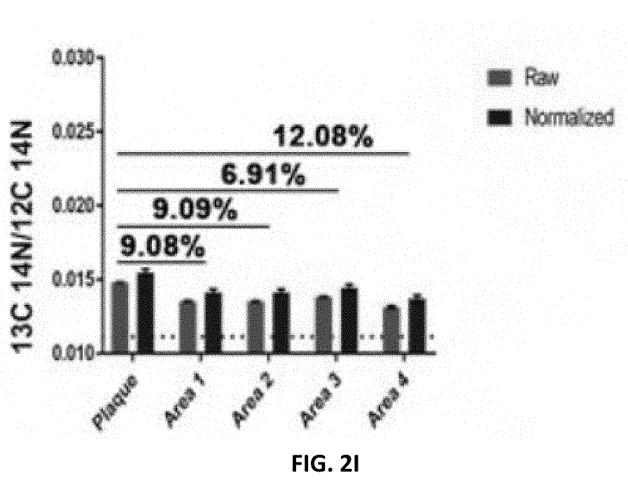

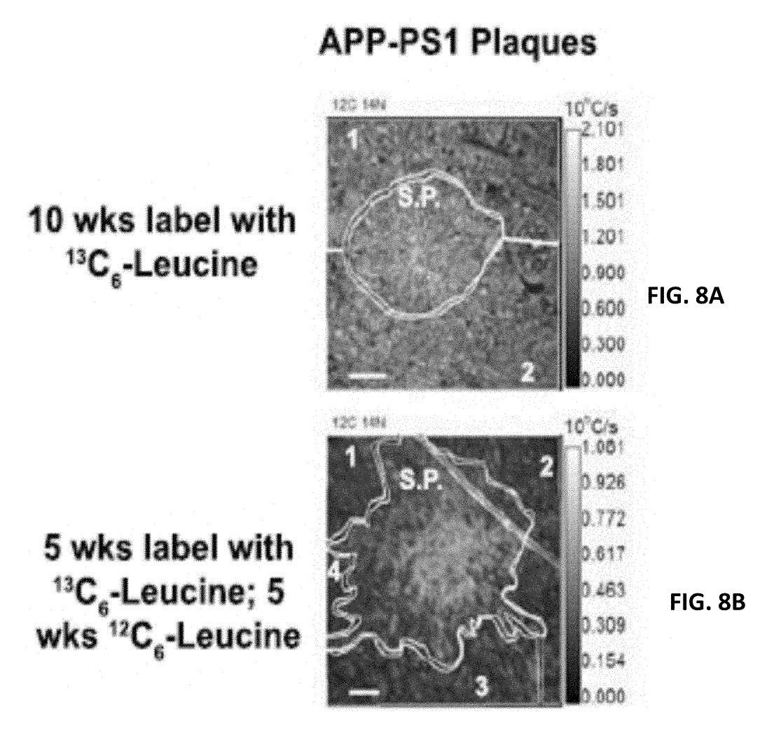

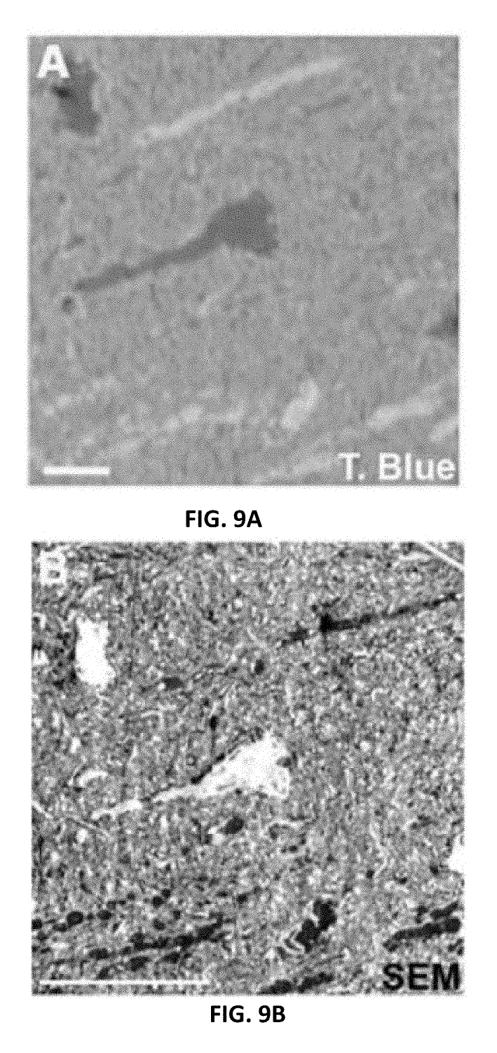

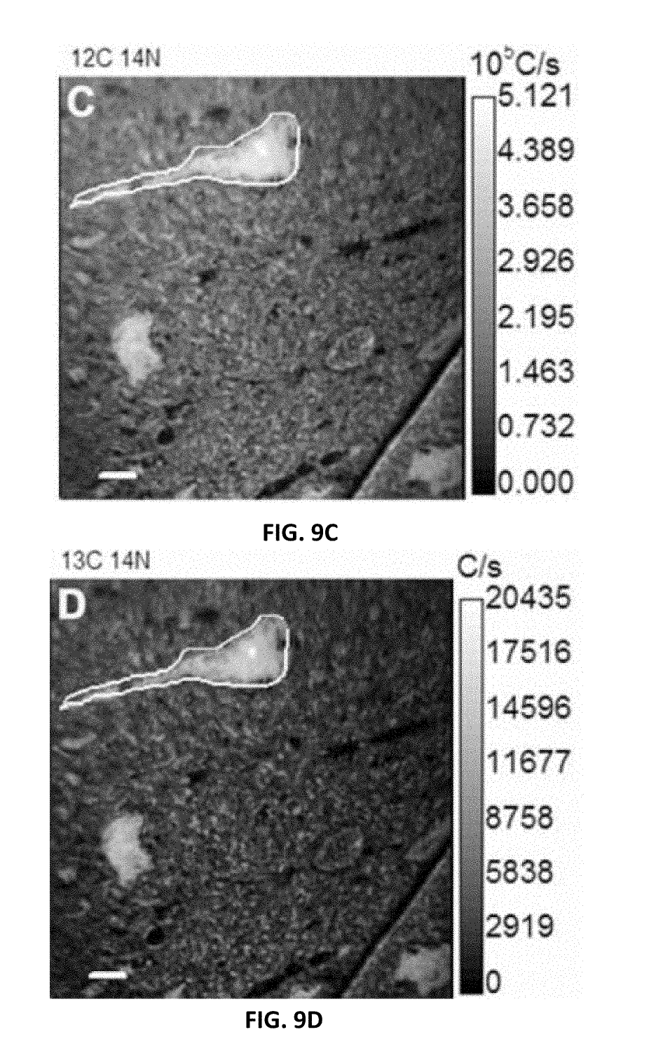

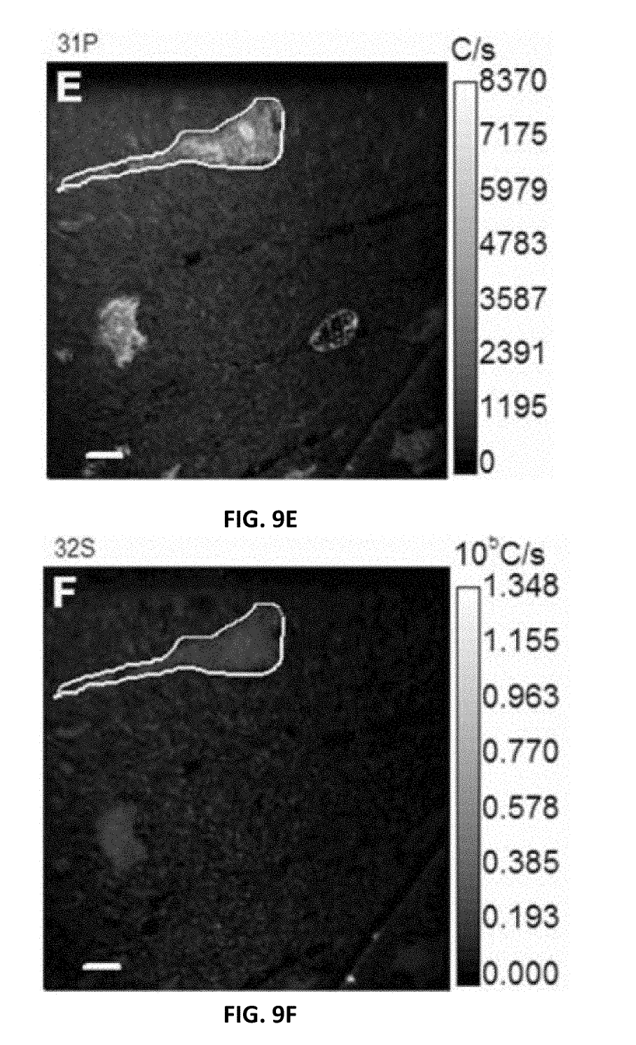

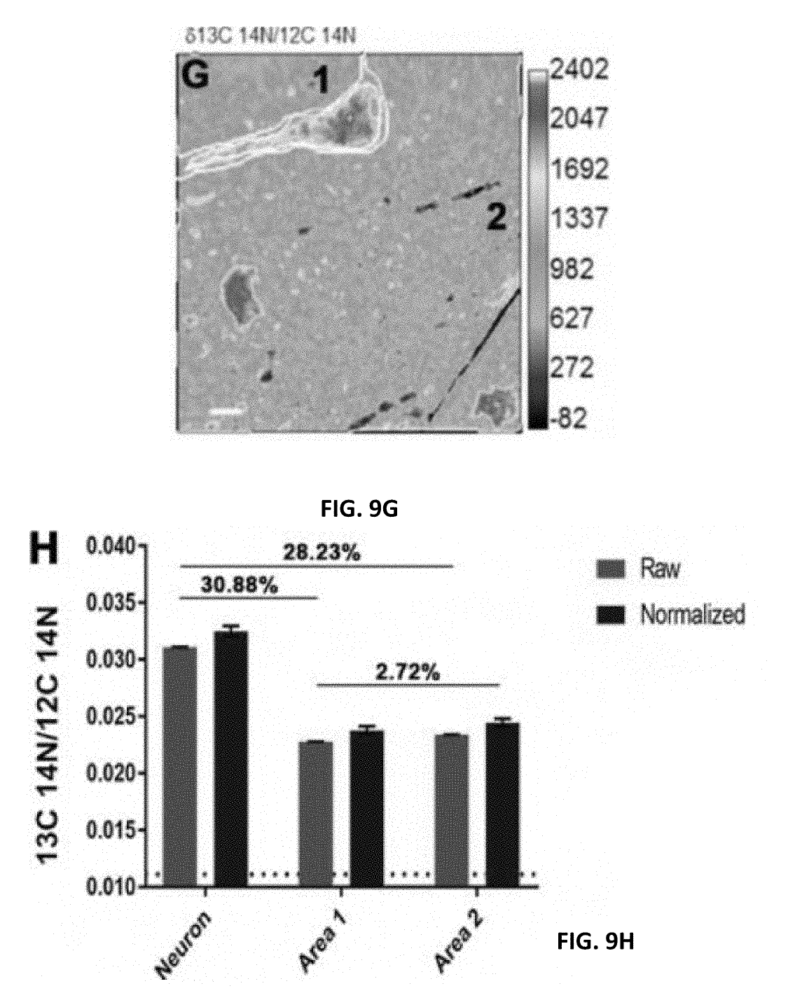

To assess the feasibility of detecting ¹³C₆-leucine isotopic enrichment in native amyloid-β plaques, tracer was administered to APP/PS1 mice at 3.5 months of age (pre-plaque pathology) for 10 and 5 weeks (FIG. 2A). The APP/PS1 mouse labeled for 10 consecutive weeks (FIGS. 2B-2E) reached raw and normalized ¹³C¹⁴N⁻/¹²C¹⁴N⁻ ratios of 0.023±1.41×10⁻⁵ and 0.024±3.43×10⁻⁴ respectively, compared to background (i.e., brain parenchyma) in Area 1 (0.021±1.28×10⁻⁵ and 0.022±3.15×10⁻⁴, raw and normalized) and Area 2 (0.021±1.13×10⁻⁵ and 0.022±3.19×10⁻⁴, raw and normalized) (FIG. 8). Thus demonstrating that the incorporation and measurement of ¹³C₆-leucine into Aβ plaques is feasible, but also non-random (i.e., non-homogeneous distribution of incorporated tracer). Features that were more metabolically active incorporated more tracer compared to brain parenchyma. As shown in FIG. 9, a neuron (a hyper-metabolic cell) from the 10-week labeled mouse incorporated substantially more tracer that the surrounding parenchyma (neuron=0.031±2.53×10⁻⁵ and 0.032±4.66×10⁻⁴ vs Area 1=0.023±1.70×10⁻⁵ and 0.024±3.41×10⁻⁴ vs Area 2=0.023±7.17×10⁻⁶ and 0.024±3.50×10⁻⁴ raw and normalized, respectively) and the plaque (0.023±1.41×10⁻⁵ and 0.024±3.43×10⁻⁴ raw and normalized, respectively).

The APP/PS1 mouse labeled for 5 consecutive weeks followed by a 5-week washout period, continued to demonstrate ¹³C₆-leucine tracer enrichment relative to natural abundance (0.015±1.56×10⁻⁵ and 0.015±2.22×10⁻⁴ raw and normalized) and the surrounding parenchyma (FIG. 2F-I). Despite begin labeled for half the amount of time, the plaque enrichment was approximately two-thirds of that measured in the 10-week labeled animal. However, plasma leucine measurements taken from these mice at time of sacrifice show that the ¹³C₆-leucine tracer declined to less than 1/10^{th} of the tracer content found in the 10-week labeled mouse (TABLE 1). This observation would be expected if Aβ plaques had a slower turnover rate. Of note, the ¹³C enrichment (δ) was substantial in the core region of the plaque (˜700‰ or 70%), but much reduced in the peripheral region (˜190‰ or 19%) around the plaque representing either growth after the 5-week tracer period or washout from the periphery inward of a fully labeled plaque.

TABLE 1
Plasma leucine in labeled APP/PS1 mice

| | ¹²C₆- | ¹³C₆- | | | | Average |
|---|---|---|---|---|---|---|
| | Leucine | Leucine | | | Mol | Mol |
| | Peak | Peak | | | fraction | fraction |
| | Area | Area | ¹³C/¹²C | TTR^{(a)} | labeled | labeled |
| 10-week | 4579 | 11793 | 257.545% | 257.495% | 72.03% | 72.02% |
| 10-week | 4601 | 11845 | 257.444% | 257.394% | 72.02% | |
| 5-week | 14438 | 2878 | 19.934% | 19.884% | 16.59% | 16.30% |
| 5-week | 14256 | 2724 | 19.108% | 19.058% | 16.01% | |

TTR. Tracer-to-Tracee Ratio
^{(a)}Mole farction of labeled leucine

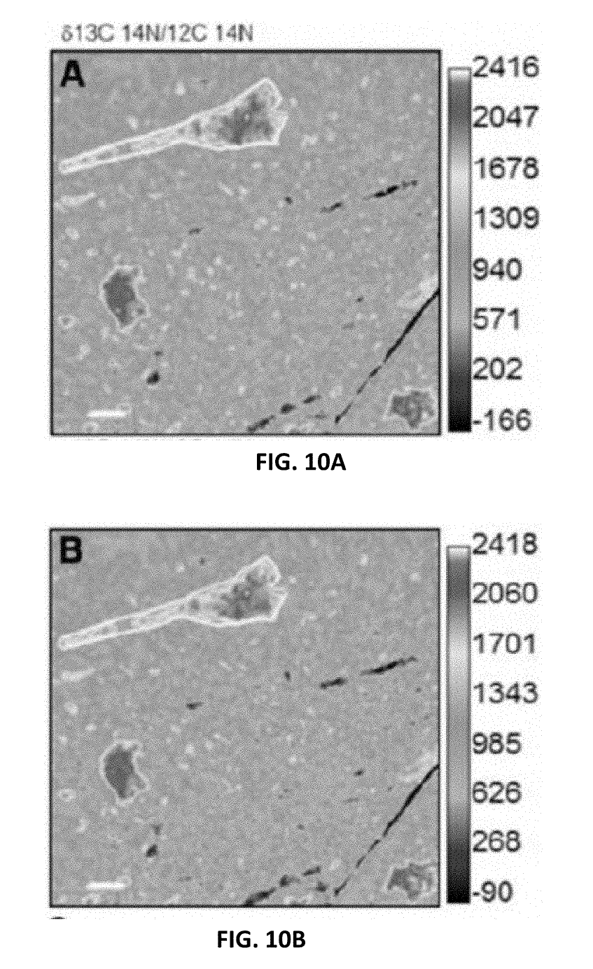

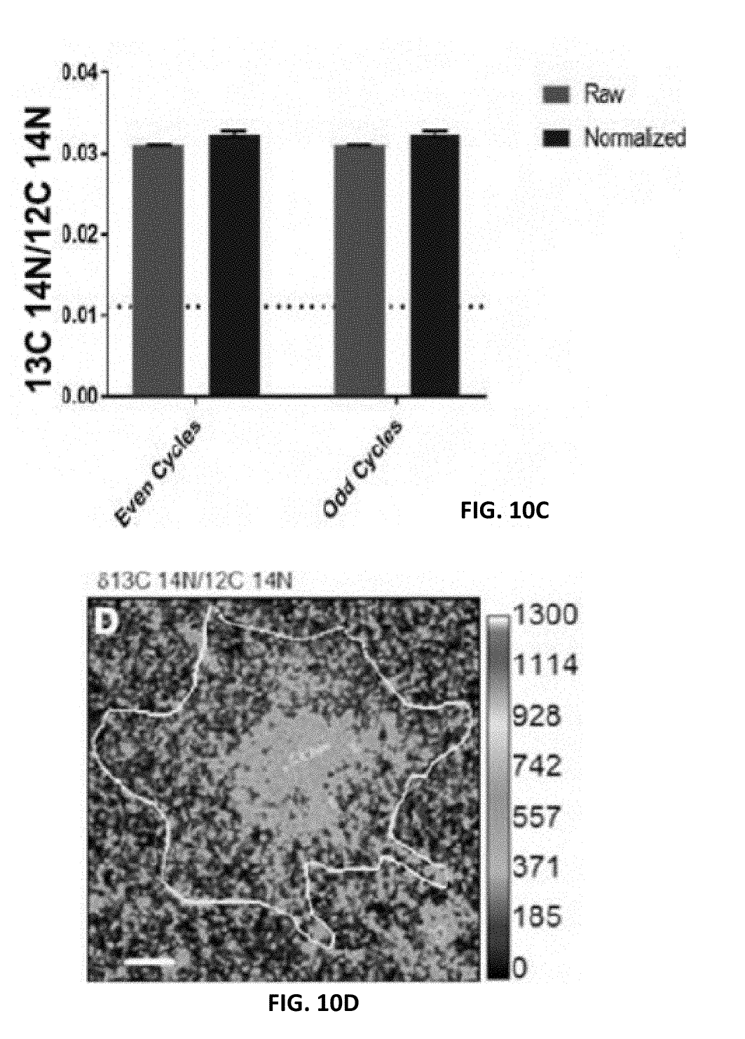

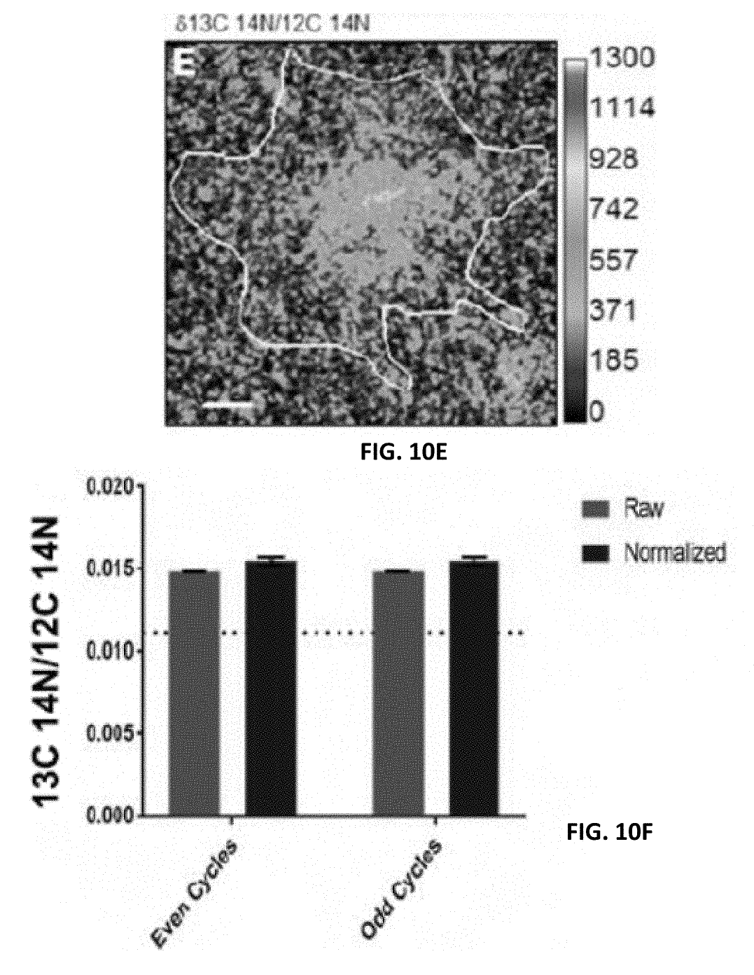

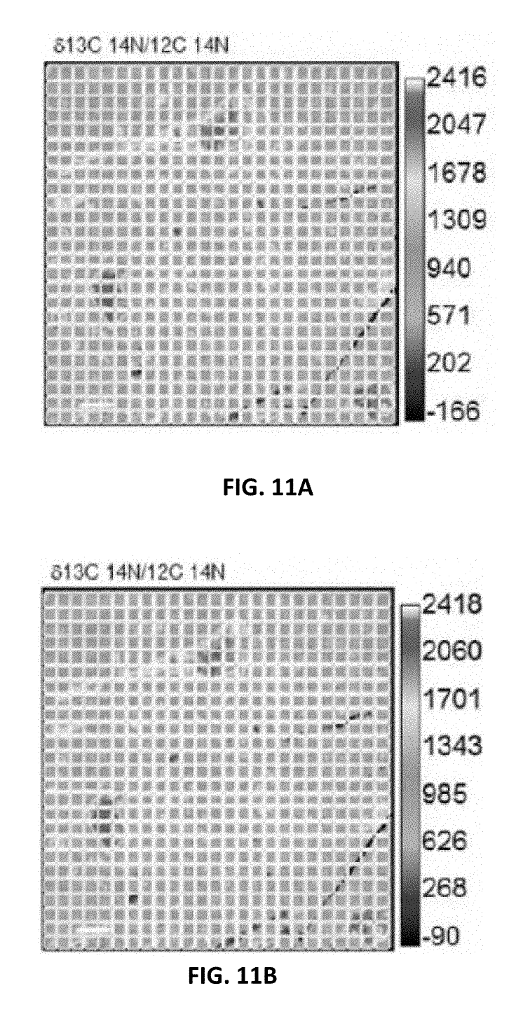

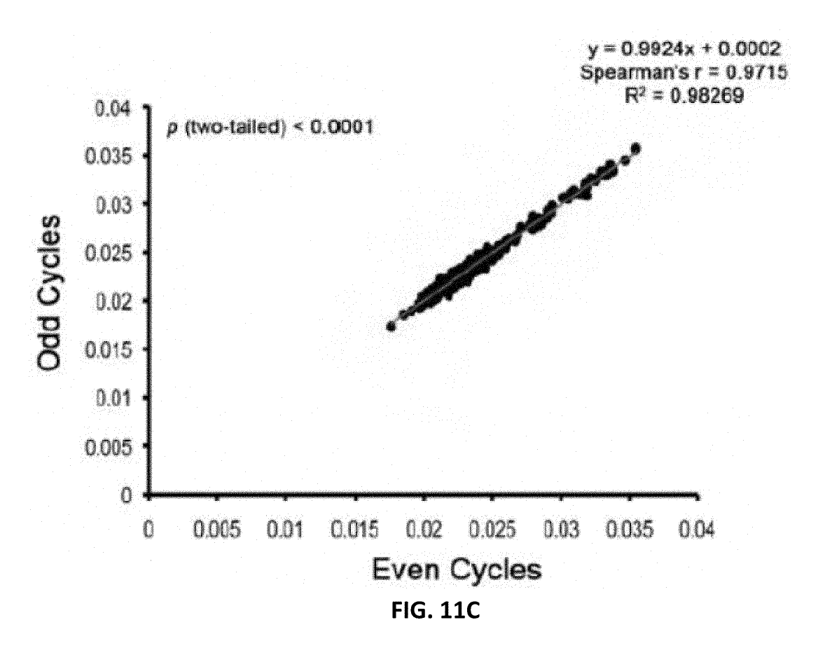

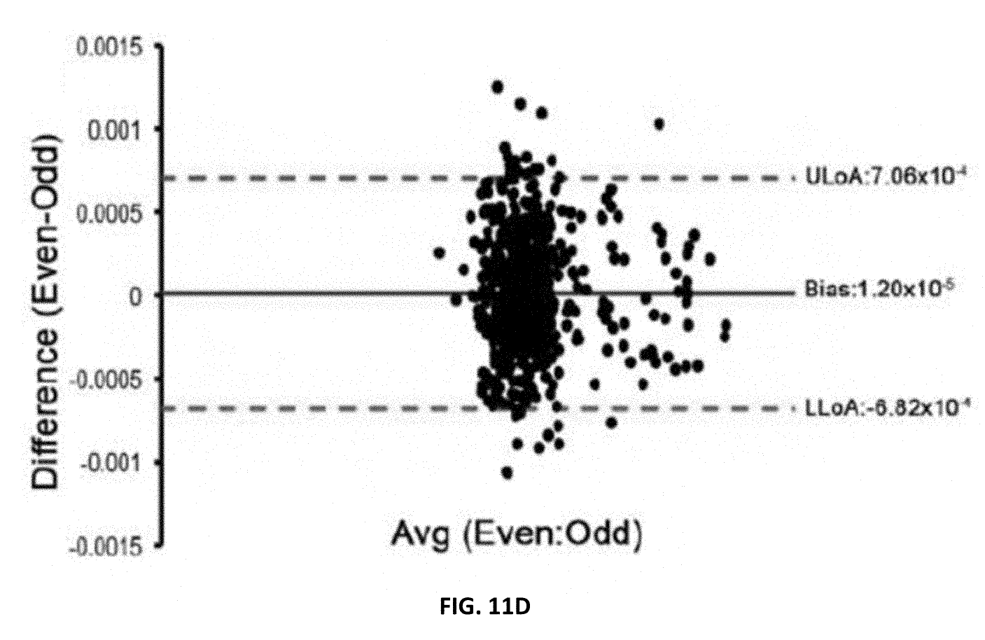

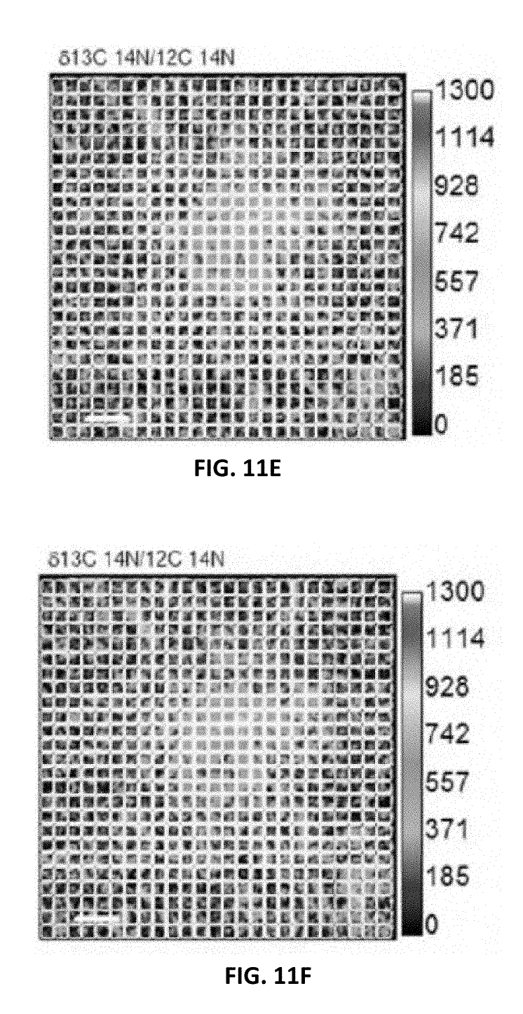

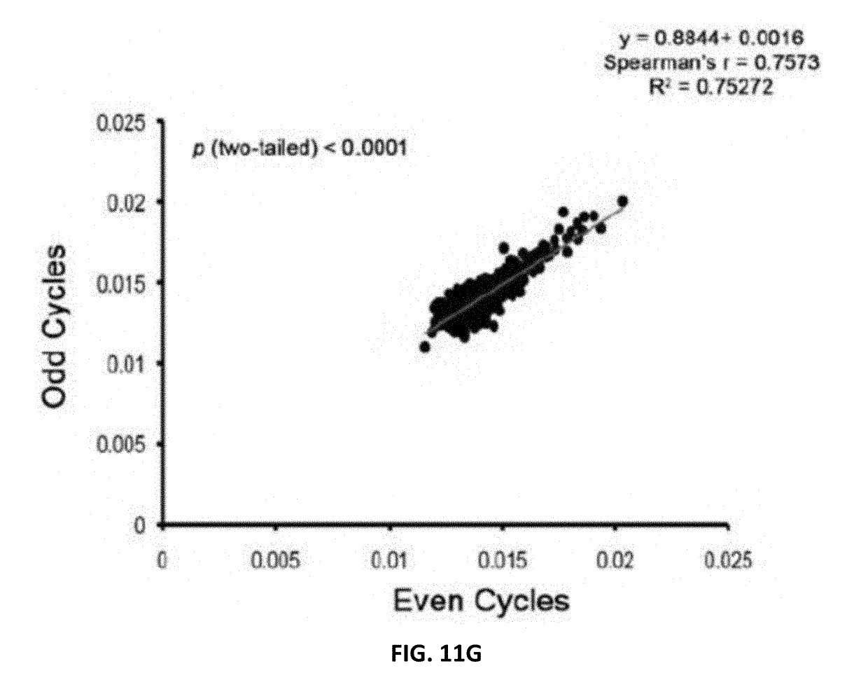

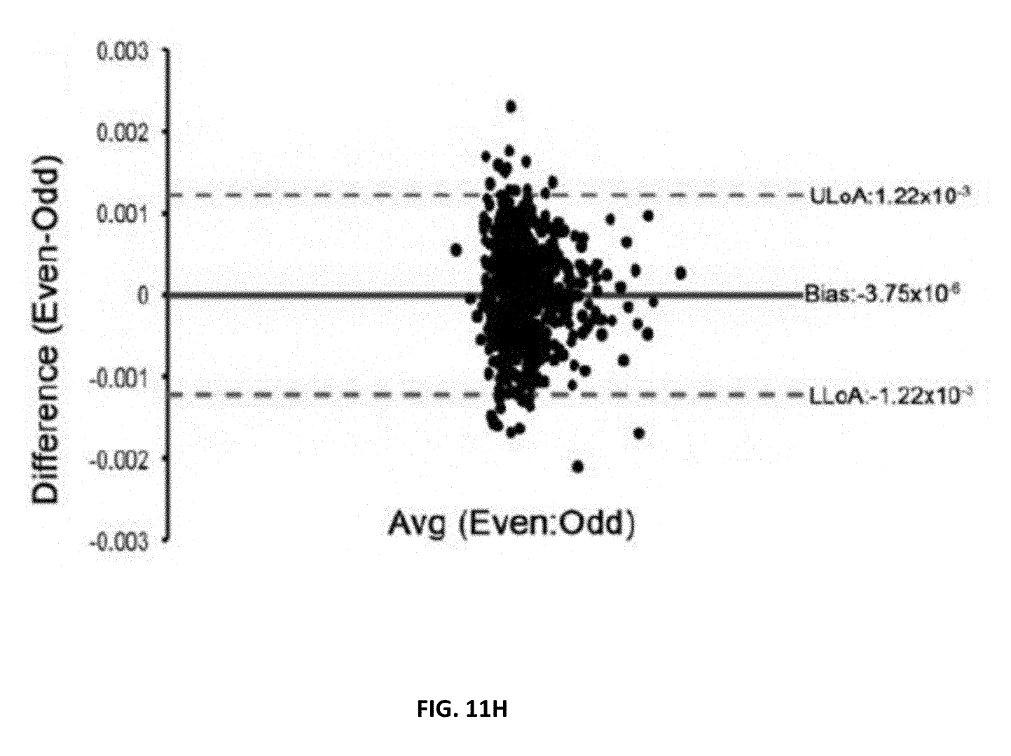

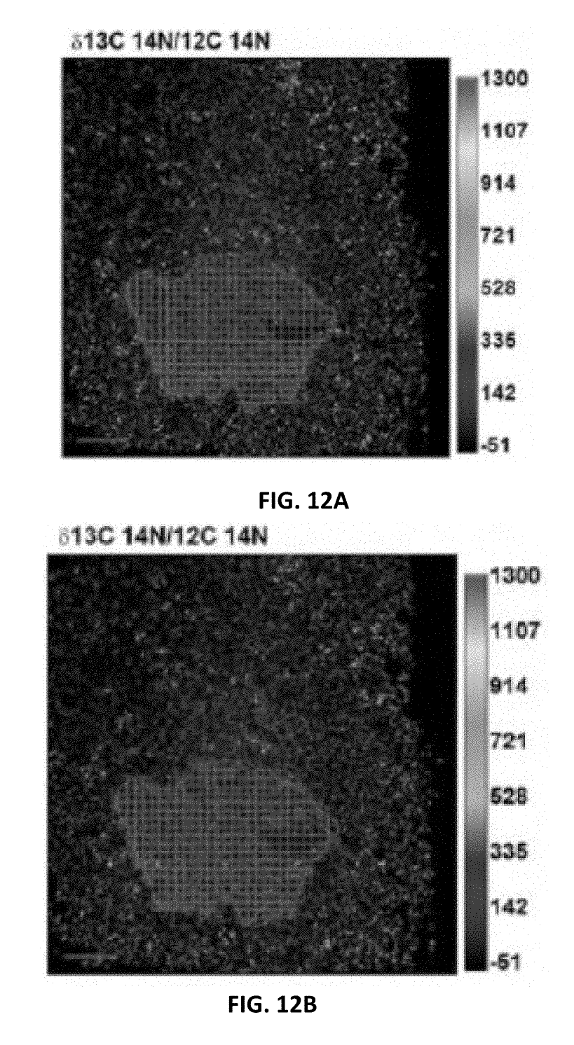

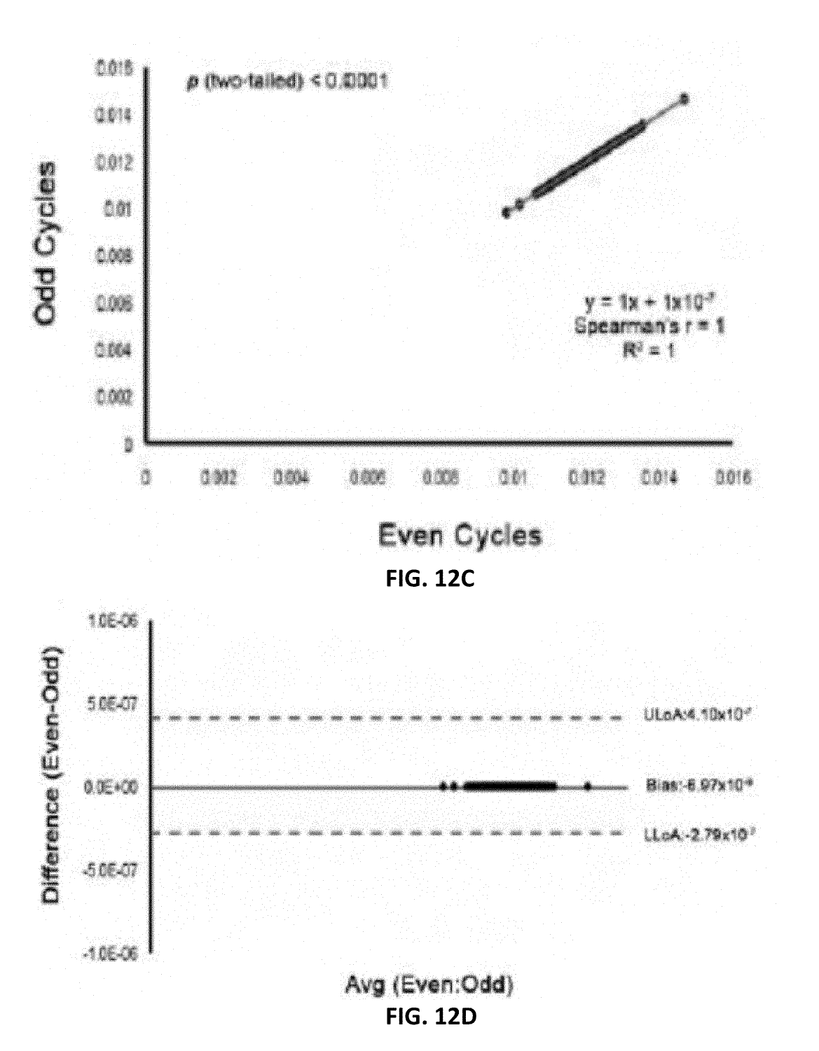

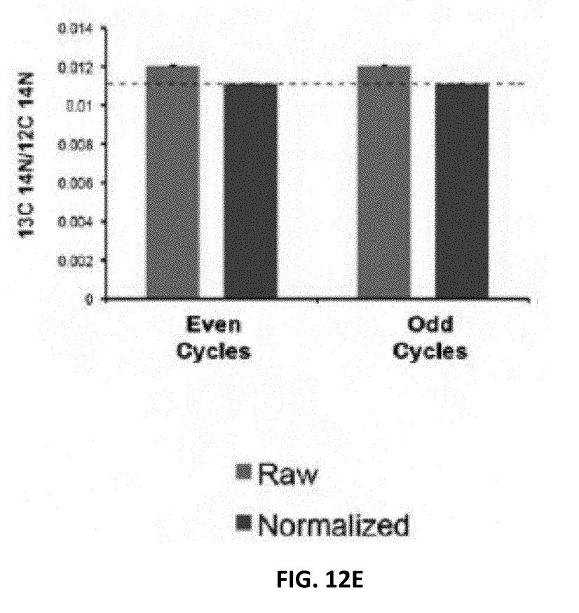

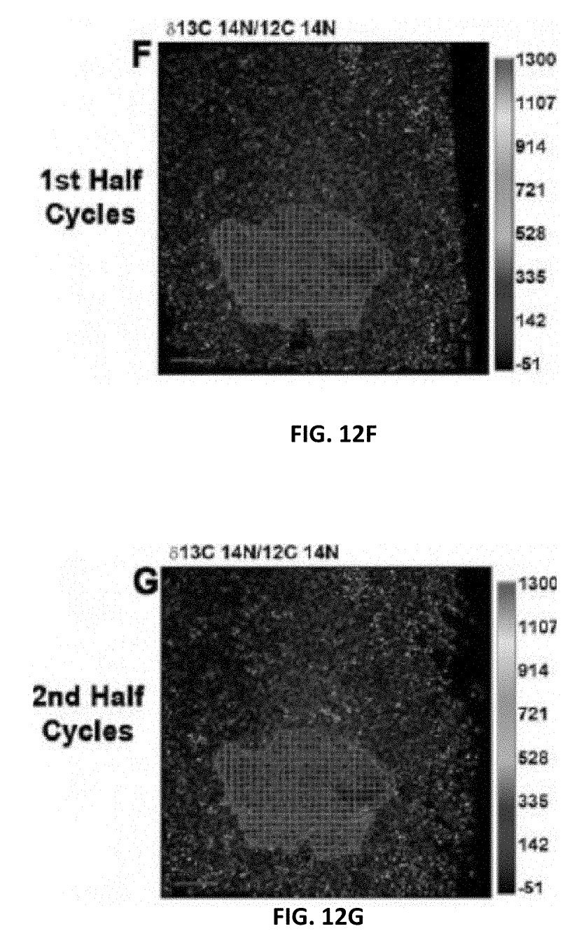

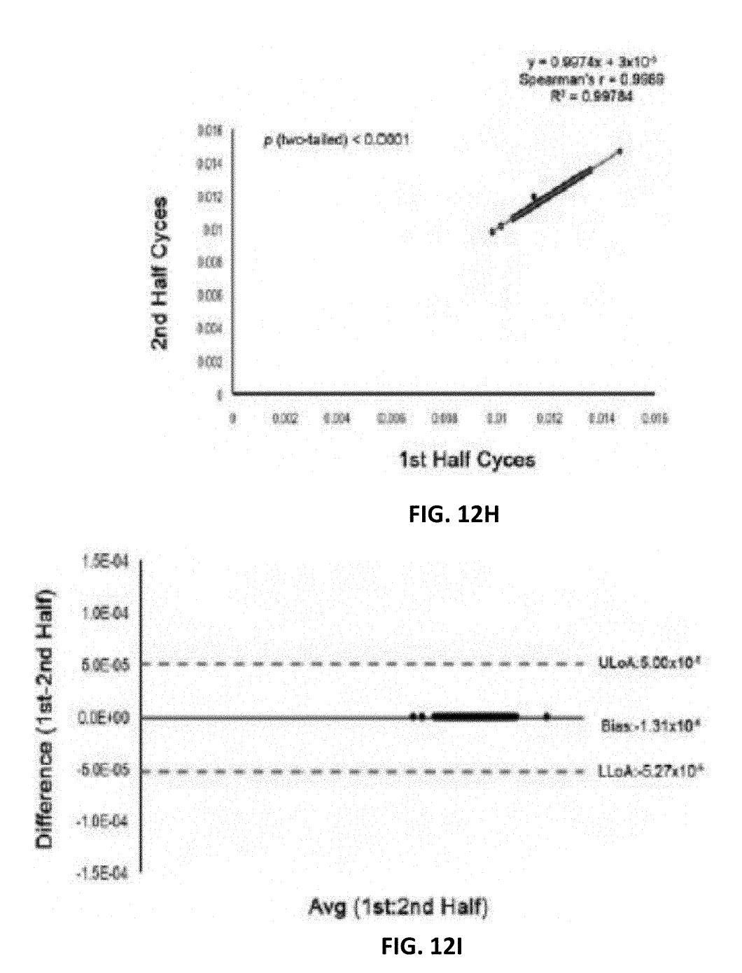

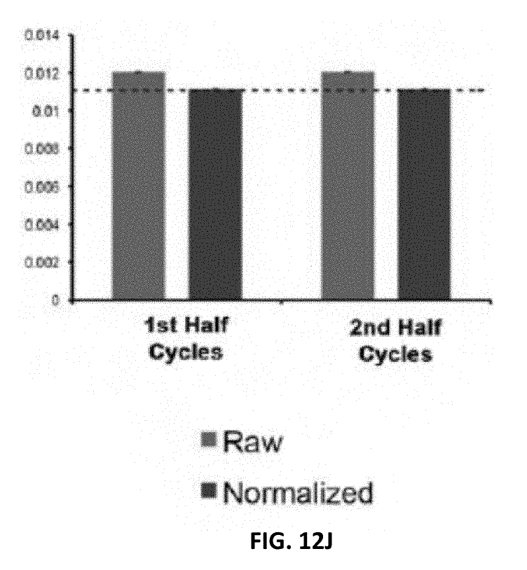

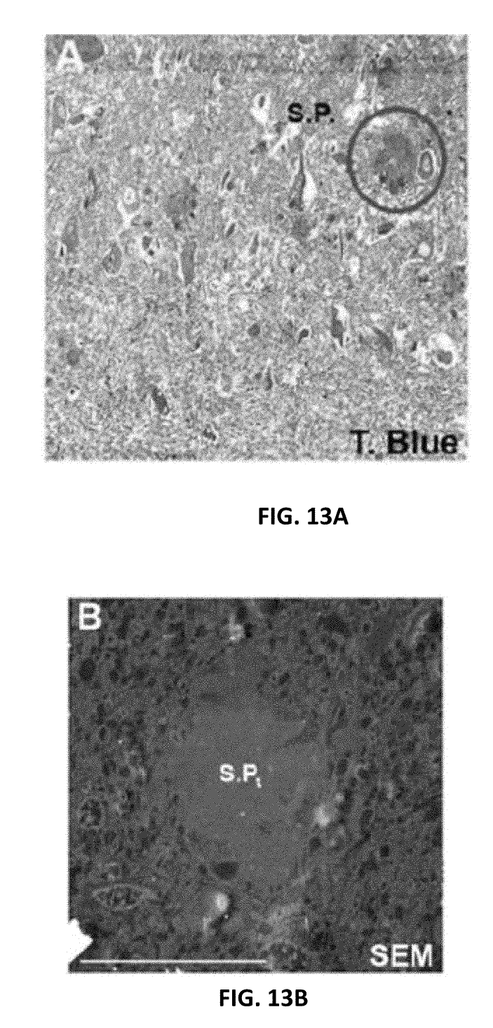

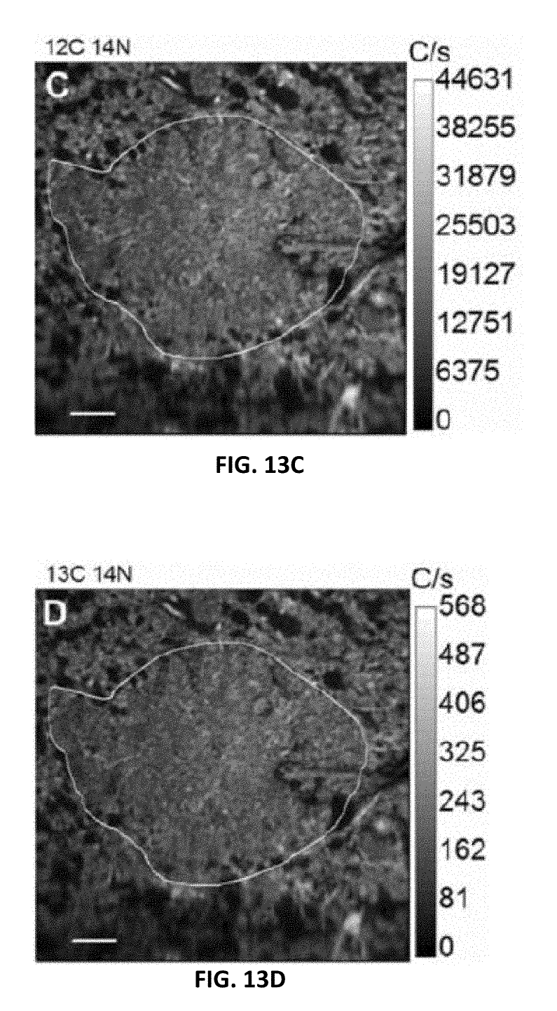

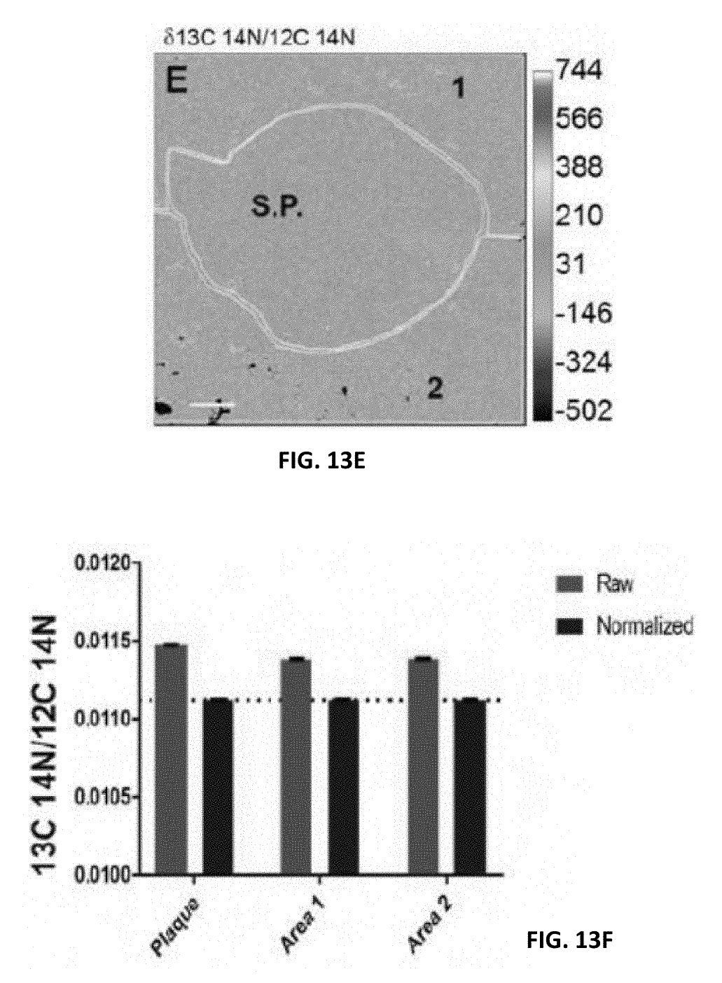

In order to study the variability in SILK-SIMS imaging, test-retest analyses were conducted on the mouse and human images. However, because this technique is destructive, the ROIs chosen for imaging will not remain the same over the course of two separate, individual measurements. Therefore, the isotope variability within the cycles of individual images was measured as data acquisition can take between 1-18 hrs depending on the level of isotopic enrichment and precision needed. There was no significant difference in the test re-test reliability ¹³C¹⁴N⁻/¹²C¹⁴N⁻ ratios of individual features in APP/PS1 mouse brain (FIG. 10). The test-retest-reliability coefficient of the pixel-to-pixel correlation between image cycles over time showed high reliability (neuron, 1.1 hr acquisition, Spearman's r=0.9715, p<0.0001; plaque, 0.91 hr acquisition, Spearman's r=0.7573, p<0.0001). Bland-Altman²² test-retest-reliability also demonstrated a high level of agreement with the isotope images on a per pixel basis over time, as nearly all fell within a 95% confidence interval (FIG. 11; TABLES 2-5). However, to obtain the level of precision and accuracy seen in the standard curve (FIG. 1) and animal labeling (FIG. 2, FIG. 7, FIG. 9, FIG. 11) paradigms, particularly when low enrichment is expected (e.g., human samples), overnight measurements are required. In such cases the need to ascertain the reliability of test results over time—test-retest reliability—is paramount. The pixel-to-pixel test re-test reliability comparing even and odd cycles as well as the first and last 20 cycles (˜6 hrs each) of the 40 cycle image of an unlabeled human Aβ plaque feature (FIGS. 12A-12C, and FIGS. 12F-12H) showed very high coefficients of stability (even vs odd, Spearman's r=1, p<0.0001; 1^{st} half vs 2^{nd} half, Spearman's r=0.9989, p<0.0001). Likewise, all pixel-to-pixel comparisons fell within a 95% confidence interval (FIG. 13D, FIG. 13I; TABLE 2-5) and no significant difference in the ¹³C¹⁴N⁻/¹²C¹⁴N⁻ ratios was present (FIG. 12E, FIG. 12J) demonstrating our results are consistent over time. We focused on the plaque feature instead of the whole image in order to avoid the astigmatism apparent at the edges of larger (80 μm) images such as this one.

TABLE 2
Bland-Altman plot statistics for SILK-SIMS imaging of neuron

| | | | | T value | | | |
|---|---|---|---|---|---|---|---|
| | | | | for 624 | | | |
| | | Standard | | degrees | | | |
| | | Error | Standard | of | Confidence | Confidence intervals | |
| Parameter | Unit | Formula | error (se) | freedom | (se * t) | from | to |
| Number | 625 | | | | | | |
| (n) | | | | | | | |
| Degrees | 624 | \sqrt{s2/n} | 1.41548E−05 | 1.96 | 2.77434E−05 | −1.57849E−05 | 3.97018E−05 |
| of | | | | | | | |
| freedom | | | | | | | |
| (n − 1) | | | | | | | |
| Difference | 1.19584E−05 | | | | | | |
| mean | | | | | | | |
| ( [symbol] ) | | | | | | | |
| Standard | 0.00035387 | | | | | | |
| deviation | | | | | | | |
| (s) | | | | | | | |
| [symbol] − 1.96 s | −0.000681626 | \sqrt{3s2/n} | 2.45168E−05 | 1.96 | 4.80529E−05 | −0.000729679 | −0.000633573 |
| [symbol] + 1.96 s | 0.000705543 | \sqrt{3s2/n} | 2.45168E−05 | 1.96 | 4.80529E−05 | 0.00065749 | 0.007533596 |

TABLE 3
Bland-Altman plot statistics for SILK-SIMS imaging of plaque

| | | | | T value | | | |
|---|---|---|---|---|---|---|---|
| | | | | for 624 | | | |
| | | Standard | | degrees | | | |
| | | Error | Standard | of | Confidence | Confidence intervals | |
| Parameter | Unit | Formula | error (se) | freedom | (se * t) | from | to |
| Number | 625 | | | | | | |
| (n) | | | | | | | |
| Degrees | 624 | \sqrt{s2/n} | 2.49069E−05 | 1.96 | 4.88174E−05 | −5.25646E−05 | 4.50702E−05 |
| of | | | | | | | |
| freedom | | | | | | | |
| (n − 1) | | | | | | | |
| Difference | −3.74719E−06 | | | | | | |
| mean | | | | | | | |
| ( [symbol] ) | | | | | | | |
| Standard | 0.000622671 | | | | | | |
| deviation | | | | | | | |
| (s) | | | | | | | |
| [symbol] − 1.96 s | −0.001224183 | \sqrt{3s2/n} | 4.31399E−05 | 1.96 | 8.45543E−05 | −0.001308738 | −0.001139629 |
| [symbol] + 1.96 s | 0.001216689 | \sqrt{3s2/n} | 4.31399E−05 | 1.96 | 8.45543E−05 | 0.001132135 | 0.001301243 |

TABLE 4
Bland-Altman plot statistics for SILK-SIMS imaging of unlabeled
human AD plaque (Even vs Odd)

| | | | | T value | | | |
|---|---|---|---|---|---|---|---|
| | | | | for 624 | | | |
| | | Standard | | degrees | | | |
| | | Error | Standard | of | Confidence | Confidence intervals | |
| Parameter | Unit | Formula | error (se) | freedom | (se * t) | from | to |
| Number | 393 | | | | | | |
| (n) | | | | | | | |
| Degrees | 392 | \sqrt{s2/n} | 6.97455E−09 | 1.968 | 1.37259E−08 | −2.07005E−08 | 6.75137E−09 |
| of | | | | | | | |
| freedom | | | | | | | |
| (n − 1) | | | | | | | |
| Difference | −6.97455E−09 | | | | | | |
| mean | | | | | | | |
| ( [symbol] ) | | | | | | | |
| Standard | 1.38265E−07 | | | | | | |
| deviation | | | | | | | |
| (s) | | | | | | | |
| [symbol] − 1.96 s | −2.7908E−07 | \sqrt{3s2/n} | 1.20803E−08 | 1.968 | 2.3774E−08 | −3.02854E−07 | −2.55306E−07 |
| [symbol] + 1.96 s | 4.10371E−07 | \sqrt{3s2/n} | 1.20803E−08 | 1.968 | 2.3774E−08 | 3.86597E−07 | 4.34145E−07 |

TABLE 5
Bland-Altman plot statistics for SILK-SIMS imaging of unlabeled
human AD plaque (First vs Second Half)

| | | | | T value | | | |
|---|---|---|---|---|---|---|---|
| | | | | for 624 | | | |
| | | Standard | | degrees | | | |
| | | Error | Standard | of | Confidence | Confidence intervals | |
| Parameter | Unit | Formula | error (se) | freedom | (se * t) | from | to |
| Number | 393 | | | | | | |
| (n) | | | | | | | |
| Degrees | 393 | \sqrt{s2/n} | 1.31631E−06 | 1.96 | 2.59051E−06 | −3.90179E−06 | 1.27923E−06 |
| of | | | | | | | |
| freedom | | | | | | | |
| (n − 1) | | | | | | | |
| Difference | −1.31128E−06 | | | | | | |
| mean | | | | | | | |
| ( [symbol] ) | | | | | | | |
| Standard | 2.60949E−06 | | | | | | |
| deviation | | | | | | | |
| (s) | | | | | | | |
| [symbol] − 1.96 s | −5.26661E−05 | \sqrt{3s2/n} | 2.27992E−06 | 1.96 | 4.48689E−06 | −5.7153E−05 | −4.81792E−05 |
| [symbol] + 1.96 s | 5.00435E−05 | \sqrt{3s2/n} | 2.27992E−06 | 1.96 | 4.48689E−06 | 4.55566E−05 | 5.45304E−05 |

Aβ plaques from human AD brain were examined (TABLE 6).

TABLE 6
Patient demographics for SILK-SIMS imaging of Aβ plaques.

| | Amyloid | | | | | Δ between |
|---|---|---|---|---|---|---|
| | Status | | | | | labeling and |
| Participant | PET-PiB | CDR^{(a)} | AD Dementia | MMSE^{(a)} | Gender | DOE (Days) |
| 2 | pos | 1 | Mild | 22 | M | 1150 and 8^{(b)} |
| 5 | pos | 1 | Mild | 11 | M | 1648 |
| A40 | N/A | N/A | N/A | N/A | M | N/A^{(c)} |

CDR, Clinical Dementia rating;
MMSE Mini Mental State Exam;
DOE Date of Expiration
^{(a)}CDR and MMSE as most recent
^{(b)}This participant was in two SILK studies and thus has two time lapses
^{(c)}This participant was not part of the SILK studies

It is currently believed that the low contrast observed in ¹²C images of eponembedded samples indicates that sputtering rates are evenly distributed across the field analyzed and that implanted Cs⁺ concentrations are approximately equivalent throughout the sample from Pt2 who was labeled for 9 hrs with ¹³C₆-leucine in a previously completed in a SILK study, but passed away 8 days later for unrelated reasons.

¹³C₆ labeling was shown to be intercalated and in the periphery of a plaque subsection. This demonstrates for the first time, ¹³C₆-leucine labeling in vivo in human AD plaque. In summary, we have established proof-of-principle for using this method to quantify turnover of human AD plaques.

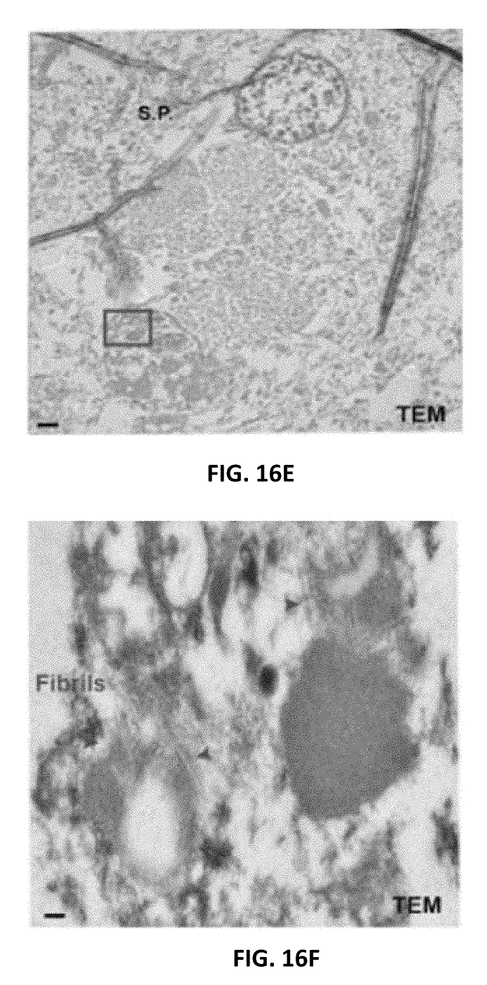

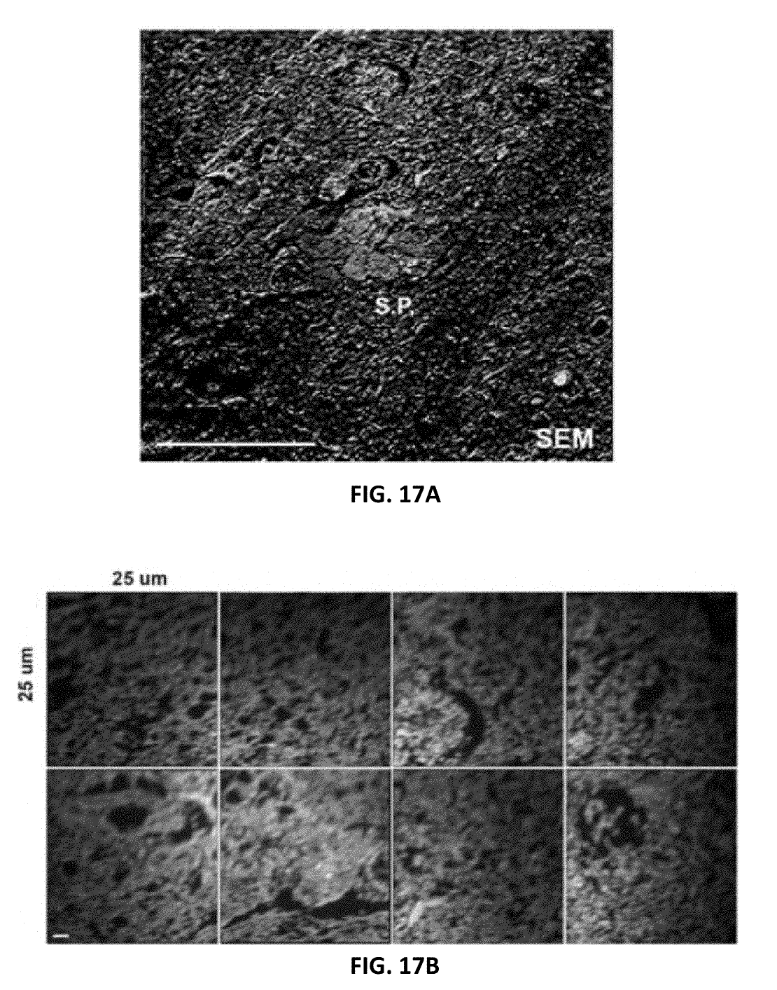

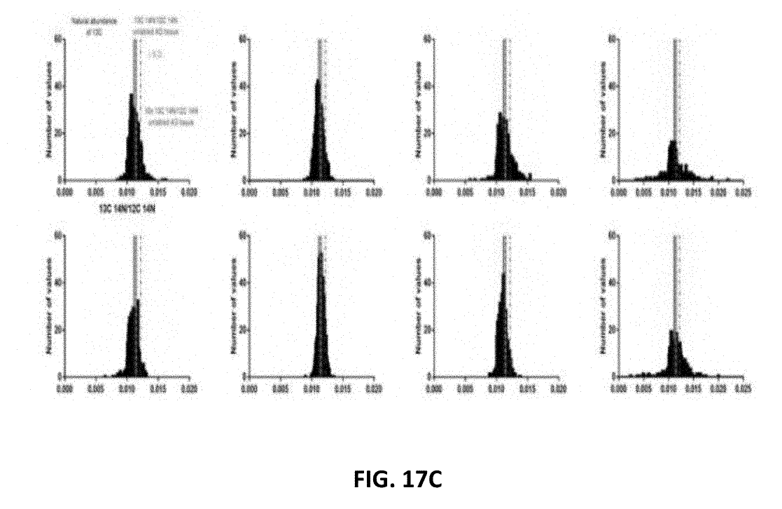

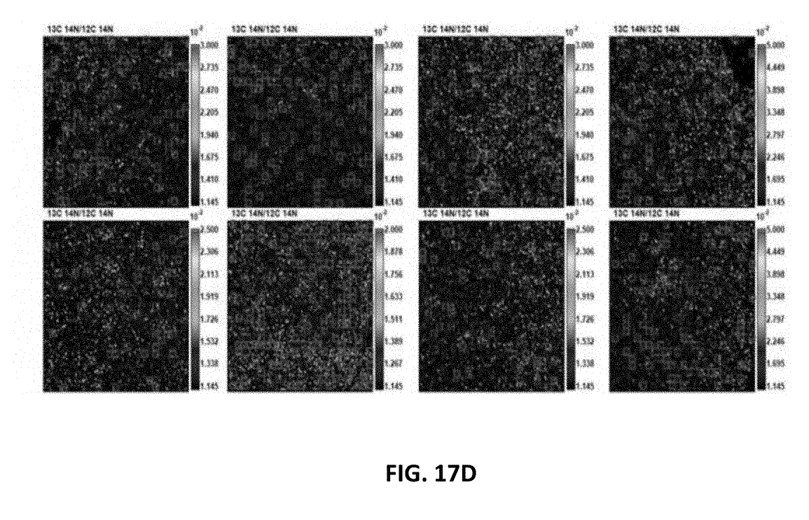

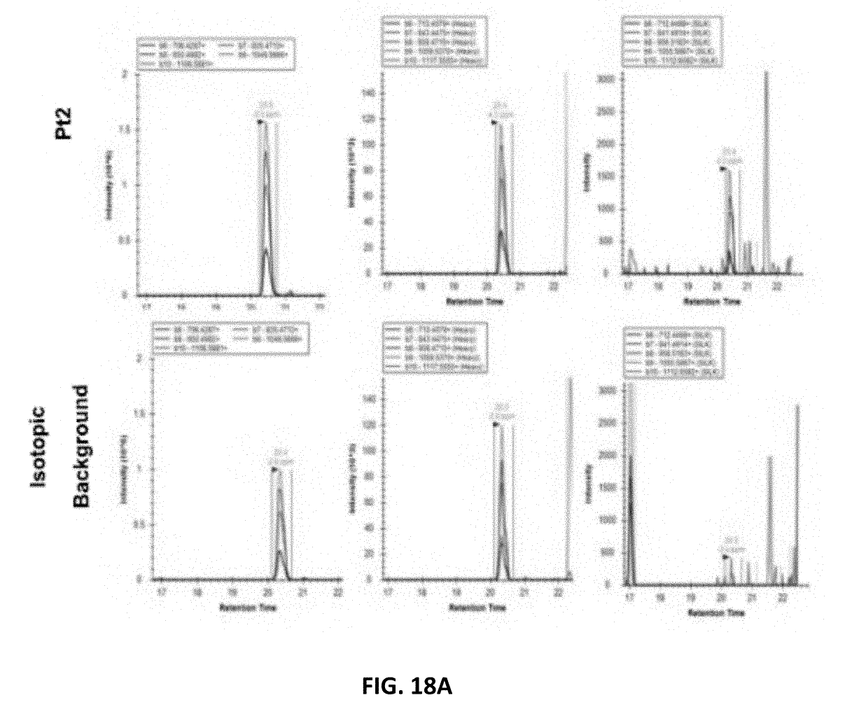

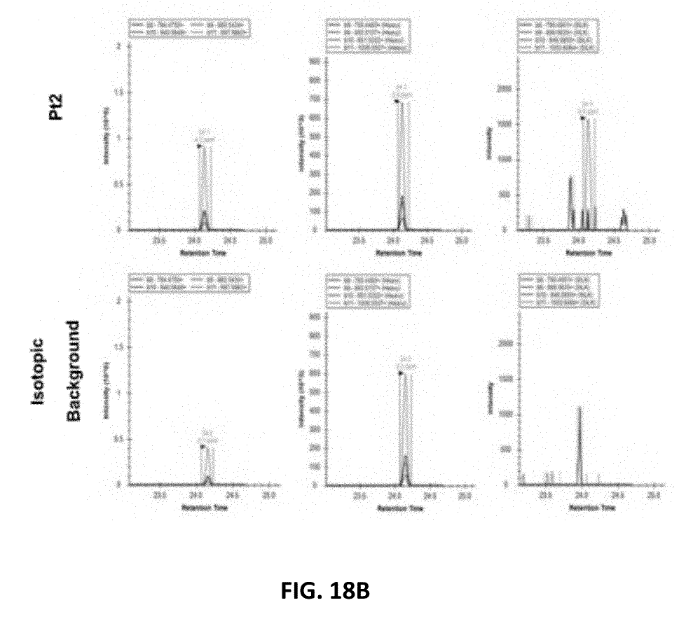

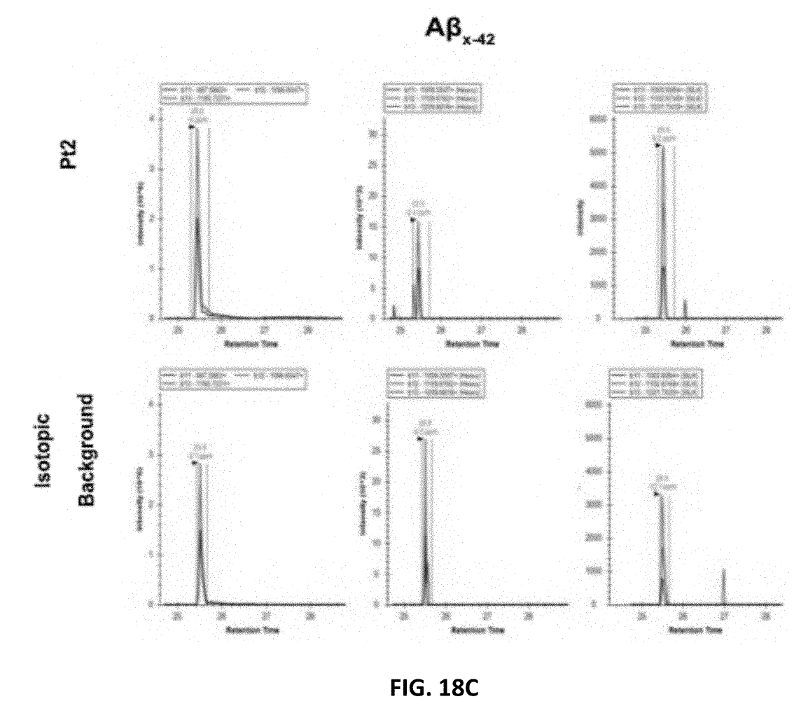

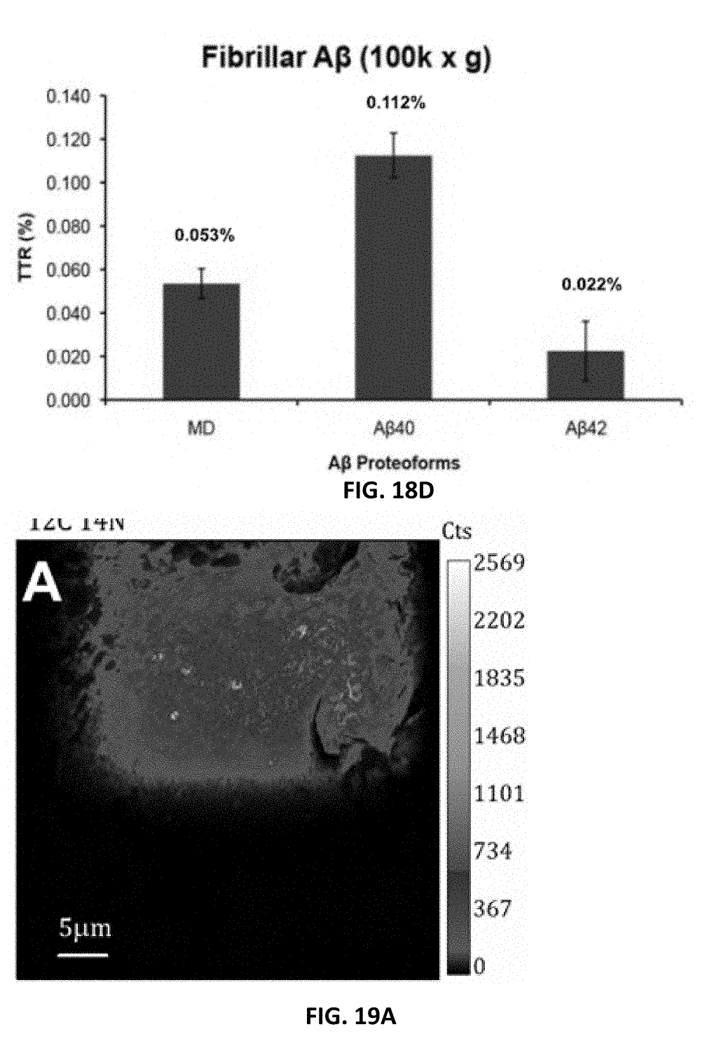

Lastly, we used bottom-up proteomics and targeted nLC-MS/MS to identify Aβx-38, 40, and 42 proteoforms^{23,24} and the Aβ mid-domain from the insoluble high molecular weight Aβ aggregates²⁴ to provide orthogonal validation of the presence of ¹³C₆-leucine labeled peptides within Pt2 brain. We were unable detect ¹³C₆-leucine labeled Aβx-38, but Aβx-40, 42, and mid-domain peptides were enriched at 0.112%, 0.022%, and 0.053% above isotopic background, respectively (FIG. 17). These results validate the ¹³C¹⁴N⁻/¹²C¹⁴N⁻ enrichment identified as discrete punta throughout the plaques in human AD brain and provides molecular identity to some of the ¹³C enrichment seen in Pt2.

In summary, plaques are not as static as previously thought. There still appears to be active accumulation of protein into the plaque periphery. These studies have implications for PET-PiB and imaging and tracking of AD generally.

Abbreviations

C⁻: Carbon ion

CN⁻: Cyanide ion

Cts/s: counts/second

HMW: High Molecular weight

Hrs: Hours

IP: Immunoprecipitation

MTBSTFA: N-Methyl-N-tert-butyldimethylsilyltrifluoroacetamide

ms: Millisecond

nm: Nanometer

NanoSIMS: Nanoscale secondary ion mass spectrometry

Pt: Participant

px: Pixel(s)

ROI: Region of interest

s: Second

SILK: Stable isotopic labeling kinetics

TEABC: Triethylammonium bicarbonate

μm: Micron

### Example 1 References

1 Mawuenyega, K. G., Kasten, T., Sigurdson, W. & Bateman, R. J. Amyloid-beta isoform metabolism quantitation by stable isotope-labeled kinetics. Anal Biochem 440, 56-62, doi:10.1016/j.ab.2013.04.031 (2013).
2 Bateman, R. J. et al. Human amyloid-beta synthesis and clearance rates as measured in cerebrospinal fluid in vivo. Nat Med 12, 856-861, doi:10.1038/nm1438 (2006).
3 Mawuenyega, K. G. et al. Decreased clearance of CNS beta-amyloid in Alzheimer's disease. Science 330, 1774, doi:10.1126/science.1197623 (2010).
4 Patterson, B. W. et al. Age and amyloid effects on human central nervous system amyloid-beta kinetics. Ann Neurol, doi:10.1002/ana.24454 (2015).
Matveev, S. V. et al. A distinct subfraction of Aβ is responsible for the high-affinity Pittsburgh compound B-binding site in Alzheimer's disease brain. J Neurochem 131, 356-368, doi:10.1111/jnc.12815 (2014).
6 Hardy, J. A. & Higgins, G. A. Alzheimer's disease: the amyloid cascade hypothesis. Science 256, 184-185 (1992).
7 Mann, D. M., Marcyniuk, B., Yates, P. O., Neary, D. & Snowden, J. S. The progression of the pathological changes of Alzheimer's disease in frontal and temporal neocortex examined both at biopsy and at autopsy. Neuropathol Appl Neurobiol 14, 177-195 (1988).
8 Delaere, P., He, Y., Fayet, G., Duyckaerts, C. & Hauw, J. J. Beta A4 deposits are constant in the brain of the oldest old: an immunocytochemical study of 20 French centenarians. Neurobiol Aging 14, 191-194 (1993).
9 Bennett, D. A. et al. Pathological changes in frontal cortex from biopsy to autopsy in Alzheimer's disease. Neurobiol Aging 14, 589-596 (1993).
10 Giannakopoulos, P. et al. Tangle and neuron numbers, but not amyloid load, predict cognitive status in Alzheimer's disease. Neurology 60, 1495-1500 (2003).
11 Ingelsson, M. et al. Early Abeta accumulation and progressive synaptic loss, gliosis, and tangle formation in AD brain. Neurology 62, 925-931 (2004).
12 Katzman, R. et al. Clinical, pathological, and neurochemical changes in dementia: a subgroup with preserved mental status and numerous neocortical plaques. Ann Neurol 23, 138-144, doi:10.1002/ana.410230206 (1988).
13 Terry, R. D. et al. Physical basis of cognitive alterations in Alzheimer's disease: synapse loss is the major correlate of cognitive impairment. Ann Neurol 30, 572-580, doi:10.1002/ana.410300410 (1991).
14 Price, J. L. & Morris, J. C. Tangles and plaques in nondemented aging and “preclinical” Alzheimer's disease. Ann Neurol 45, 358-368 (1999).
Aizenstein, H. J. et al. Frequent amyloid deposition without significant cognitive impairment among the elderly. Arch Neurol 65, 1509-1517, doi:10.1001/archneur.65.11.1509 (2008).
16 Engler, H. et al. Two-year follow-up of amyloid deposition in patients with Alzheimer's disease. Brain 129, 2856-2866, doi:10.1093/brain/awl178 (2006).
17 Scheinin, N. M. et al. Follow-up of [11C]PIB uptake and brain volume in patients with Alzheimer disease and controls. Neurology 73, 1186-1192, doi:10.1212/WNL.0b013e3181 bacfl b (2009).
18 Villemagne, V. L. et al. Longitudinal assessment of Abeta and cognition in aging and Alzheimer disease. Ann Neurol 69, 181-192, doi:10.1002/ana.22248 (2011).
19 Kadir, A. et al. Dynamic changes in PET amyloid and FDG imaging at different stages of Alzheimer's disease. Neurobiol Aging 33, 198 el 91-114, doi:10.1016/j.neurobiolaging.2010.06.015 (2012).
20 Lechene, C. et al. High-resolution quantitative imaging of mammalian and bacterial cells using stable isotope mass spectrometry. J Biol 5, 20, doi:10.1186/jbiol42 (2006).
21 Alberts, B. et al. Molecular Biology of the Cell. 4rth edn, (Garland Science, 2002).
22 Giavarina, D. Understanding Bland Altman analysis. Biochem Med (Zagreb) 25, 141-151, doi:10.11613/BM.2015.015 (2015).
23 Smith, L. M., Kelleher, N. L. & Proteomics, C. f. T. D. Proteoform: a single term describing protein complexity. Nat Methods 10, 186-187, doi:10.1038/nmeth.2369 (2013).
24 Esparza, T. J. et al. Purification and Quantitative Characterization of Soluble HMW Amyloid-beta from Alzheimer's disease Brain Lysates. Scientific Reports, In PressScientific Reports 6, 38187, doi:10.1038/srep38187 (2016).

### Methods and Materials

Cell Culture.

A B-cell hybridoma line was grown for 5 days in leucine-free media that was supplemented with either ¹²C₆-leuince or ¹³C₆-leuince at 26 mg/L and mixed at the appropriate percentage of heavy isotope-containing media with 2% FBS. Cells were harvested and spun at 1,000 rpm for 5 min at room temperature (RT). Cell pellets resuspended in 4° C. Ringers wash solution for 5 minutes, spun, and then fixed with 4% paraformaldehyde in 100 mM NaCl, 30 mM HEPES, 2 mM CaCl₂, pH 7.2 (NaHCa) for 2 hrs. This was followed by three rinses of NaHCa at RT and the overnight incubation at 4° C. in NaHCa. Centrifugation was used throughout the following steps in order to re-concentrate the cells to a pellet. The following morning, pellets were placed into ddH₂O and then infiltrated with LR White Embedding Media (Catalog #14383, EMS, Hatfield, Pa.) using the manufacturer's published protocol with minor modification. Partial dehydration was accomplished by using 20%, EtOH 15 minutes, 40% EtOH 15 minutes, 50% EtOH 15 minutes, 70% EtOH 15 minutes, 85% EtOH 10 minutes, followed by 1 hour in a 2:1 LR White to 85% EtOH. Sections of LR White embedded samples were cut on a Leica UC7 Ultramicrotome, using a diamond knife. 200 nm and 400 nm sections were picked up with a perfect loop, placed on top of a polished silicon (Si) wafer (Catalog #534, University Wafer Inc., South Boston, Mass.) and let air dry on a 35° C. hot plate.

Animals.

Two male double transgenic mice expressing chimeric mouse/human amyloid precursor protein (Mo/HuAPP695swe) and a mutant human presenilin 1 (PS1-dE9) both directed to CNS neurons (stock 34832-JAX) were kindly provide by Dr. Timothy Miller. Animals were given leucine-free chow (Catalog #1831936, Test Diet, St. Louis, Mo.) with 5 mg/mL ¹²C₆-leucine added to 2% sucrose-containing drinking water to control leucine intake for a one week acclimation period. After the one-week acclimation period, animals were given 5 mg/mL ¹³C₆- or ¹²C₆-leucine orally via 2% sucrose drinking water, averaging 36 mL of H₂O/week (FIG. 2). Animals were 4 months old at the time of labeling and 6.5 months old at the end of labeling. Following the end of the labeling paradigm, animals were anesthetized with 65 mg/kg pentobarbital sodium and sacrificed by decapitation. Brains were removed and placed in 10% neutral-buffer formalin (Catalog #15740-01, EMS, Hatfield, Pa.). All animal procedures were conducted in accordance with the Washington University Animal Studies Committee, and are consistent with the National Institutes of Health (NIH) guidelines for the care and use of animals. Pieces of mouse brain were washed into NaHCa and incubated overnight at 4° C. The following morning, samples were stained with 1% osmium/NaHCa for 1 hour, washed 4 times over 1 hour and then en bloc stained with 1% uranyl acetate/H₂O for 1 hour in the dark. Samples were rinsed with 3 exchanges of water, 10 minutes each and then processed for LR White embedding as described above with the addition of being gold-coated once on the Si wafer. Serial adjacent sections were place on glass microscope slides for toluidine blue staining for light microscopy.

APP/PS1 Plasma Leucine.

Mouse whole blood was spun at 1,000×g for 10 min and the plasma (supernatant) removed. Plasma proteins were precipitated with ice-cold acetone followed by de-lipidation with hexane and the aqueous fraction was dried in vacuo¹. 1:1 MTBSTFA/acetonitrile was added and samples were incubated 70° C. for 30 min. Duplicate 1 μL injections were made into an Agilent 5973 MSD mass spectrometer using a 30 μm×0.25 mm DB-5MS column (Agilent Technologies). Electron impact ionization and selected ion monitoring were used to measure endogenous unlabeled leucine at m/z 200 (molecular ion minus C-1 as CO₂-tBDMS), and ¹³C₆-leucine (tracer) was measured at m/z 205 as an m+5 ion. The tracer to tracee ratio (TTR) is taken as the m+5/m+0 peak area ratio of the biological sample minus the m+5/m+0 ratio of a natural abundance leucine sample. The molar fraction of labeled leucine was calculated as: MFL=TTR/(1+TTR).

Human Tissue.

Human cortical tissue samples were obtained from the Charles F. and Joanne Knight Alzheimer's Disease Research Center (ADRC) at Washington University School of Medicine in Saint Louis, Mo. Cognitive status was determined with a validated retrospective postmortem interview with an informant to establish the Clinical Dementia Rating (CDR). We used frontal lobe tissue from a mildly demented Alzheimer's participant (CDR1, age=88 yrs). Brain from prospectively assessed individual was obtained at autopsy with a postmortem interval of 15 hours. At autopsy, the left hemisphere was fixed in 10% neutral buffered formalin and stored at room temperature until further preparation.

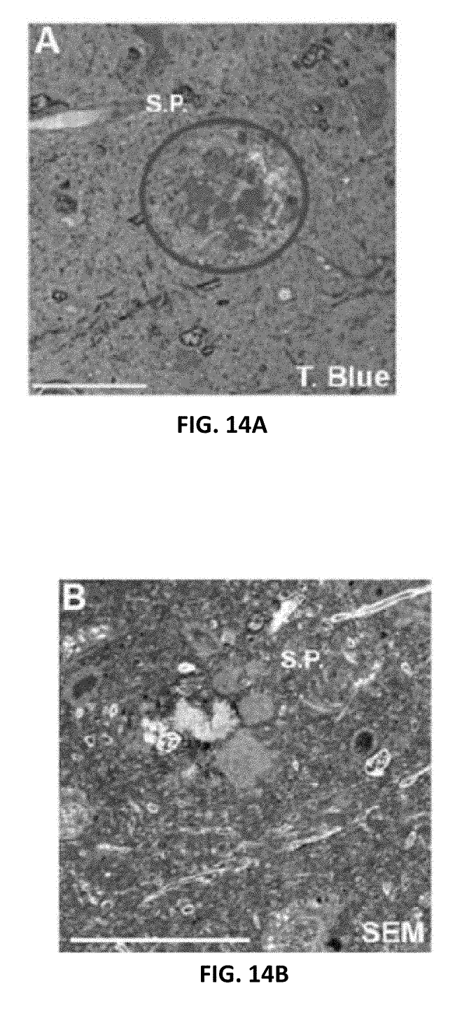

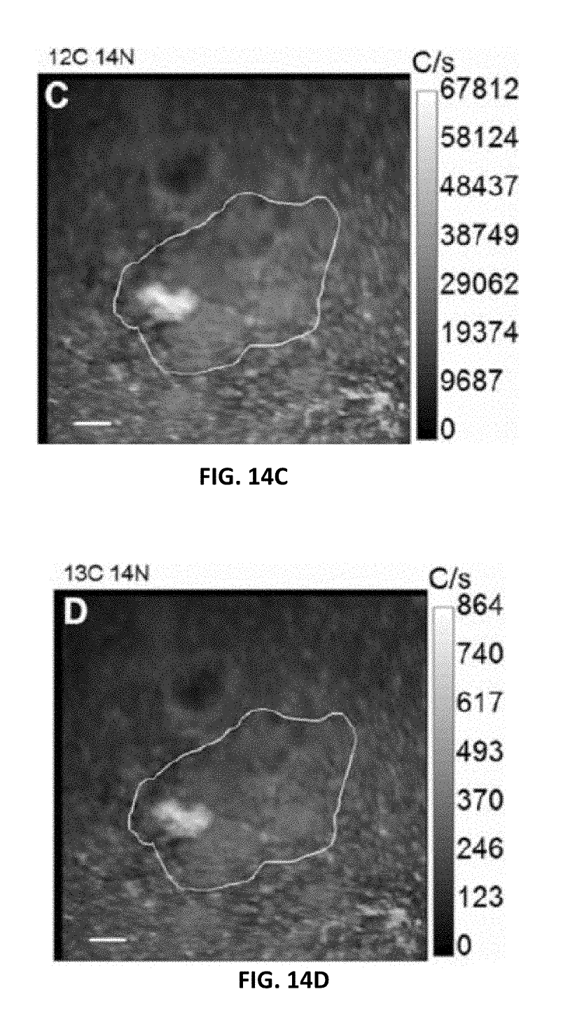

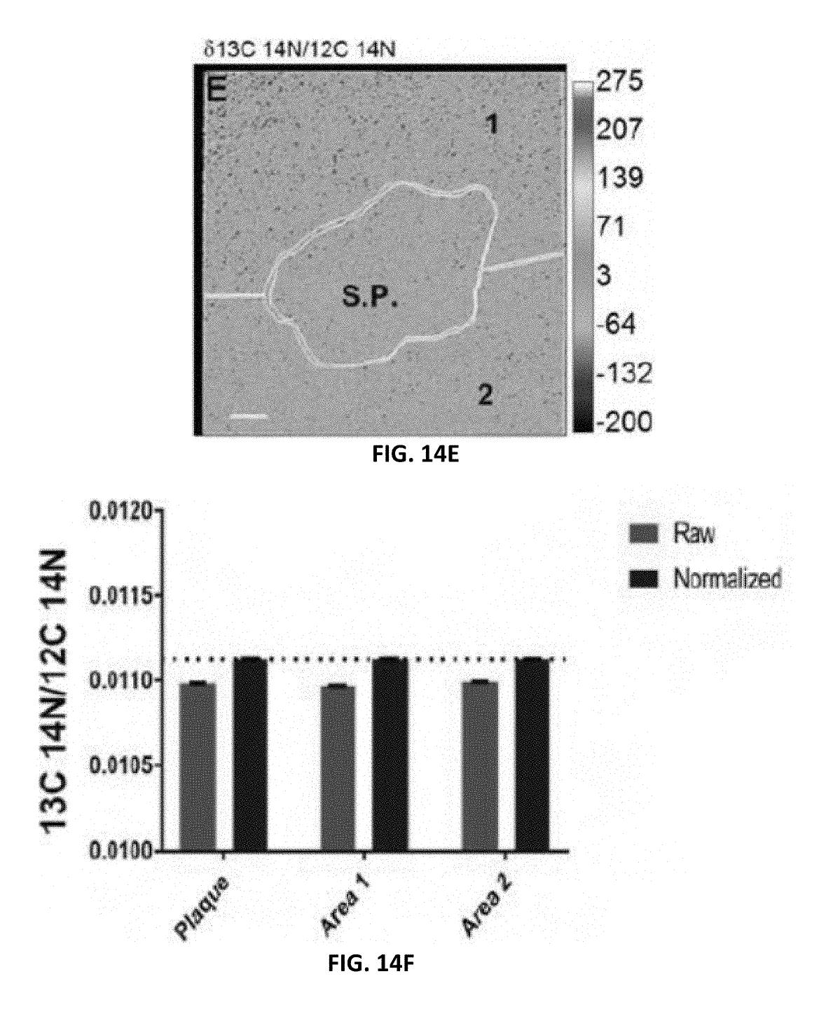

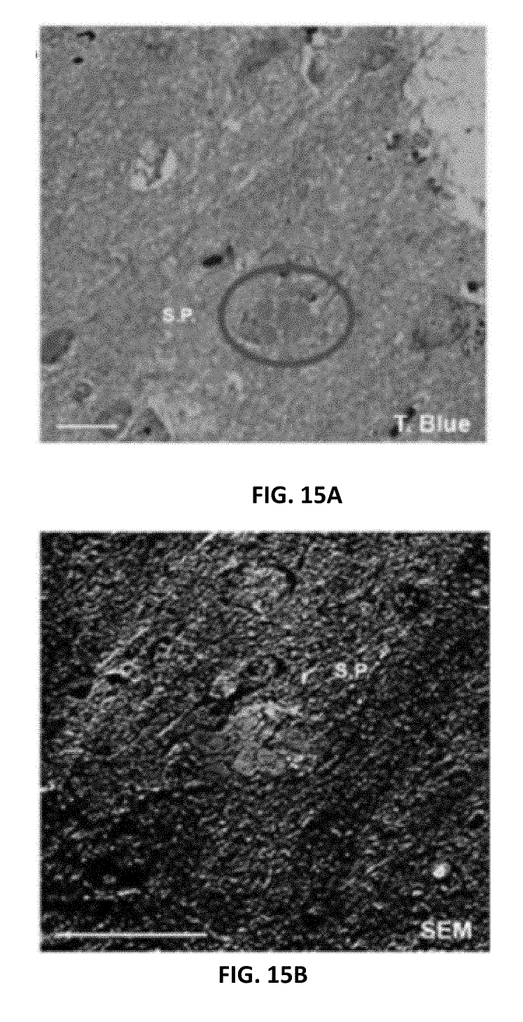

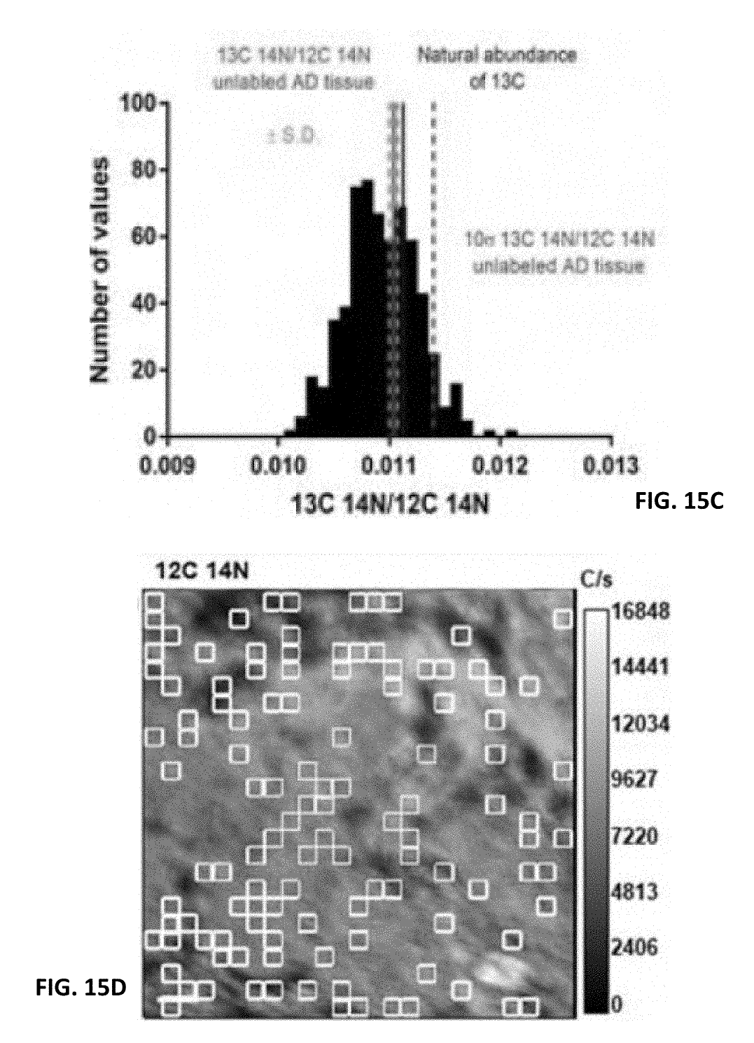

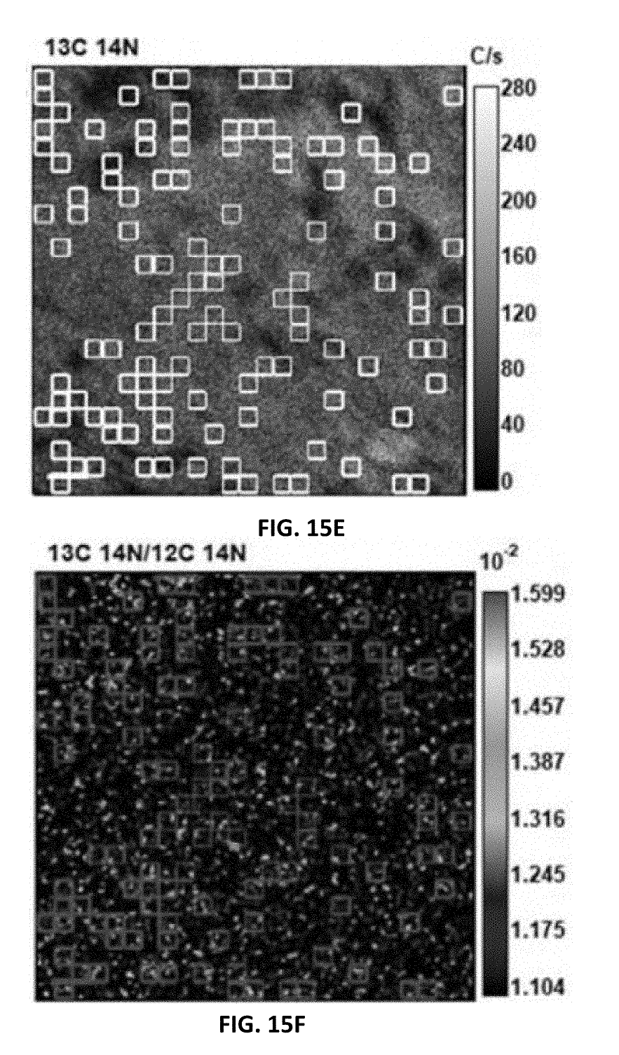

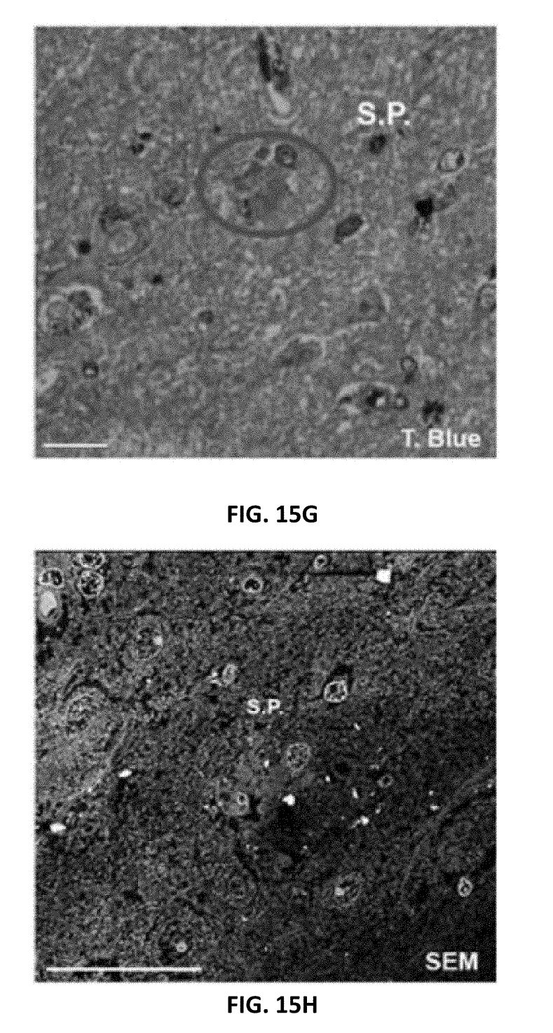

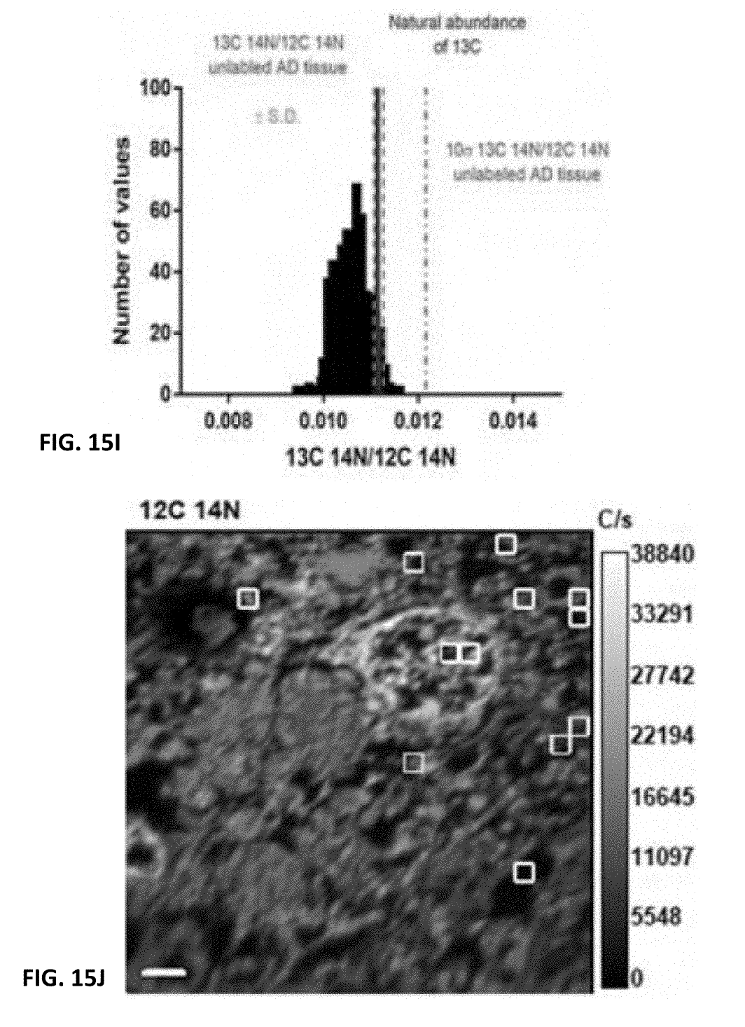

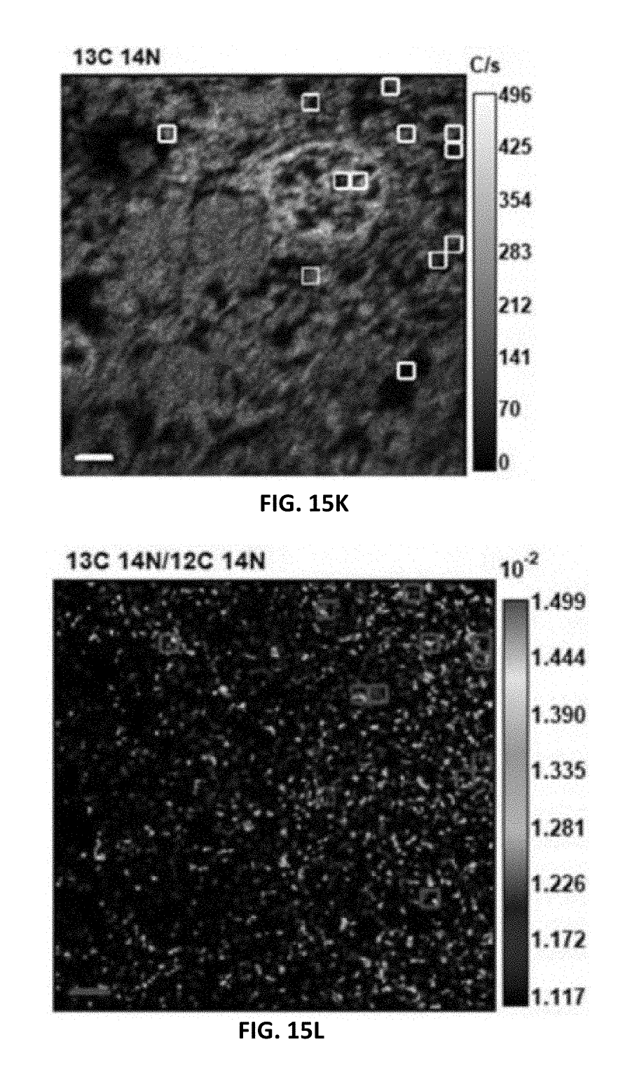

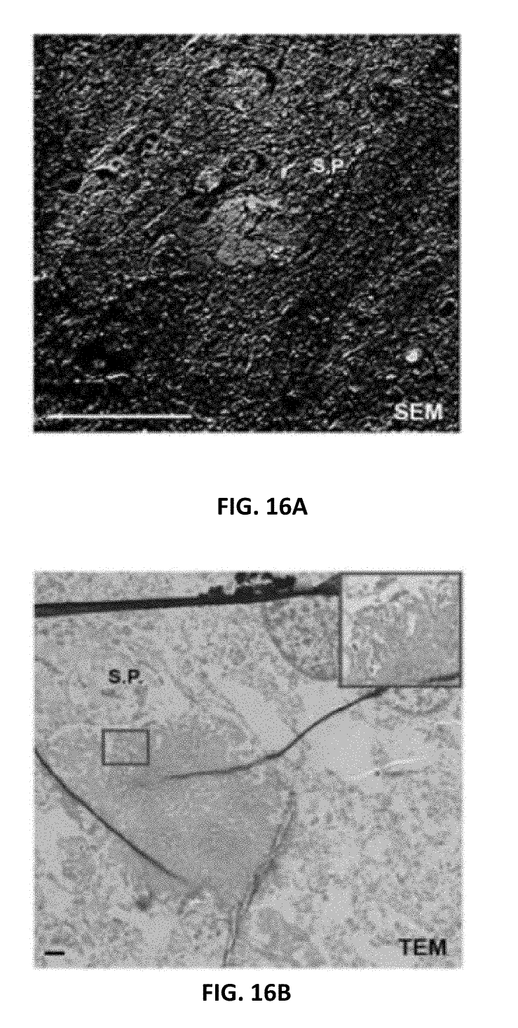

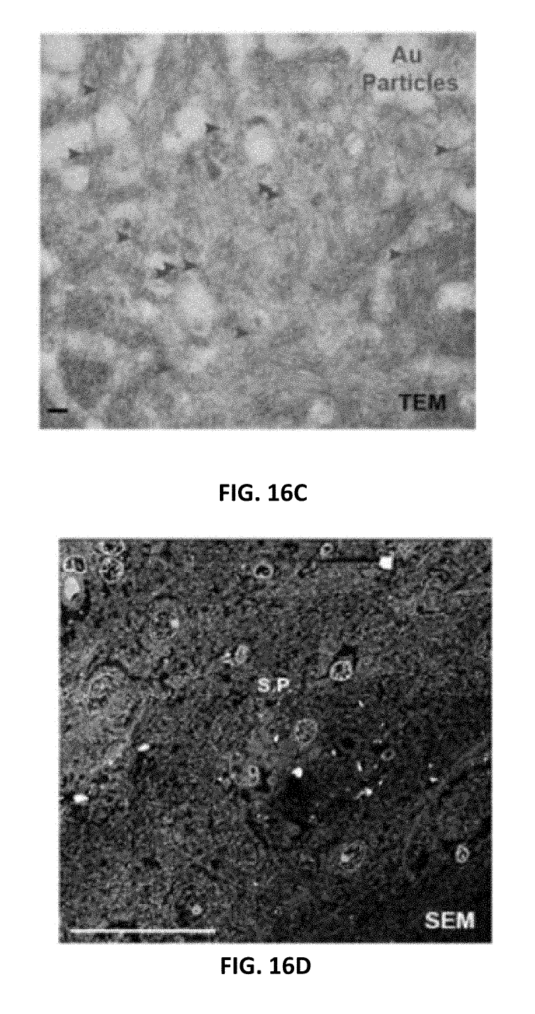

Frontal cortical tissue of PtA40 and Pt5 (FIG. 13-FIG. 14, respectively) were post-fixed in 2% osmium tetroxide in 0.1 M sodium cacodylate buffer for 1 hr., en bloc stained with 3% aqueous uranyl acetate for 1 hr., dehydrated in graded ethanols and embeded in PolyBed 812 catalog #08792-1 (Polysciences, Hatfield, Pa.). Blocks were polymerized at 80° C. for 72 hrs. Tissue blocks were sectioned using a diamond ultrathin section knife on a Reichert Ultra-Cut E ultramicrotome at 300-500 nm thick. Sections were transferred on a single polished Si wafer for SILK-SIMS analysis. Serial adjacent sections were place on glass microscope slides for toluidine blue staining for light microscopy. Tissue from frontal lobe of Pt2 was prepared and embedded in PolyBed 812 as described above for PtA40 and Pt5. The precuneus region of Pt2 was prepared and embedded in LR White as described above for animal tissue along with samples from the 10-week labeled APP/PS1 mouse (positive control) and PtA40 (negative control). Serial adjacent sections were place on glass microscope slides for toluidine blue staining for light microscopy.

Light Microscopy.

Toluidine blue stained sections were imaged with a Hamamatsu NanoZoomer 2.0-HT System at a maximum 40× objective. Imaging was done to guide feature identification and location for electron microscopy.

Electron Microscopy.

Images of the tissue and reference points were taken with a field emission scanning electron microscope (feSEM; Quanta™ 3D FEG, FEI, Hillsboro, Oreg.), in order to document plaque locations and provide an absolute coordinate system for the tissue. In-house coordinate transformation software was used to translate tissue ROIs and reference points found in the feSEM to the NanoSIMS instrument stage coordinate plane for relocation of the same ROIs. Additional sections were cut at 70-90 nm for transmission electron microscopy (JEOL JEM-1400Plus) to image selected plaques to define ultrastructure. Anti-Aβ antibody 83E1 (1:50) was used with goat anti-mouse secondary antibody conjugated to 10 nm gold particles (1:15).

MRI and PET-PiB Imaging.

Participants were labeled with the radiotracer N-methyl-²2-(4-methylaminophenyl)-6-hydroxybenzothiazole (PiB) for human brain PET imaging of amyloid deposition. PiB was prepared as previously described² and imaging performed on a Siemens 962 HR+ ECAT scanner as previously described³.

NanoSIMS.

Data was acquired on either a Cameca NanoSIMS 50 at WASHU (cells and human tissue) or NanoSIMS 50 L at Brigham and Women's Hospital (mouse tissue). Images of the B-cell hybridoma used to calculate that ¹³C₆-leucine standard curve were acquired at a 50 μm raster for 15 minutes at 1 ms/px and 65.5 s/plane (dwell time) for a total of 10 planes (i.e., cycles) per mass (256×256 px). APP/PS1 mouse brain tissue was acquired at a 17-60 μm raster for 11-87 minutes at 2 ms/px and 131 s/plane (dwell time) for a total of 5-40 cycles (i.e., planes) per mass (256×256 px).

Human.

Pt2 precuneus samples (embedded in LR White) were pre-sputtered at 30 μm raster at D1-1 aperture for 10 minutes followed by data acquisition at 25 μm raster at D1-2 aperture. Data acquisition was 2.5 hours at 5 ms/px and 327.68 s/plane (dwell time) for a total of 25 planes (i.e., cycles) per mass (256×256 px). PtA40, also embedded in LR White, was used as the negative control in this experimental set.

The Pt2 frontal lobe sample set with PtA40 (negative control) was embedded in PolyBed 812.

Unlabeled AD tissue (PtA40; FIG. 12-13) embedded in PolyBed812, was acquired at 45 μm raster for 11.6 hrs at 4 ms/px and 1048.576 s/plane (dwell time) for a total of 40 planes (i.e., cycles) per mass (512×512 px). Pt5 (FIG. 14) brain tissue was acquired at a 55 μm raster for 18 hrs at 5 ms/px and 1310 s/plane (dwell time) for a total of 40 planes (i.e., cycles) per mass (512×512 px).

NanoSIMS Data Analysis.

Each analysis was performed in 24-hour blocks with measurements on SiC standard to assess instrument stability followed by measurements on an un-labeled control prior to SILK-SIMS data acquisition. Raw image data was imported into custom, in-house particle definition software, L'Image, to produce quantitative mass images of heavy and light isotopes, and determine where any isotopic anomalies may be located. The fractional uncertainty, f, of the heavy/light isotope ratios in each region-of-interest (ROI) was calculated in Excel as the sum in quadrature of the standard deviation of the average ratios measured for non-labeled material, σ_{Std}, and the Poisson errors, σ_{ROI}, of the ROI itself, as given by the equation

$$f = {( \frac{{σ}_{Std}}{{R}_{Std}} )}^{2} + {( \frac{{σ}_{ROI}}{{R}_{ROI}} )}^{2} \qquad ( 1 )$$

where R̅_̅{̅S̅t̅d̅}̅ is the average ratio of repeated measurements on unlabeled tissue and R_{ROI} is the ratio calculated from summing the counts of every pixel contained within the individually defined ROI. This procedure represents the entire experimental precision and accuracy, including: counting statistics, matrix effects, systematic error, instrumental tuning, and differences between standards and samples. From this uncertainty, the amount, significance, and location of heavy isotopic labeling can be quantitatively determined. Using this analysis, SILK human tissue samples were thresholded and automatically parcellated into 10×10 pixel (976 nm²) ROIs. The heavy/light isotope ratios in each ROI with its standard deviation was calculated in Excel as the sum in quadrature of the standard deviation of the average ratios measured for non-labeled material as described above. The non-labeled material was PtA40, which was measured prior to the SILK-SIMS data acquisition. Those ROIs with The heavy/light isotope ratios≥ to the μ+2σ of the unlabeled sample were analyzed by one-way ANOVA followed by Dunnett's post hoc test.

Aβ Extraction.

1 g of frontal lobe tissue was homogenized in ice-cold 1×PBS with 0.05% CHAPS and centrifuged at 17,000×g for 30 min at 4° C. as previously described⁴. The supernatant was spun for 1 hr at 100,000×g at 4° C. and the resulting pellet solubilize in 5 M guanidine overnight at 4° C. with rotation. Next, samples were spun for 20 min at 17,000×g at 4° C. and the supernatant was diluted 1:10 in 1×PBS in BSA-block tubes as previously described⁴. Samples were immunoprecipitated (IP) with 50 uL of HJ5.1 anti-Aβ antibody (mid-domain epitope) coupled Dynabeads (Catalog #14311 D, Invitrogen, Carlsbad, Calif.) made following the manufacturer's instructions. After overnight incubation at 4° C. IPs were eluted in neat formic acid and dried in vacuo. Samples were resuspended in 50 mM TEABC (Catalog #17902, Sigma, St. Louis, Mo.), spiked with ¹⁵N-Aβ internal standard, and digested overnight with 0.25 ng/μL Lys-N(Catalog #100965-1, Seikagaku Biobusiness Corp., Tokyo, Japan) at 4° C.

Mass Spectrometry.

Samples were resuspended in 1% FA/10% ACN (v/v) with 20 nM BSA digest (Catalog #1863078, Pierce, Rockford, Ill.). Samples were analyzed in triplicate by nanoLC-MS/MS on an LTQ-Orbitrap Fusion (Thermo Fisher Scientific) in positive ion mode. Separations were performed using an online NanoAcquity UPLC (Waters, Milford, Mass.) using an ACQUITY UPLC HSS T3 (360 μm OD×75 μm ID) column packed with 10 cm C₁₈ (1.8 μm, 100 Å, Waters) at 300 nL/min and heated to 65° C. Mobile phases were 0.1% FA in water (A) and 0.1% FA in ACN (B). Samples were eluted from the column with the gradient starting at 12% B, which was ramped to 32% B over 10 min and further increased to 90% B over 5 min and held for 1 min, before re-equilibration to 12% B over 2 min. Total run time, including column equilibration, sample loading, and analysis was 30 min. The mass spectrometer was operated in targeted MS2 mode. MS2 spectra were acquired in the Orbitrap (30,000 at m/z 400) in centroid mode using XCalibur, version 4.0 (Thermo Fisher Scientific). Ion injection times for the targeted MS2 scans for labeled and unlabeled respectively (in ms) were: Aβ mid-domain (54, 1080), Aβ40 (54, 540), and Aβ42 (54, 1080). The Orbitrap automatic gain control targets were set 5×10⁵ for all proteoforms except Aβ42, which was set to 1×10⁶. The targeted precursor ions were sequentially isolated in the quadrupole and fragmented in the Orbitrap using HCD (isolation width 1.6 Da, normalized collision energy 25%, activation Q 0.250, and activation time 10 ms). The general mass spectrometric conditions were as follows: spray voltage 2.2 kV, 60% S-lens, and ion transfer tube temperature 275° C.

Mass Spectrometry Data Analysis.

Data (.raw files) were imported into a Skyline template containing the Lys-N C-terminal peptides of Aβ8, 40, 42 and the mid-domain. Retention time alignment was based on the 15N internal standard. The sum of all transitions (b ions monitored for each parent peptide) were summed for unlabeled and SILK Aβ peptides and exported from Skyline to Excel. The ratios of SILK/unlabeled of each replicate (triplicate injections) for Pt2 sample was taken followed by isotopic background subtraction of the mean ratio of an unlabeled participant to give the tracer-to-tracee ratio (TTR) minus background. Next, the background subtracted TTRs were used to calculate the mean and standard deviation (i.e., take the mean of the area ratios) of enrichment for each Aβ peptide.

### Methods and Materials References

1 Mittendorfer, B., Patterson, B. W. & Klein, S. Effect of weight loss on VLDL-triglyceride and apoB-100 kinetics in women with abdominal obesity. Am J Physiol Endocrinol Metab 284, E549-556, doi:10.1152/ajpendo.00379.2002 (2003).
2 Mathis, C. A. et al. Synthesis and evaluation of 11C-labeled 6-substituted 2-arylbenzothiazoles as amyloid imaging agents. J Med Chem 46, 2740-2754, doi:10.1021/jm030026b (2003).
3 Su, Y. et al. Quantitative analysis of PiB-PET with FreeSurfer ROIs. PLoS One 8, e73377, doi:10.1371/journal.pone.0073377 (2013).
4 Esparza, T. J. et al. Purification and Quantitative Characterization of Soluble HMW Amyloid-beta from Alzheimer's disease Brain Lysates. Scientific Reports, In Press (2016).

### Example 2: Silk-SIMS Imaging for Quantification of ¹³C₆-Leucine-Labeled Protein Deposition

This example shows in vivo stable isotope labeled kinetic images of Aβ plaques in AD brain utilizing SILK-SIMS imaging for quantification of ¹³C₆-leucine-labeled protein deposition.

This example utilizes in vivo incorporation of the stable isotope ¹³C₆-leucine into Aβ (and other proteins) in APP-PS1 mice by oral labeling and human AD patients via intravenous infusion. Following this, autopsied frontal cortex was prepared using established TEM protocols for 0.5 micron thick plastic-embedded sectioning and isotopically imaged by nanoscale secondary ion mass spectrometry (NanoSIMS) in a combined approach we term SILK-SIMS.

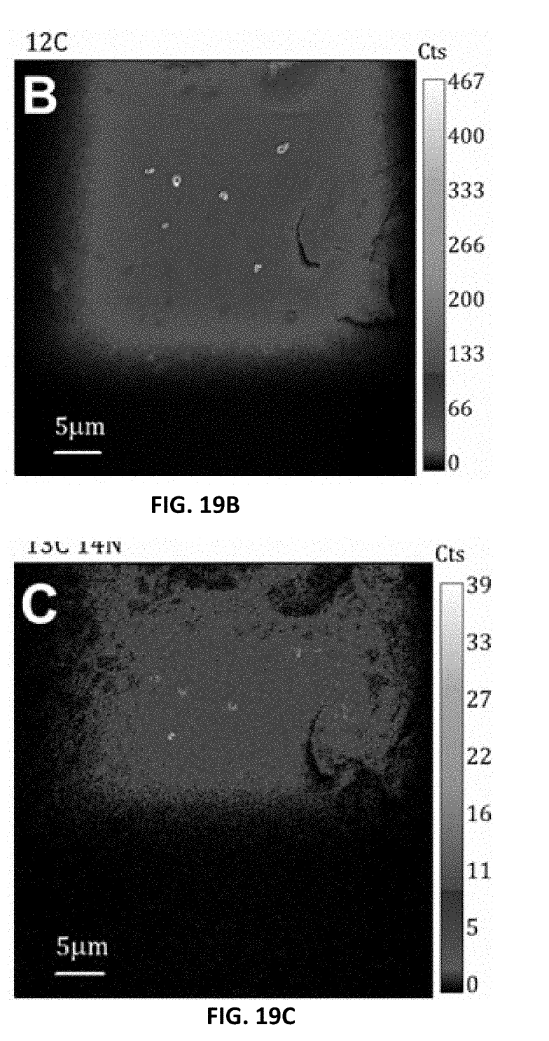

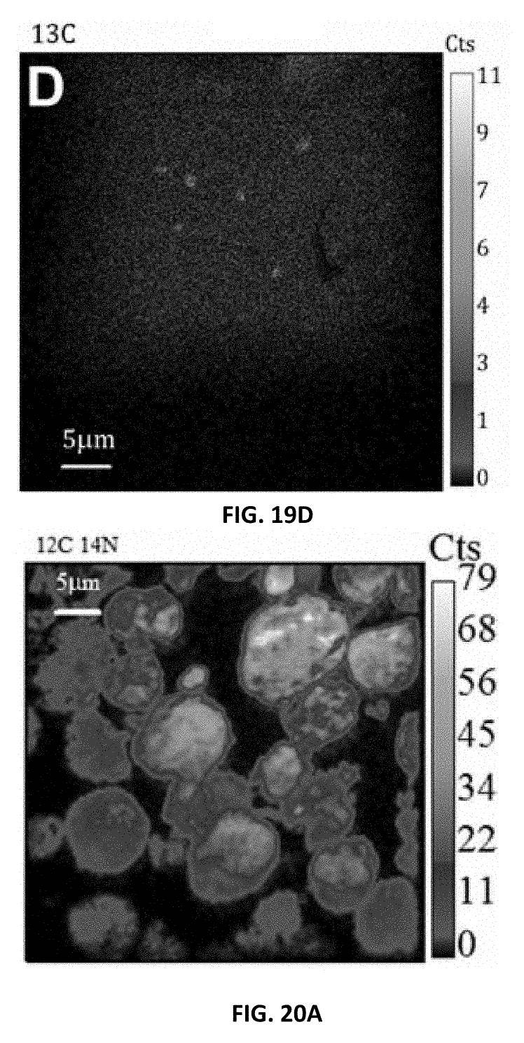

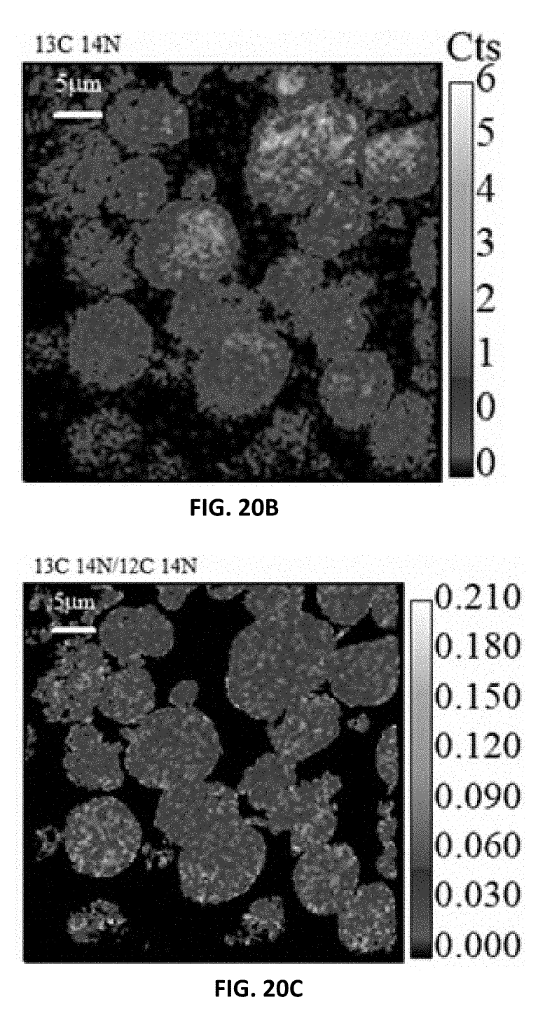

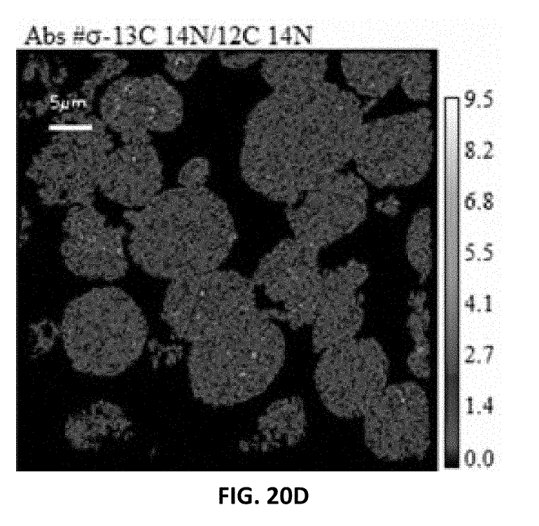

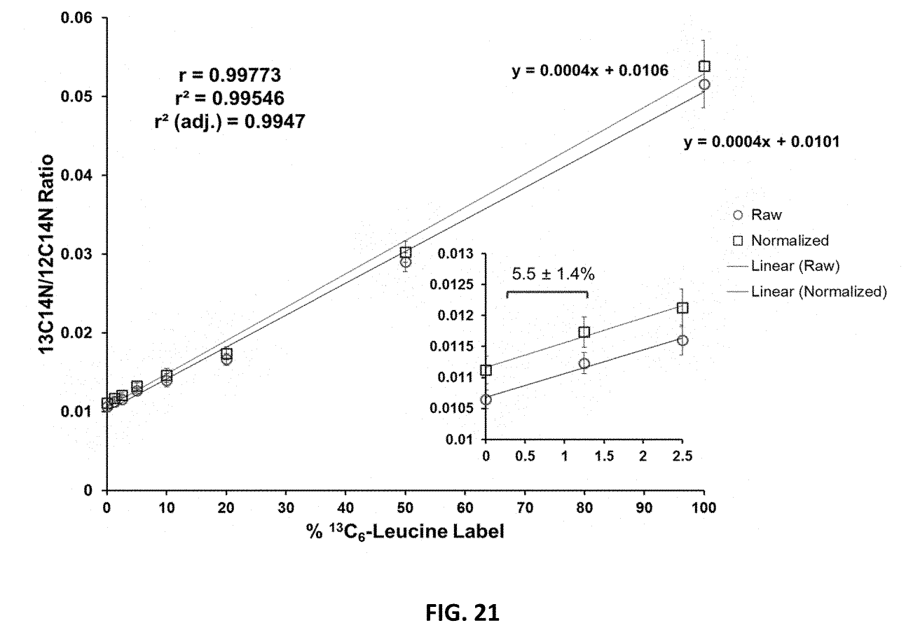

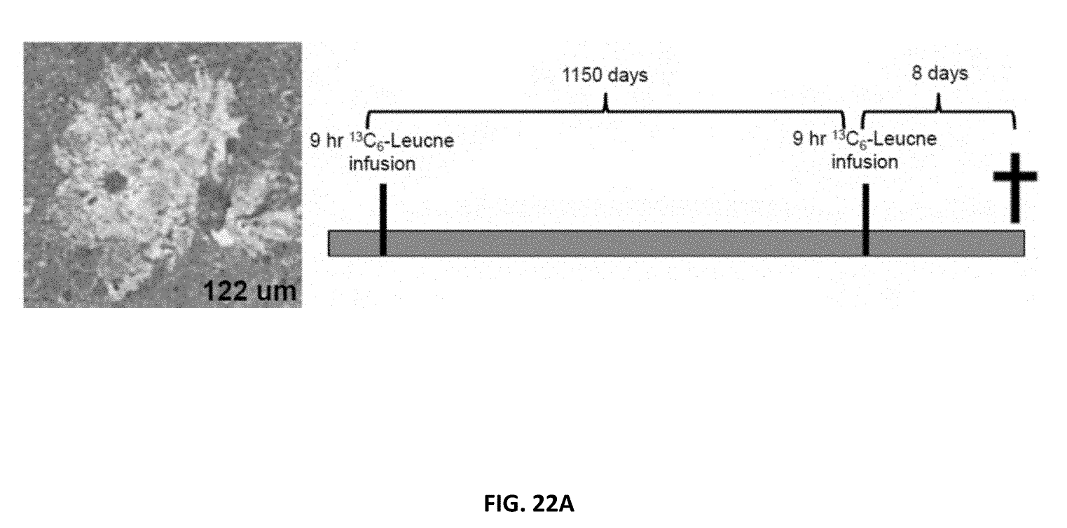

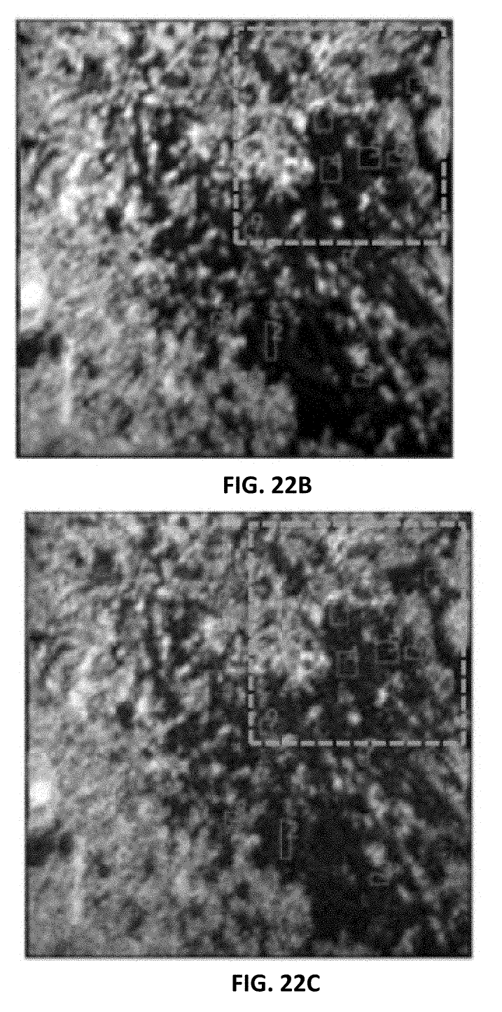

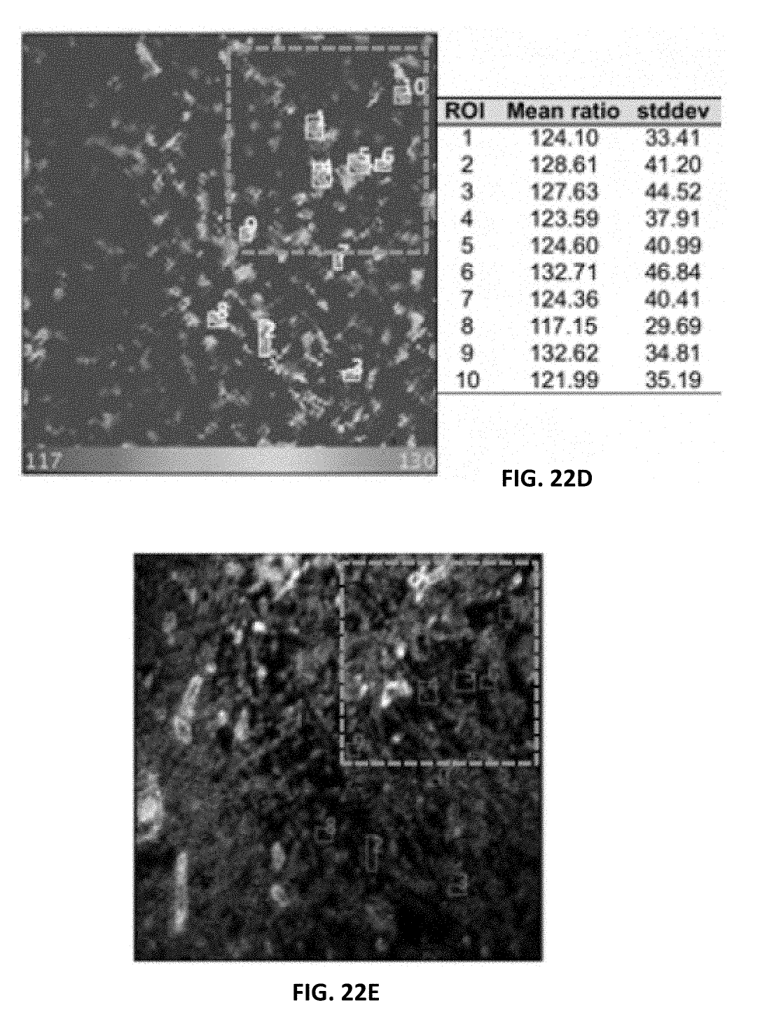

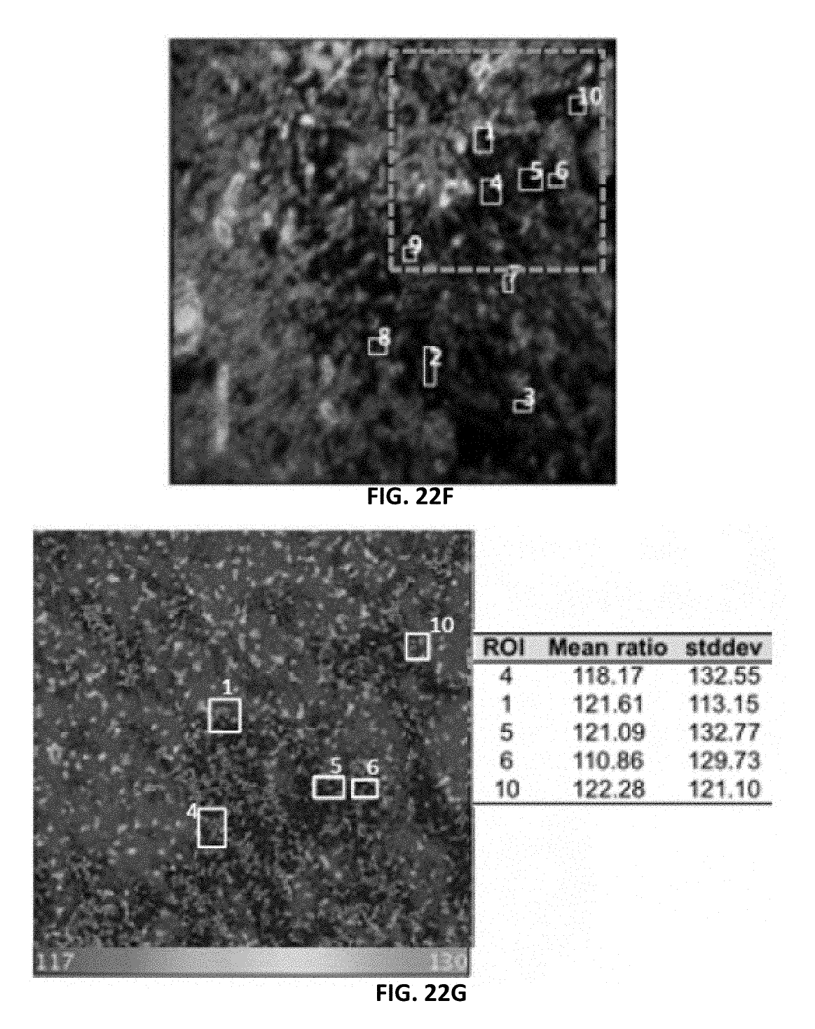

Improved morphology and topology was discovered when imaging carbon as a cyanide ion (see e.g., FIG. 19). A NanoSIMS standard curve was determined (see e.g., FIG. 20 and FIG. 21). Alzheimer's Disease Participant data is shown in FIG. 22.

The data showed that (i) imaging carbon as a CN⁻ molecule yields more visually informative data; (ii) the capability to detect increased ¹³C/¹²C ratios with only 1.25% labeling of cells for 7 days; and (iii) the first detection and calculation of carbon ratios human AD brain.

These experiments can allow for kinetic modeling of Aβ plaque deposition with ¹³C/¹²C ratios and area in addition to known labeling infusion and time and applications to other proteinopathies and biomolecules are feasible.

### Example 3: Stable Isotope Labeling & Quantitative Mass Spectrometry Imaging of Aβ Plaque Deposition in Human Ad

The following example shows the first direct measurements of the rates of amyloid plaque growth in the human brain can be measured by Stable Isotope Labeling Kinetics (SILK), and that amyloid plaque deposition can be quantified to address the unanswered question of “What is the rate of amyloid Alzheimer's pathology in humans”.

We will utilize prior brain donations from volunteers that were labeled with the stable isotope ¹³C₆-leucine during life, later died of natural causes, and donated their brain for research at the Washington University Alzheimer's Disease Research Center (ADRC). These brain donations contain proteins, which were synthesized during the labeling period, and can now be measured in ultra-high (100 nm) resolution to quantify new protein generation and clearance in the human brain. As most of the brain samples have Alzheimer's disease pathology, we will be able to image how this pathology changes over time. We will use NanoSIMS isotopic imaging of ultrathin sections of post-autopsy AD brain tissue, which is labeled during life, to detect the newly generated proteins in the human brain and around amyloid plaques. With this technique, we are able to: i) determine the ¹³C/¹²C ratio, ii) have a high signal-to-noise ratio and iii) image micro-ultrastructural features of the plaque and surrounding neural and glial features. We expect the NanoSIMS can successfully produce carbon isotope maps in brains from AD patients; newly synthesized protein, inferred from the ¹³C₆-leucine label, will be detected in the surrounding plaque area. Data will be generated from seven brain donations, which were previously labeled with SILK at early disease stages (pre-symptomatic to mild). These data will allow the visualization of amyloid plaque growth in vivo and will be central to accurately computing Aβ brain kinetics in a computational mathematical framework.

Using data from NanoSIMS analysis of carbon isotopes—individual isotope signal intensities, ¹³C/¹²C ratios, and areas (nm²) of ¹³C (i.e., newly synthesized protein)—we will enhance our mathematical model to quantify newly synthesized protein deposition into plaques factoring in the period between labeling and time of death. We expect to successfully implement a computational mathematical model of Aβ deposition, as was previously achieved for plasma and CSF. These calculations are central to understanding the AD pathophysiology process to better develop tests of AD pathology and also to estimate the dose and frequency of drugs, which are now targeting AD pathology.

Alzheimer's disease (AD) is a devastating neurodegenerative disease characterized by progressive cognitive decline. The disease progression is irreversible; no therapeutics can prevent, slow, or cure AD. Its impact on human health is significant. AD affects an estimated 5.3 million individuals in the USA (1). This number is projected to increase to 13.8 million by 2050—representing a true epidemic—and cost more than $1 trillion to our national healthcare system (1, 2). One prominent feature of AD is a marked thousand-fold increase in extracellular amyloid-beta (Aβ), implicated as a toxic neurodegeneration-inducing species (3). The accumulation of Aβ leads to one of the pathological hallmarks of AD—amyloid plaques.

Recent studies utilizing in vivo incorporation of the stable isotope ¹³C₆-leucine into Aβ in AD patients (4, 5) demonstrated that while the production of Aβ40 and 42 are similar between AD and control (non-AD, age-matched) subjects, the clearance of Aβ is decreased by approximately 30-50% (6, 7). With increasing age, the single largest risk factor of AD, the half-life of Aβ (i.e., turnover or clearance), slows 2.5-fold from 3.8 hr to 9.4 hr (6, 7). Intriguingly, with the onset of amyloidosis, only the Aβ42 isoform demonstrated faster turnover kinetics, possibly due to a rapid deposition into plaques for ˜50% of all Aβ42 produced (7), suggested by a positive correlation between increased Aβ42 turnover kinetics and rate of fibrillar amyloidosis as measured by PET Pittsburgh compound B (PIB) (7). This is consistent with studies that implicate Aβ42 as the major constituent in amyloid plaques. Despite advances in our understanding of Aβ kinetics in vivo, there is a critical gap in our knowledge of AD amyloid pathology the brain. First, these studies (7) relied on CSF and plasma, which are indirect measures of brain compartment Aβ. Next, there is evidence demonstrating that PIB binds to only a subset of Aβ from plaques enriched from AD brain (8). The implication of these recent findings suggest that studies utilizing PIB or amyloid imaging agents to measure Aβ in patients for early detection and monitoring of AD may be potentially underestimating the extent and rate of Aβ deposition and/or only measuring localized fibrillar pathology. Correct measures are critical for drug trial programs, which need to estimate the amount and rate of amyloid to estimate dose and frequency.

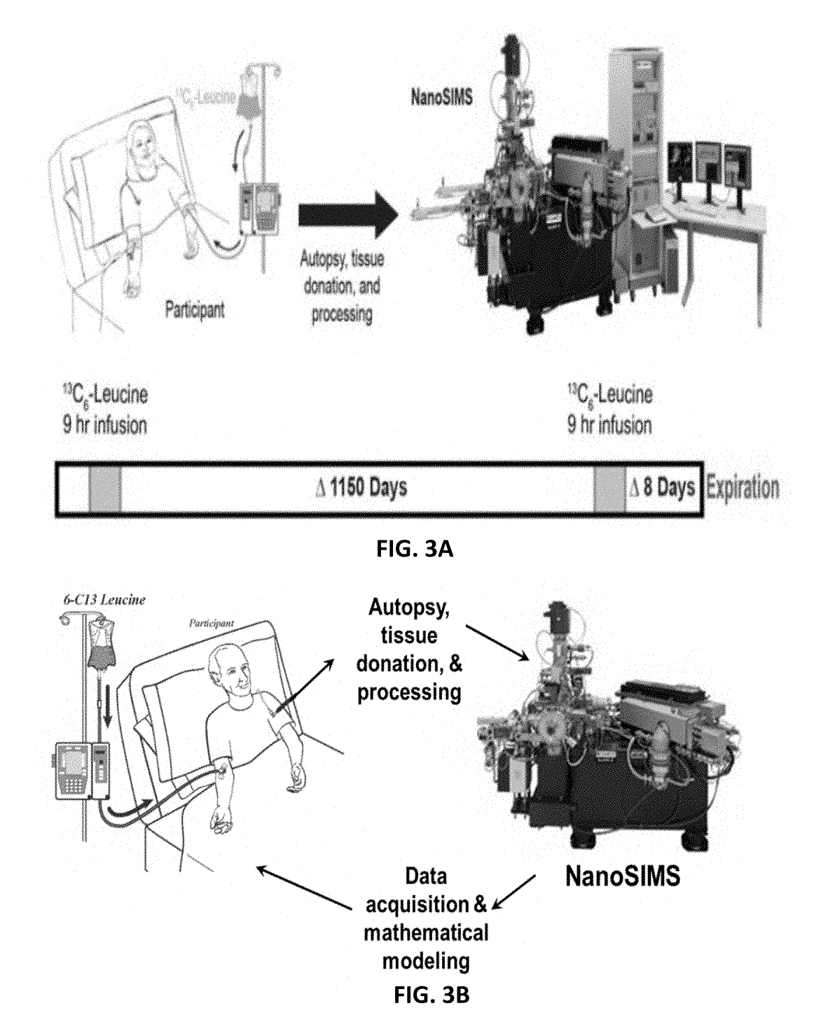

Thus, we hypothesize that: i) direct measurement of the rates of amyloid pathology growth is possible by SILK brain studies in humans, ii) the irreversible loss of Aβ is largely due to plaque deposition, and iii) that the rate of this deposition may be greater and occurs earlier than previously reported by PET PIB studies. This translationally-focused proposal couples in vivo brain SILK with nanoscale Secondary Ion Mass Spectroscopy (NanoSIMS) imaging to spatially and quantitatively profile deposition of newly synthesized protein into amyloid plaques in AD brain (FIG. 3). The proposed study provides a unique, first-in-human, opportunity to measure Aβ kinetics directly in the brain with AD pathology. The outcomes of these objectives will provide new insights in order to develop improved diagnostic test(s) to detect the early, prodromal stage of AD and to better understand the AD amyloid pathology process, in order to accelerate AD drug development.

NanoSIMS is an advanced mass spectrometry imaging technology that allows for the generation of nanoscale isotopic maps through the parallel acquisition of up to five isotopes at the subcellular level, with high sensitivity (≤1 ppm) and high spatial resolution (50-100 nm) (11). Yet, this technology has been rarely utilized in the Biological and Biomedical Sciences. While NanoSIMS has recently been applied to the study of AD (12, 13), NanoSIMS imaging has not been used for protein quantitation or in combination with SILK for the measurement of in vivo protein kinetics in normal or diseased brain (e.g. AD). We propose to characterize the spatial distribution of newly synthesized protein (via ¹³C₆-leucine labeling) deposition into amyloid plaques in AD brain tissue, while capitalizing on the temporal component (i.e., varying times between labeling and time of death in our cohort) to calculate deposition kinetics.

As a result, this research could shift the current paradigm by providing the first quantitative measure of deposition into amyloid plaques by exploiting cutting-edge methodologies never before leveraged in the field of AD or neurodegeneration. These measures will lead to a more accurate estimate of plaque growth, which can be utilized to determine the rate of Aβ pathology prior to the onset of AD clinical symptoms. The full implications and extent of this work will facilitate a direct comparison between depositions determined by our SILK-NanoSIMS imaging approach to those derived from PET-PIB.

### (I) Acquire In Vivo Stable Isotope Labeled Images of Aβ Plaques in Human AD Brain Utilizing a Validated NanoSIMS Imaging Protocol.

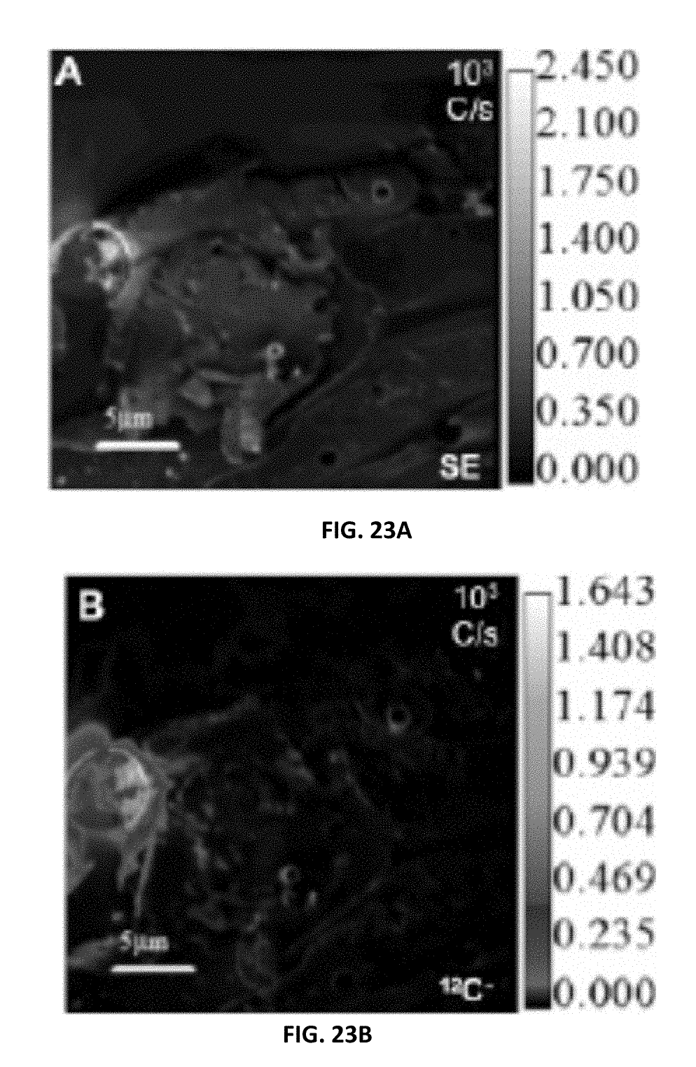

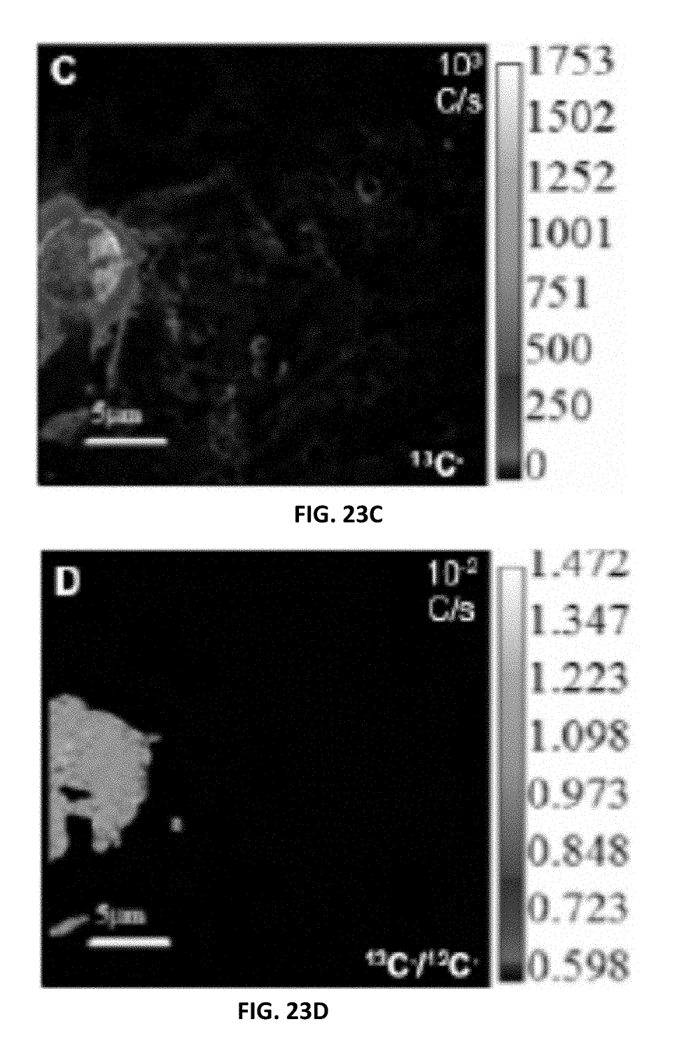

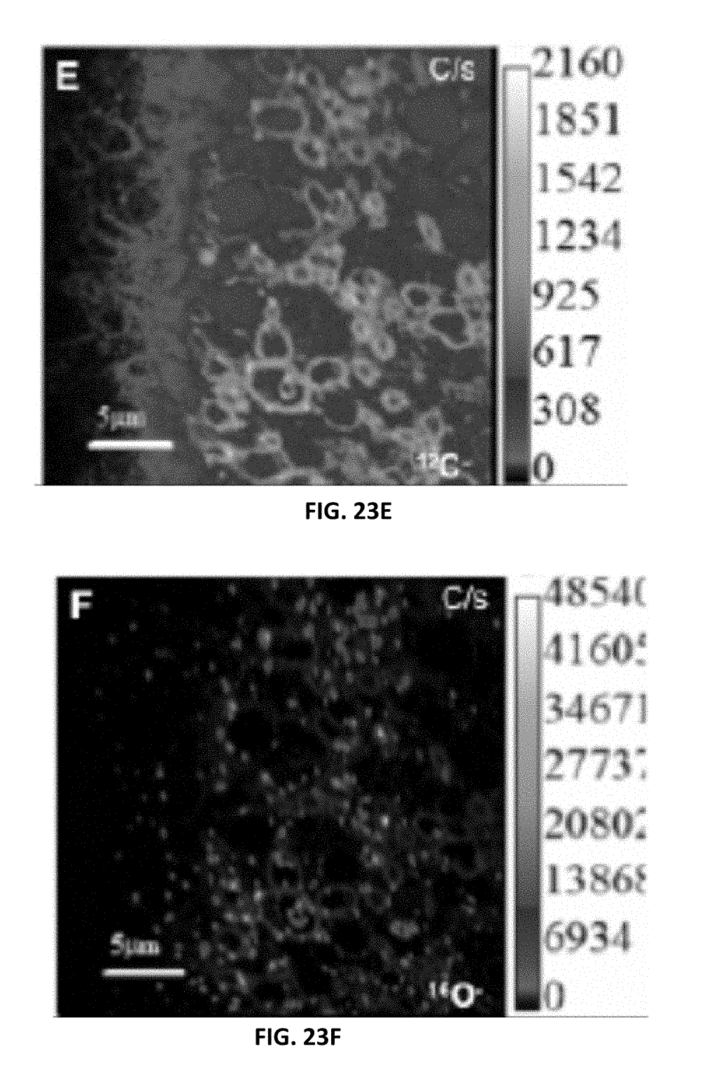

NanoSIMS is an important and routinely employed analytical method for investigating isotopic compositions in the fields of Material Science, Cosmochemistry, and Geochemistry; however, this technique remains under-utilized in the Biological and Biomedical Sciences. Unlabeled brain still contains a natural abundance ¹³C (1.1% of the abundance of ¹²C) that is detectable and measurable by the NanoSIMS (FIG. 23). In this technique, a focused (˜100 nm in diameter) Cs⁺ primary ion beam is electrostatically scanned across the defined region of interest (ROI) in the tissue (i.e., plaque) producing secondary ions. These secondary ions are transmitted through ion optics (similarly to visible light in microscopes, but using electrostatic lenses) for mass separation and detection of ¹²C and ¹³C (monoisotopic mass 12.0 and 13.0 Da, respectively) for the generation of a quantitative spatial profile of carbon isotopes in the ROI. ¹³C₆-leucine labeled autopsied AD brain tissue obtained from the ADRC, which has been formalin fixed, will be embedded in Epon resin (EM core; Dr. Robert Schmidt). Thin sections (˜0.5 μm thick) will be produced with an ultra-microtome and stained with toluidine blue for optical microscopy. Adjacent semi-thin sections (300-400 nm thick) will be directly deposited on high-purity silicon wafers for NanoSIMS analysis (FIG. 3). Images of the tissue will be taken with a scanning electron microscope (SEM), in order to document plaque locations and provide an absolute coordinate system for the tissue. For samples needing to be analyzed in the SEM, we will first gold coat to prevent any carbon deposition that is typical for SEM. In this way, we can use already developed coordinate transformation software, which uses mathematical algorithms to translate those ROIs found in the SEM, to re-locate the same ROIs in the NanoSIMS instrument. Multiple patient samples can be placed on a single Si wafer for analysis in a single run, optimizing efficiency and minimizing cost. After image acquisition, we will determine quantitative mass images of ¹²C and ¹³C and calculate the ¹³C/¹²C ratios of the sample with already developed, well-established image processing software—both OpenMIMS and L'Image. With the high purity of the ¹³C label, we expect to be able to observe at least 1-100s of permil (i.e., the percent difference from atomic abundance multiplied by 10) enrichment in ¹³C. The SEM images, taken beforehand, will allow coordinated analysis of ultra-structural features of the plaque and surrounding neural and glial features (akin to MRI spatial enhancement of PET scans), due to its higher lateral resolution (˜5 down to 0.4 nm with feSEM) than that of the NanoSIMS. These combined techniques will also allow us to more precisely define the spatial areas of ¹³C/¹²C enrichment analyzed by the NanoSIMS (akin to PET), in order to provide ¹³C enrichment density per given area (nm²) vs non-enrichment and to facilitate direct comparison between intrinsic tissue structures.

With the successful completion of establishing an appropriate NanoSIMS imaging protocol, data will be generated from seven brain donations which were previously labeled with SILK (TABLE 7). A minimum of 10-15 plaques per patient sample (analytical replicates) in the frontal lobe will be measured; ¹³C above its natural abundance, calculated by the ¹³C/¹²C ratio in the ROI (i.e., plaque), will indicate the presence of new protein deposition. The ¹³C/¹²C ratio, its spatial distribution (enhanced by complementary high-resolution SEM imaging), and the temporal component (labeling to time of death) will all generate critical values for a computational mathematical framework of Aβ plaque deposition.

TABLE 7
Patient cohort for NanoSIMS imaging of Aβ plaques

| | Amyloid | | | | | Δ between |
|---|---|---|---|---|---|---|
| | Status | | | | | labeling and |
| Participant | PET-PiB | CDR^{(a)} | AD Dementia | MMSE^{(a)} | Gender | DOE (Days) |
| 1 | pos | 2 | Moderate | 21 | M | 1568 |
| 2 | pos | 1 | Mild | 24 | M | 2527 |
| 3 | pos | 1 | Mild | 27 | M | 876 |
| 4 | pos | 0.5 | Questionable | 25 | F | 1119 |
| 5 | neg | 0.5 | Questionable | 30 | F | 1183 |
| 6 | pos | 1 | Mild | 11 | M | 1648 |
| 7 | pos | 1 | Mild | 22 | M | 1150 and 8^{(b)} |

CDR, Clinical Dementia rating;
MMSE Mini Mental State Exam;
DOE Date of Expiration
^{(a)}CDR and MMSE as most recent
^{(b)}This participant was in two SILK studies and thus has two time lapses

(II) Develop a Computational Mathematical Framework that Accurately Quantifies Leucine-Labeled Protein Deposition into Plaques.

We have previously developed a physiologically relevant multi-compartmental model (7, 9, 19), to assess Aβ kinetics in vivo and the relationship between Aβ isoform kinetics and patient demographics, such as, age, Clinical Dementia Rating (CDR), and amyloid status. Within this model, we define the variable of the irreversible loss of Aβ (v38, v40, v42), which includes deposition, degradation (enzymatic or via proteosome), and transport across the blood-brain barrier (19). Currently, measuring Aβ isoforms in plasma to solve a component of Aβ irreversible loss through the blood-brain barrier in this model is being studied. However, until now, determining irreversible loss due to plaque deposition in vivo and with methods alternative to PET-PIB (8) has been a missing link.

Thus, we propose to use the ¹³C/¹²C ratio densities in “hotspots”—¹³C enrichment beyond its natural abundance—in imaged plaques per area (nm²) versus the ¹³C/¹²C ratio densities in “non-hotspot” areas (i.e., the remainder of the plaque). We predict proteins newly synthesized and deposited within the 9 hr ¹³C₆-leucine injection period to be labeled, whereas unlabeled protein would represent deposition before and after the SILK study. This distinction is aided by the fact that plaques demonstrate lateral circumferential growth (17). The bulk composition of the plaque would represent deposition prior to labeling, newly synthesized protein deposition would theoretically demonstrate a ring of ¹³C enrichment around the plaque core, and, finally, newly deposited protein after labeling but before death would be represented by a ring of ¹²C enrichment around the periphery of the ¹³C abundant ring (FIG. 24). Further, we do not expect significant degradation of proteins after deposition into plaques, thus preserving ¹³C₆-leucine labeling. From the ¹³C/¹²C ratios per area (hotspots vs non-hotspots) across a cohort of patients with varying times between labeling and death (TABLE 7), we can derive total protein plaque deposition rates in AD.

Techniques such as immunogold labeling with SEM or TEM, in parallel with NanoSIMS imaging and/or the development of immuno-depletion of Aβ from the plaques followed by NanoSIMS, would be potentially able to parse Aβ deposition from total protein deposition and are contemplated. Additionally, development of methods to isolate Aβ from plaques within tissue sections for biochemical measurements of 13C/12C will provide orthogonal validation of NanoSIMS measurements. Ultimately, this approach of quantifying nanometer resolution images of SILK labeled proteins in the human brain can be easily translated to a variety of neurodegenerative diseases including Parkinson's disease, amyotrophic lateral sclerosis, and multiple sclerosis, and stroke, as basic physiology as basic physiology of human brain biomolecule kinetics.

### Example 3 References

1. (2010-2050) estimated using the 2010 census. Neurology 80, 1778-1783
2. (2015) Changing the Trajectory of Alzheimer's Disease: How a Treatment by 2025 Saves Lives and Dollars
3. Hardy, J. A., and Higgins, G. A. (1992) Alzheimer's disease: the amyloid cascade hypothesis. Science 256, 184-185
4. Mawuenyega, K. G., Kasten, T., Sigurdson, W., and Bateman, R. J. (2013) Amyloid-beta isoform metabolism quantitation by stable isotope-labeled kinetics. Anal Biochem 440, 56-62
5. Bateman, R. J., Munsell, L. Y., Morris, J. C., Swarm, R., Yarasheski, K. E., and Holtzman, D. M. (2006) Human amyloid-beta synthesis and clearance rates as measured in cerebrospinal fluid in vivo. Nat Med 12, 856-861
6. Mawuenyega, K. G., Sigurdson, W., Ovod, V., Munsell, L., Kasten, T., Morris, J. C., Yarasheski, K. E., and Bateman, R. J. (2010) Decreased clearance of CNS beta-amyloid in Alzheimer's disease. Science 330, 1774
7. Patterson, B. W., Elbert, D. L., Mawuenyega, K. G., Kasten, T., Ovod, V., Ma, S., Xiong, C., Chott, R., Yarasheski, K., Sigurdson, W., Zhang, L., Goate, A., Benzinger, T., Morris, J. C., Holtzman, D., and Bateman, R. J. (2015) Age and amyloid effects on human central nervous system amyloid-beta kinetics. Ann Neurol
8. Matveev, S. V., Spielmann, H. P., Metts, B. M., Chen, J., Onono, F., Zhu, H., Scheff, S. W., Walker, L. C., and LeVine, H. (2014) A distinct subfraction of Aβ is responsible for the high-affinity Pittsburgh compound B-binding site in Alzheimer's disease brain. J Neurochem 131, 356-368
9. Potter, R., Patterson, B. W., Elbert, D. L., Ovod, V., Kasten, T., Sigurdson, W., Mawuenyega, K., Blazey, T., Goate, A., Chott, R., Yarasheski, K. E., Holtzman, D. M., Morris, J. C., Benzinger, T. L., and Bateman, R. J. (2013) Increased in vivo amyloid-β42 production, exchange, and loss in presenilin mutation carriers. Sci Transl Med 5, 189ra177
10. Roberts, K. F., Elbert, D. L., Kasten, T. P., Patterson, B. W., Sigurdson, W. C., Connors, R. E., Ovod, V., Munsell, L. Y., Mawuenyega, K. G., Miller-Thomas, M. M., Moran, C. J., Cross, D. T., Derdeyn, C. P., and Bateman, R. J. (2014) Amyloid-β efflux from the central nervous system into the plasma. Ann Neurol 76, 837-844
11. Steinhauser, M. L., and Lechene, C. P. (2013) Quantitative imaging of subcellular metabolism with stable isotopes and multi-isotope imaging mass spectrometry. Semin Cell Dev Biol 24, 661-667
12. Quintana, C., Bellefqih, S., Laval, J. Y., Guerquin-Kern, J. L., Wu, T. D., Avila, J., Ferrer, I., Arranz, R., and Patiho, C. (2006) Study of the localization of iron, ferritin, and hemosiderin in Alzheimer's disease hippocampus by analytical microscopy at the subcellular level. J Struct Biol 153, 42-54
13. Quintana, C., Wu, T. D., Delatour, B., Dhenain, M., Guerquin-Kern, J. L., and Croisy, A. (2007) Morphological and chemical studies of pathological human and mice brain at the subcellular level: correlation between light, electron, and nanosims microscopies. Microsc Res Tech 70, 281-295
14. Steinhauser, M. L., Bailey, A. P., Senyo, S. E., Guillermier, C., Perlstein, T. S., Gould, A. P., Lee, R. T., and Lechene, C. P. (2012) Multi-isotope imaging mass spectrometry quantifies stem cell division and metabolism. Nature 481, 516-519
15. Kern, J. L., Lechene, C. P., and Lee, R. T. (2013) Mammalian heart renewal by pre-existing cardiomyocytes. Nature 493, 433-436
16. Wildburger, N. C., Wood, P. L., Gumin, J., Lichti, C. F., Emmett, M. R., Lang, F. F., and Nilsson, C. L. (2015) ESI-MS/MS and MALDI-IMS Localization Reveal Alterations in Phosphatidic Acid, Diacylglycerol, and DHA in Glioma Stem Cell Xenografts. J Proteome Res 14, 2511-2519
17. Yan, P., Bero, A. W., Cirrito, J. R., Xiao, Q., Hu, X., Wang, Y., Gonzales, E., Holtzman, D. M., and Lee, J. M. (2009) Characterizing the appearance and growth of amyloid plaques in APP/PS1 mice. J Neurosci 29, 10706-10714
18. Engler, H., Forsberg, A., Almkvist, O., Blomquist, G., Larsson, E., Savitcheva, I., Wall, A., Ringheim, A., Langström, B., and Nordberg, A. (2006) Two-year follow-up of amyloid deposition in patients with Alzheimer's disease. Brain 129, 2856-2866
19. Elbert, D. L., Patterson, B. W., and Bateman, R. J. (2015) Analysis of a compartmental model of amyloid beta production, irreversible loss and exchange in humans. Math Biosci 261, 48-61

### Example 4

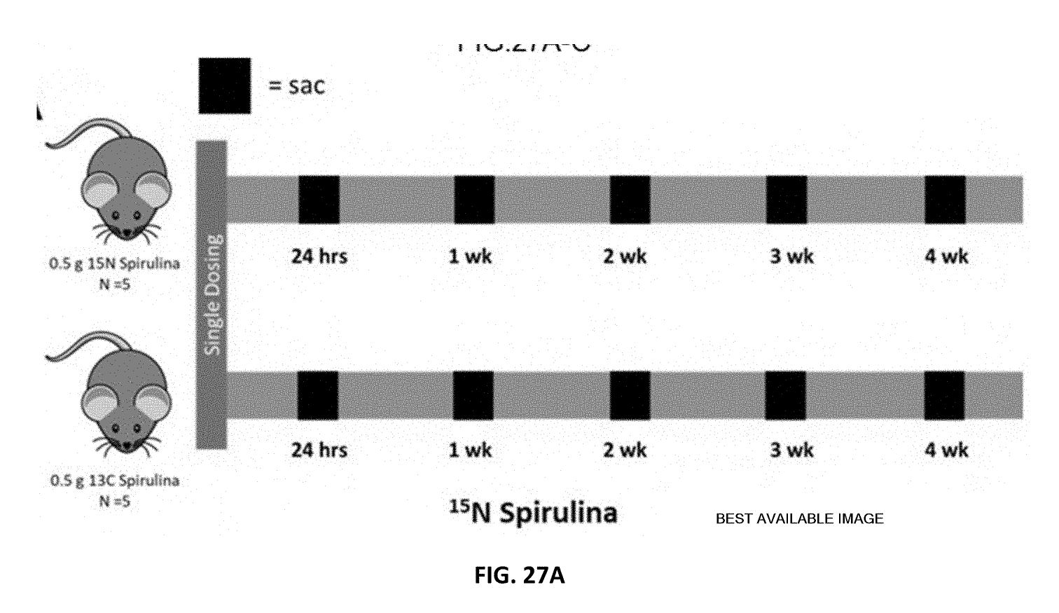

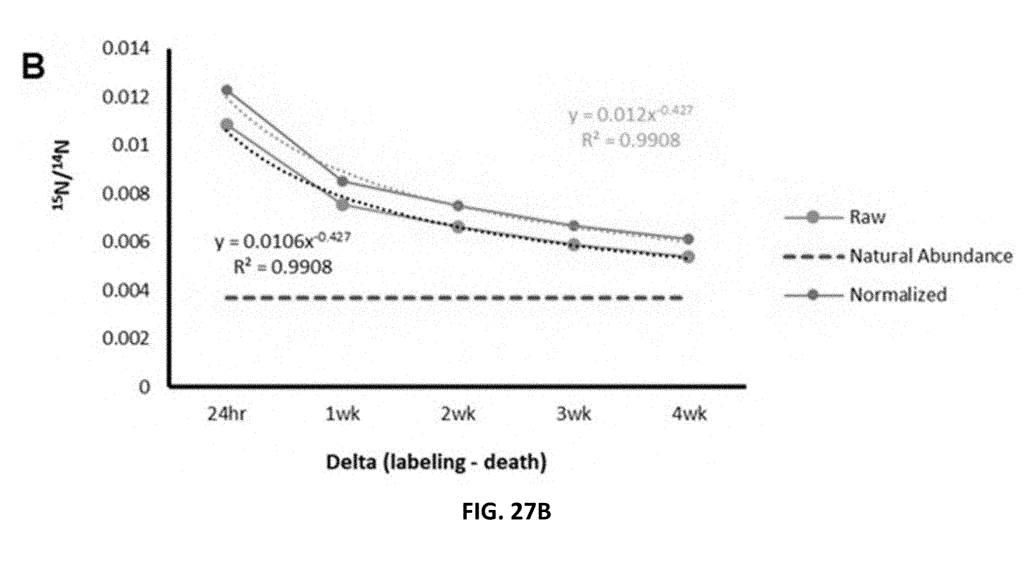

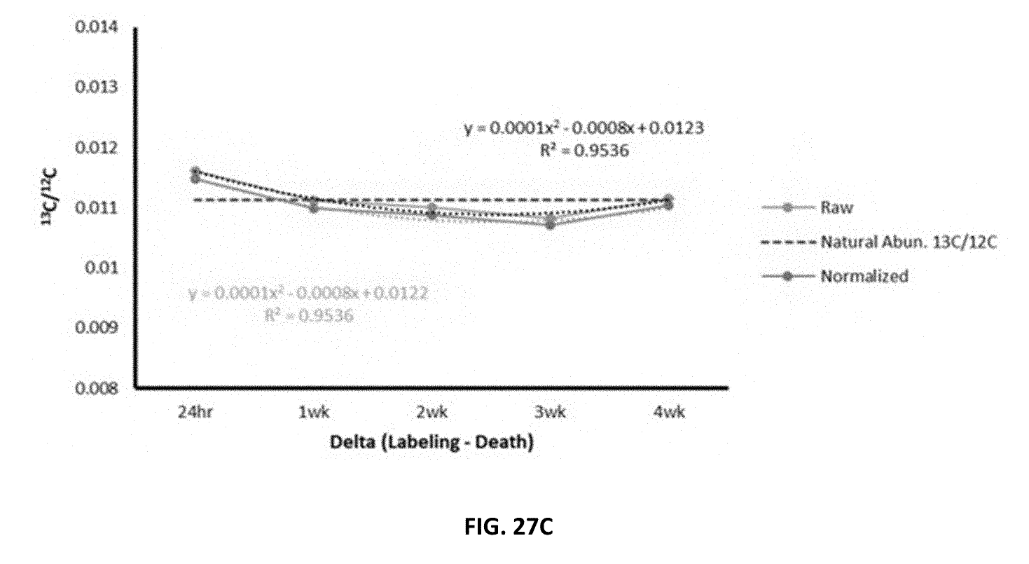

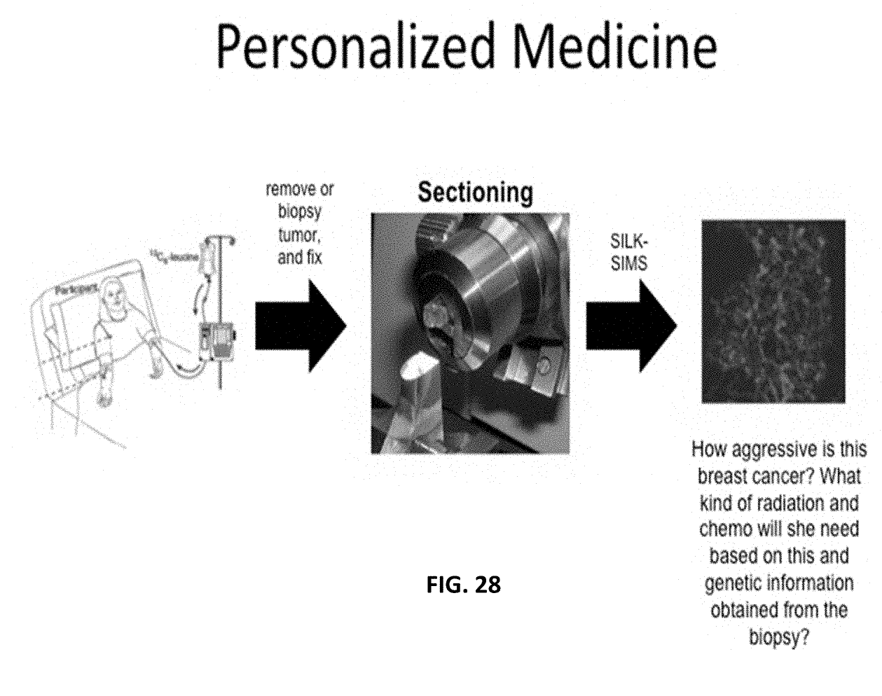

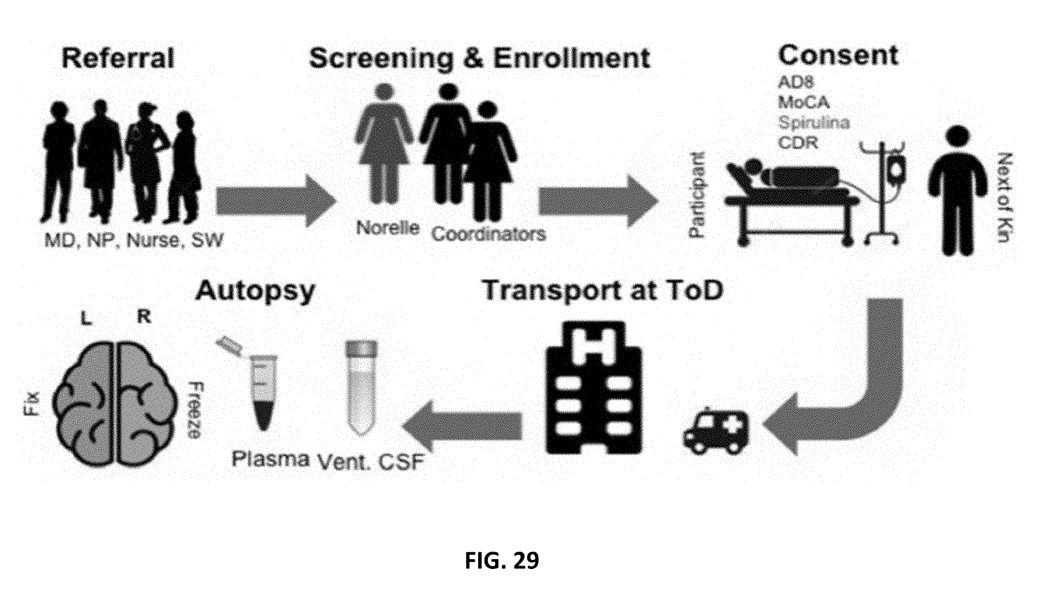

In the proof-of-principle study, I was able to detect ¹³C label in brain tissue of this patient who passed 8 days after labeling (FIG. 3 in ref. Wildburger et al., 2018; PMID 29623063), but was unable to detect ¹³C in other participants who died months to years after labeling (n=5; 0.5-4 years between study and death; data not shown). This is attributed to tracer washout and the high isotopic background of carbon (¹³C has a natural abundance of 1.1%; there are 90 ¹²C atoms for every ¹³C atom). Although low levels of ¹³C can be quantified with SILK-SIMS, the instrument sampling time is ˜24 hours/plaque to obtain sufficient signal (i.e., secondary ions). This data acquisition rate is not suitable even for small cohort studies. To optimize the acquisition time, we tested two labeling protocols in 6-month-old male APP/PS1 mice treated with commercially available ¹⁵N— or ¹³C-labeled Spirulina from Cambridge Isotope Laboratories (FIG. 27). Spirulina an edible powder of blue-green algae, and is considered a dietary supplement. I obtained a designated lot of Spirulina and tested it for heavy metals and microcystin toxins. Mice received a single oral dose (0.5 grams) of either ¹⁵N-Spirulina or ¹³C-Spirulina (FIG. 27A), in their water (ad libitum) and were returned to normal drinking water 12 hours later. Subsequently, the treated mice were sacrificed at specific time intervals. SILK-SIMS imaging revealed that ¹⁵N was enriched in brain tissue at 4 weeks after the single oral dose (FIG. 27B). By contrast, ¹³C label rapidly diminished at 24 hours after labeling. Therefore, ¹⁵N has substantially longer half-life in brain tissue than ¹³C. The use of ¹⁵N-Spirulina has the following advantages: (i) it is inexpensive (ii) the increased heavy isotope signal reduces SILK-SIMS data acquisition times and background error; (iii) the ¹⁵N signal is not affected by the use of carbon-rich embedding resin in fixed specimens, which can affect the ¹³C signal by reducing the measured ¹³C levels below natural abundance (FIG. 27C); and (iv)¹⁵N has a natural abundance of 0.37% (for every ¹⁵N atom there are 272 ¹⁴N atoms), which generates less background noise then for ¹³C measurements. Due to the success of ¹⁵N-Spirulina labeling in mice, I proceeded to use 10 grams of ¹⁵N-Spirulina from the designated lot in the hospice pilot study (Study Protocol FIG. 29).

We obtain fully informed consent or assent with proxy consent. Then, we administer universally labeled ¹⁵N-spirulina to the enrolled hospice participants. The participants' cognitive function and dementia severity is assessed with the Montreal Cognitive Assessment (MoCA)⁴⁶ and AD8^{47,48} screening tools. We perform a retrospective interview of a reliable collateral source to assess cognitive change using the clinical dementia rating (CDR)^{49,50} scale. At death, participants are taken to autopsy and a full National Institute on Aging-Alzheimer's Association (NIA-AA) pathological workup^{16,17} is performed. The autopsy and workup will allow confirmation of AD diagnosis, define amyloid pathology in cognitively normal participants (CDR0+), and provide linkage between human autopsy specimens and clinical and laboratory data.

The study will generate a histological map of in situ ¹⁵N incorporation.³⁷⁻³⁹ Incorporation of the ¹⁵N is detected by an increase in the ¹⁵N/¹⁴N ratio relative to an unlabeled participant. The entorhinal cortex, hippocampus, and cortical ribbons of parietal and frontal lobes⁵¹ are sampled. These regions are embedded in LR White resin, and thin sections are produced as described previously¹⁰ for light microscopy, scanning electron microscopy (SEM), and SILK-SIMS imaging.

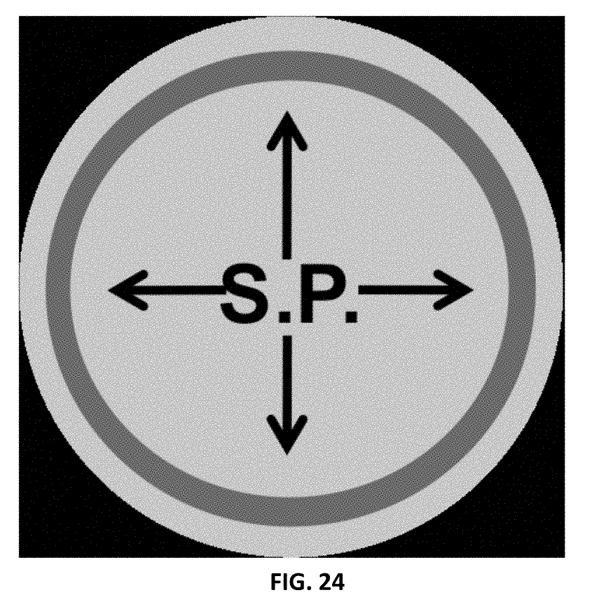

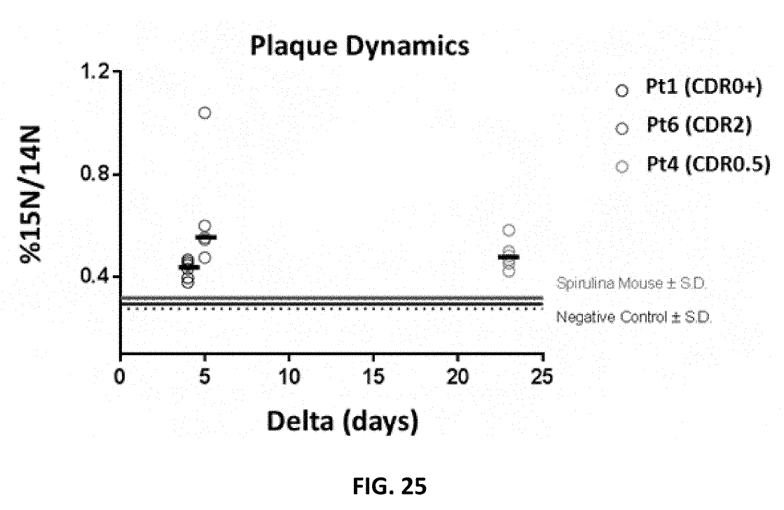

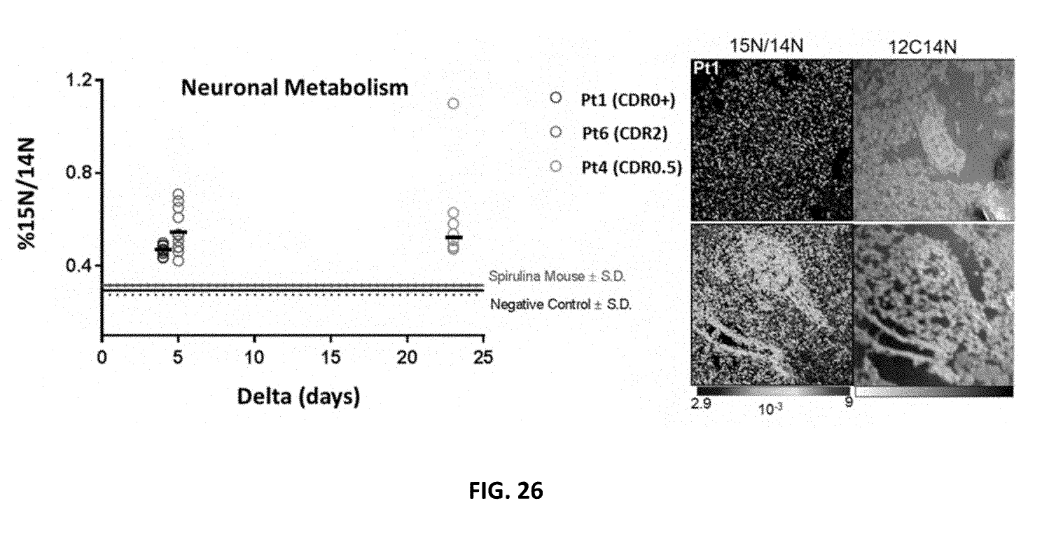

Utilizing this approach, I quantified ¹⁵N incorporation into plaques in SILK-SIMS images.¹⁰ The ¹⁵N/¹⁴N ratios were measured with SILK-SIMS in the first 3 patients of our cross-sectional patient cohort with varying times between labeling and death (Delta). These data are being used to determine plaque dynamics³ as a function of disease severity relative to age-matched controls (FIG. 25) as well as neuronal metabolism (via the incorporation of the ¹⁵N tracer) (FIG. 26).

### Example 5: Silk-SIMS for Cancer Applications

### (I) Visualizing Payload Delivery.

Visualizing payload delivery can be divided into two categories. First, nanoparticles packaged with drugs. A fundamental problem in the field of nanoparticle technology is imaging them without causing collapse, which requires specialized techniques to visualize. Further, visualization of nanoparticles and drug can occur simultaneously. Second, cell-based therapies such as using bone marrow-derived mesenchymal stem cells packaged with an oncolytic virus are contemplated. In particular, isotope labeled modified or engineered viruses.

We plan to SILK-SIMS to visualize nanoparticle and cell-based therapies for payload delivery. We will have Cisplatin or other drugs, or oncolytic viruses produced with a stable isotope label (e.g., 15-Nitrogen, Deuterium, ¹⁸O, ¹⁷O) or halogeneous (¹⁹F and ⁸¹Br) tracer incorporated into its chemical structure. Alternatively in the case of Cisplatin and other drugs we can monitor the heavy metal that is already a component of its chemical structure. These compounds and biological agents can be packaged into nanoparticles or bone marrow-derived mesenchymal stem cells. The delivery vehicles will be labeled on their surface with a tracer that is different from that which labels the compound or virus. The nanoparticles can be delivered to cells, animals, or humans while the mesenchymal stem cells can be delivered to animals and humans via carotid artery injection or other means.

Harvested cells, and tissue biopsies or resections from human and animals will occur at pre-specified time points and be fixed and processed for SILK-SIMS analysis as previously described in Wildburger, N.C., et al. Amyloid-beta Plaques in Clinical Alzheimer's Disease Brain Incorporate Stable Isotope Tracer In Vivo and Exhibit Nanoscale Heterogeneity. Front Neurol 9, 169 (2018), herein incorporated by reference in its entirety. For cells there is one exception to this in the case of nanoparticle-mediated delivery. To examine nanoparticle-based drug delivery, cells will be grown on glass, aclar, or LX112 in culture before being embedded (with the glass etc) in resin. We will use hydrogen fluoride to dissolve the glass prior to ultra-thin sectioning as previously described. This novel technique allows us to see nanoparticles intact by SILK-SIMS imaging without risk of collapse of the particle during sample processing. Using the multiple detectors of the NanoSIMS instrument and the sub-cellular resolution (100 nm) we will be able to localize the distribution of the labeled nanoparticles and mesenchymal stem cells as well as the release (or lack thereof) their labeled payload.

### (II) Chemical Reactions.

Contemplated is the novel concept of using two labels on two different biomolecules and monitoring chemical and/or biological end products, which result in the concurrence to the two labels on a single biomolecule or chemical structure.

SILK-SIM can be used to image in time the chemical and/or biological end products of a reaction. For example, biomolecule A and biomolecule B would each be given two different stable isotope labels and administered as previously described herein. The co-localization of the two stable isotope labels, like co-localization of two channels in confocal microscopy would indicate concurrence of the two labels on a single biomolecule, which we hypothesize to be the synthetic end product of metabolism of A and B.

Cancer cells utilize predominantly glycolysis to produce energy and effect known as the Warburg effect. To monitor utilization of metabolic precursors in cancer the following can be performed. The first step in the glycolysis pathway: Glucose (2H)+ATP (15N)->Glucose-6-Phosphate. Where glucose is labeled with Deuterium (2H) and adenosine triphosphate (ATP) is labeled with Nitrogen 15 (¹⁵N). The co-localization of 2H and 15N will inform us about both the rate of production of glucose-6-phosphate, its localization at the cellular and sub-cellular level, as well as the rate of catabolism into the ultimate glycolysis end product—pyruvate.

These experiments would be performed on cancer cells lines and orthotopic animal models of cancer (glioma, breast, B-cell lymphoma etc). Labeled glucose and ATP would be delivered as previously described herein. Cells would be harvested and animals sacked and specified time points and fixed and processed for SILK-SIMS analysis as described herein.

Furthermore, utilizing the same methodology and models, the localization, metabolism, and chemical interactions of a one of more chemotherapy agents could be monitored in vivo provided each agent had its own unique stable isotope label.

### (III) Visualizing the Dynamics and Kinetics of Cancer Cells.

Stable isotope labeling has been primarily used in the context of biodistribution or drugs or metabolites, but has not been demonstrated in a kinetic context. For example, a single dose/bolus (IV or oral) of a stable isotope label can be given to a patient and multiple biopsies over time (e.g., breast cancer) can be performed. For example, the rate of glucose consumption in a given time interval with a given dose will inform oncologists as to the aggressiveness of the tumor. This in turn will aid in the design of a radio- and chemotherapy regime subsequent to full resection of the tumor.

15N-labeled glucose or glutamine will be delivered orally or by IV (8 hrs) to a patient with, for example, breast cancer. After the start of the infusion (or single oral dose), the patient will undergo 1-2 cm³ tissue punches every hour for 12 hours (the label infusion stops at 8 hrs). Biopsied samples over the course of the infusion and infusion cut off will be analyzed by SILK-SIMS to generate a histological map of in situ ¹⁵N-glucose or glutamine incorporation over time producing a real-time kinetic curve of cancer metabolism in a living patient. We will use these data to inform medical professionals as to the aggressiveness of the patient's tumor to better tailor medical treatment.

What is claimed is:

1. A system for measuring molecular flux of a biomolecule or therapeutic agent and determining the biomolecule or therapeutic agent location in a biological sample, wherein the biological sample is obtained from an individual to whom a composition comprising one or more stable isotope-labeled precursors or stable isotope-labeled therapeutic agents has been administered for a period of time sufficient for one or more isotope labeled precursors to become incorporated into a biomolecule of interest in the individual, the system comprising:
imaging the biological sample using Stable Isotope Labeling Kinetics (SILK) and nanoscale secondary ion mass spectrometry (NanoSIMS);
detecting spatially the biomolecule or isotope-labeled therapeutic agent in the biological sample; and
quantifying the molecular flux of the biomolecule or isotope-labeled therapeutic agent in the biological sample.

2. The system of claim 1, wherein at least two biological samples are obtained from the subject at different time points and analyzed by SILK-SIMS.

3. The system of claim 1, wherein the method or system comprises (i) electrostatically rastering a focused Cs+ primary ion beam across a defined region-of-interest (ROI) in the biological sample producing secondary ions used to measure the atomic composition of the biological sample surface; (ii) producing high-resolution quantitative image representing an in situ isotopic-histological map at 100 nm lateral resolution; (iii) acquiring one or more isotopes, optionally, in parallel; (iv) detecting and localizing a biomolecule or isotope-labeled therapeutic agent; or (v) quantitatively imaging the ratio of two stable isotopes of the same element.

4. The system of claim 1, wherein the system comprises determining the location of the biomolecule performed at a nanometer resolution, wherein the stable isotope precursor or stable isotope-labeled therapeutic agent comprises one or more of ²H, ¹³C, ¹⁵N, ¹⁸O, ¹⁷O, ³H, ¹⁴C, ³⁵S, ³²P, ¹²⁵I, ¹³¹I, ¹⁹F, ⁸¹Br ¹³C₆-leucine, ¹²C¹⁴N—, ¹³C¹⁴N—, and ¹⁶O—.

5. The system of claim 1, wherein the biological sample comprises: post-mortem tissue, tissue biopsies, cell culture, CSF, or brain tissue.

6. The system of claim 1, wherein a biomolecule is selected from the group consisting of lipids, proteins, peptides, or carbohydrates.

7. The system of claim 1, wherein the therapeutic agent is one or more of a conventional drug, a gene therapy construct, a chemotherapeutic agent, an antibiotics, a macromolecule, a protein bound drug, a cell-based therapies such as bone marrow-derived mesenchymal stem cells, an oncolytic virus, fractions of tissues or cells, a nanoparticles, a nucleic acid, a polypeptide, a siRNA, an antisense molecule, an aptamer, a ribozyme, a triple helix compound, an antibody, a small organic molecule or an inorganic molecule.

8. A method for measuring molecular flux of a biomolecule or therapeutic agent and determining the biomolecule or therapeutic agent location in a biological sample, wherein the biological sample is obtained from an individual to whom a composition comprising one or more stable isotope-labeled precursors or stable isotope-labeled therapeutic agents has been administered for a period of time sufficient for one or more isotope labeled precursors to become incorporated into a biomolecule of interest in the individual, the method comprising:
imaging the biological sample using Stable Isotope Labeling Kinetics (SILK) and nanoscale secondary ion mass spectrometry (NanoSIMS);
detecting spatially the biomolecule or isotope-labeled therapeutic agent in the biological sample; and
quantifying the molecular flux of the biomolecule or isotope-labeled therapeutic agent in the biological sample.

9. The method of claim 8, wherein at least two biological samples are obtained from the subject at different time points and analyzed by SILK-SIMS.

10. The method of claim 8, wherein the method or system comprises (i) electrostatically rastering a focused Cs+ primary ion beam across a defined region-of-interest (ROI) in the biological sample producing secondary ions used to measure the atomic composition of the biological sample surface; (ii) producing high-resolution quantitative image representing an in situ isotopic-histological map at 100 nm lateral resolution; (iii) acquiring one or more isotopes, optionally, in parallel; (iv) detecting and localizing a biomolecule or isotope-labeled therapeutic agent; or (v) quantitatively imaging the ratio of two stable isotopes of the same element.

11. The method of claim 8, wherein the system comprises determining the location of the biomolecule performed at a nanometer resolution, wherein the stable isotope precursor or stable isotope-labeled therapeutic agent comprises one or more of ²H, ¹³C, ¹⁵N, ¹⁸O, ¹⁷O, ³H, ¹⁴C, ³⁵S, ³²p, ¹²⁵I, ¹³¹I, ¹⁹F, ⁸¹Br ¹³C₆-leucine, ¹²C¹⁴N—, ¹³C¹⁴N—, and ¹⁶O—.

12. The method of claim 8, wherein a biomolecule is selected from the group consisting of lipids, proteins, peptides, or carbohydrates.

13. The method of claim 8, wherein the therapeutic agent is one or more of a conventional drug, a gene therapy construct, a chemotherapeutic agent, an antibiotics, a macromolecule, a protein bound drug, a cell-based therapies such as bone marrow-derived mesenchymal stem cells, an oncolytic virus, fractions of tissues or cells, a nanoparticles, a nucleic acid, a polypeptide, a siRNA, an antisense molecule, an aptamer, a ribozyme, a triple helix compound, an antibody, a small organic molecule or an inorganic molecule.

14. The method of claim 8, wherein the biological sample comprises: post-mortem tissue, tissue biopsies, cell culture, CSF, or brain tissue.

15. The method of claim 8, wherein the subject has been diagnosed with or is suspected of having Alzheimer's disease, a proteinopathy, a neurodegenerative disease, Parkinson's disease, cancer, a heart disease, or diabetes.

16. The method of claim 8, comprising measuring a ratio of ¹³C¹⁴N—/¹²C¹⁴N—, wherein if the ratio is increased compared to natural abundance, indicates an increase in pathology, optionally, Aβ plaque deposition.

17. The method of claim 16, wherein an increase ¹³C/¹²C ratio is detected with at least about 1.11%-1.25%.

18. The method of claim 13, wherein the subject has been diagnosed with cancer or is suspected of having cancer and wherein the method comprises measuring and determining the location of a nanoparticle, optionally, in parallel with the therapeutic agent.

19. The method of claim 8, wherein the subject has been diagnosed with cancer or is suspected of having cancer and wherein the method comprises measuring and determining the location of a stable isotope labeled oncolytic virus.

20. The method of claim 8, wherein the subject has been diagnosed with cancer or is suspected of having cancer and wherein the method comprises administering single dose/bolus of a stable isotope label to the subject, obtaining multiple biopsies over time.
